SEQUENCE LISTING (379 sequences)

SEQ ID NO: 1 - DNA, 120 bp, synthetic construct
  source 1..120: mol_type = other DNA
tgctgaaagctgtaccatacctgtctggtcttggctgaggtttcaatgaatggaatcccgtaactcttggccagttcgtgggcttgttttgtatcaactgtccttgttggcaaatcacac

SEQ ID NO: 2 - DNA, 120 bp, synthetic construct
  source 1..120: mol_type = other DNA
tttcaatgaatggaatcccgtaactcttggccagttcgtgggcttgttttgtatcaactgtccttgttggcaaatcacacttgtttcccactagcaccataggtacatcatccgagtctt

SEQ ID NO: 3 - DNA, 120 bp, synthetic construct
  source 1..120: mol_type = other DNA
gctattattgatggcaaatacacagaggaagccttcgcctgtcctcatgtattggtctctcatggcactgtactcttcttgtccagctgtatccagtatgtccaacaaacaggtttcacc

SEQ ID NO: 4 - DNA, 120 bp, synthetic construct
  source 1..120: mol_type = other DNA
attggtctctcatggcactgtactcttcttgtccagctgtatccagtatgtccaacaaacaggtttcaccatctataaccacttgttttctgtaagaatcctgggggtgtggagggtaag

SEQ ID NO: 5 - DNA, 120 bp, synthetic construct
  source 1..120: mol_type = other DNA
taccactgggcctcacctctatggtgggatcatattcatctacaaagtggttctggattagctggattgtcagtgcgcttttcccaacaccacctgctccaaccaccaccagtttgtact

SEQ ID NO: 6 - DNA, 120 bp, synthetic construct
  source 1..120: mol_type = other DNA
ctcacctctatggtgggatcatattcatctacaaagtggttctggattagctggattgtcagtgcgcttttcccaacaccacctgctccaaccaccaccagtttgtactcagtcatttca

SEQ ID NO: 7 - DNA, 120 bp, synthetic construct
  source 1..120: mol_type = other DNA
tttatttcagtgttacttacctgtcttgtctttgctgatgtttcaataaaaggaattccataacttcttgctaagtcctgagcctgttttgtgtctactgttctagaaggcaaatcacat

SEQ ID NO: 8 - DNA, 120 bp, synthetic construct
  source 1..120: mol_type = other DNA
tttcaataaaaggaattccataacttcttgctaagtcctgagcctgttttgtgtctactgttctagaaggcaaatcacatttatttcctactaggaccataggtacatcttcagagtcct

SEQ ID NO: 9 - DNA, 120 bp, synthetic construct
  source 1..120: mol_type = other DNA
agcctgttttgtgtctactgttctagaaggcaaatcacatttatttcctactaggaccataggtacatcttcagagtccttaactcttttaatttgttctctgggaaagaaaaaaaagtt

SEQ ID NO: 10 - DNA, 120 bp, synthetic construct
  source 1..120: mol_type = other DNA
agtattatttatggcaaatacacaaagaaagccctccccagtcctcatgtactggtccctcattgcactgtactcctcttgacctgctgtgtcgagaatatccaagagacaggtttctcc

SEQ ID NO: 11 - DNA, 120 bp, synthetic construct
  source 1..120: mol_type = other DNA
actggtccctcattgcactgtactcctcttgacctgctgtgtcgagaatatccaagagacaggtttctccatcaattactacttgcttcctgtaggaatcctgagaagggagaaacacag

SEQ ID NO: 12 - DNA, 120 bp, synthetic construct
  source 1..120: mol_type = other DNA
tttacctctattgttggatcatattcgtccacaaaatgattctgaattagctgtatcgtcaaggcactcttgcctacgccaccagctccaactaccacaagtttatattcagtcattttc

SEQ ID NO: 13 - DNA, 120 bp, synthetic construct
  source 1..120: mol_type = other DNA
gtccacaaaatgattctgaattagctgtatcgtcaaggcactcttgcctacgccaccagctccaactaccacaagtttatattcagtcattttcagcaggccttataataaaaataatga

SEQ ID NO: 14 - DNA, 120 bp, synthetic construct
  source 1..120: mol_type = other DNA
tttccaatggactattttagaagaaatggagctgtcacccacatcaagattcagaacactggtgattactatgacctgtatggaggggagaaatttgccactttggctgagttggtccag

SEQ ID NO: 15 - DNA, 120 bp, synthetic construct
  source 1..120: mol_type = other DNA
actggtgattactatgacctgtatggaggggagaaatttgccactttggctgagttggtccagtattacatggaacatcacgggcaattaaaagagaagaatggagatgtcattgagctt

SEQ ID NO: 16 - DNA, 120 bp, synthetic construct
  source 1..120: mol_type = other DNA
tcatgatgtttccttcgtaggtgttgactgcgatattgacgttcccaaaaccatccagatggtgcggtctcagaggtcagggatggtccagacagaagcacagtaccgatttatctatat

SEQ ID NO: 17 - DNA, 120 bp, synthetic construct
  source 1..120: mol_type = other DNA
gaggtcagggatggtccagacagaagcacagtaccgatttatctatatggcggtccagcattatattgaaacactacagcgcaggattgaagaagagcaggtaccagcctgagggctggc

SEQ ID NO: 18 - DNA, 120 bp, synthetic construct
  source 1..120: mol_type = other DNA
taggaaatagcagcctcacattgcccctgacaacatagttggaatcactcatgatatctcgagccaatccaaagtcacatatcttcaccactttcccgtgggtgacaagcacgttcctgg

SEQ ID NO: 19 - DNA, 120 bp, synthetic construct
  source 1..120: mol_type = other DNA
ttgcccctgacaacatagttggaatcactcatgatatctcgagccaatccaaagtcacatatcttcaccactttcccgtgggtgacaagcacgttcctggcggccaggtctctgtgaaca

SEQ ID NO: 20 - DNA, 120 bp, synthetic construct
  source 1..120: mol_type = other DNA
gttacctgacagtgtgcacgcccccagcaggttcacaatattctcgtggcttcccagctgggtcatcatcttgagttctgacatgagtgcctctctttcagagctgtctgctttttctgt

SEQ ID NO: 21 - DNA, 120 bp, synthetic construct
  source 1..120: mol_type = other DNA
cccccagcaggttcacaatattctcgtggcttcccagctgggtcatcatcttgagttctgacatgagtgcctctctttcagagctgtctgctttttctgtcaaagaaaggagcattaaaa

SEQ ID NO: 22 - DNA, 120 bp, synthetic construct
  source 1..120: mol_type = other DNA
cattccattcttaccaaactctaaattttctcttggaaactcccatttgagatcatattcatattctctgaaatcaacgtagaagtactcattatctgaggagccggtcacctgtaccat

SEQ ID NO: 23 - DNA, 120 bp, synthetic construct
  source 1..120: mol_type = other DNA
ctaaattttctcttggaaactcccatttgagatcatattcatattctctgaaatcaacgtagaagtactcattatctgaggagccggtcacctgtaccatctgtagctggctttcatacc

SEQ ID NO: 24 - DNA, 120 bp, synthetic construct
  source 1..120: mol_type = other DNA
tattacttgggagacttgtctgaacacttcttccaggtccaagatggtaatgggtatccatccgagaaacaggacgcctgacttgccgatgcttctgcgagcacttgaggtttccctata

SEQ ID NO: 25 - DNA, 120 bp, synthetic construct
  source 1..120: mol_type = other DNA
tgaacacttcttccaggtccaagatggtaatgggtatccatccgagaaacaggacgcctgacttgccgatgcttctgcgagcacttgaggtttccctatagaaaagaacgtgtgaaataa

SEQ ID NO: 26 - DNA, 120 bp, synthetic construct
  source 1..120: mol_type = other DNA
ccctctccaccctggcctacctggtcgccatgggcgtgcctgccaatggtgatgggcttggtccagccagggactaggcgtgggatgtttttgcagatgatgggctcccggaagacagtc

SEQ ID NO: 27 - DNA, 120 bp, synthetic construct
  source 1..120: mol_type = other DNA
tgccaatggtgatgggcttggtccagccagggactaggcgtgggatgtttttgcagatgatgggctcccggaagacagtcccccccaggatgttccggatagttccattgggacttttcc

SEQ ID NO: 28 - DNA, 120 bp, synthetic construct
  source 1..120: mol_type = other DNA
cactcacctggagtgagccctgctcccccctggctccttcccagcctgggcatccttgagttccaaggcctcattcagctctcggaacatctcgaagcgctcacgcccacggatctgcag

SEQ ID NO: 29 - DNA, 120 bp, synthetic construct
  source 1..120: mol_type = other DNA
tgctcccccctggctccttcccagcctgggcatccttgagttccaaggcctcattcagctctcggaacatctcgaagcgctcacgcccacggatctgcagcaacagaggagggggagaag

SEQ ID NO: 30 - DNA, 120 bp, synthetic construct
  source 1..120: mol_type = other DNA
gtgctccctgggggcagctcgtggtgaggctcccctttcttgcggagattctcttcctctgtgcgccggtctctcccaggacaggcacaaacacgcacctcaaagctgttccgtcccagt

SEQ ID NO: 31 - DNA, 120 bp, synthetic construct
  source 1..120: mol_type = other DNA
gtggtgaggctcccctttcttgcggagattctcttcctctgtgcgccggtctctcccaggacaggcacaaacacgcacctcaaagctgttccgtcccagtagattaccactactcaggat

SEQ ID NO: 32 - DNA, 120 bp, synthetic construct
  source 1..120: mol_type = other DNA
ctgacctggagtcttccagtgtgatgatggtgaggatgggcctccggttcatgccgcccatgcaggaactgttacacatgtagttgtagtggatggtggtacagtcagagccaacctagg

SEQ ID NO: 33 - DNA, 120 bp, synthetic construct
  source 1..120: mol_type = other DNA
gtgatgatggtgaggatgggcctccggttcatgccgcccatgcaggaactgttacacatgtagttgtagtggatggtggtacagtcagagccaacctaggagataacacaggcccaagat

SEQ ID NO: 34 - DNA, 120 bp, synthetic construct
  source 1..120: mol_type = other DNA
agacctcaggcggctcatagggcaccaccacactatgtcgaaaagtgtttctgtcatccaaatactccacacgcaaatttccttccactcggataagatgctgaggaggggccagaccta

SEQ ID NO: 35 - DNA, 120 bp, synthetic construct
  source 1..120: mol_type = other DNA
ggcaccaccacactatgtcgaaaagtgtttctgtcatccaaatactccacacgcaaatttccttccactcggataagatgctgaggaggggccagacctaagagcaatcagtgaggaatc

SEQ ID NO: 36 - DNA, 120 bp, synthetic construct
  source 1..120: mol_type = other DNA
ctccagccccagctgctcaccatcgctatctgagcagcgctcatggtgggggcagcgcctcacaacctccgtcatgtgctgtgactgcttgtagatggccatggcgcggacgcgggtgcc

SEQ ID NO: 37 - DNA, 120 bp, synthetic construct
  source 1..120: mol_type = other DNA
gcctcacaacctccgtcatgtgctgtgactgcttgtagatggccatggcgcggacgcgggtgccgggcgggggtgtggaatcaacccacagctgcacagggcaggtcttggccagttggc

SEQ ID NO: 38 - DNA, 120 bp, synthetic construct
  source 1..120: mol_type = other DNA
cgcggacgcgggtgccgggcgggggtgtggaatcaacccacagctgcacagggcaggtcttggccagttggcaaaacatcttgttgagggcaggggagtactgtaggaagaggaaggaga

SEQ ID NO: 39 - DNA, 120 bp, synthetic construct
  source 1..120: mol_type = other DNA
aatgcaagaagcccagacggaaaccgtagctgccctggtaggttttctgggaagggacagaagatgacaggggccaggagggggctggtgcaggggccgccggtgtaggagctgctggtg

SEQ ID NO: 40 - DNA, 120 bp, synthetic construct
  source 1..120: mol_type = other DNA
gaagggacagaagatgacaggggccaggagggggctggtgcaggggccgccggtgtaggagctgctggtgcaggggccacggggggagcagcctctggcattctgggagcttcatctgga

SEQ ID NO: 41 - DNA, 120 bp, synthetic construct
  source 1..120: mol_type = other DNA
gcccttccaatggatccactcacagtttccataggtctgaaaatgtttcctgactcagagggggctcgacgctaggatctgactgcggctcctccatggcagtgacccggaaggcagtct

SEQ ID NO: 42 - DNA, 120 bp, synthetic construct
  source 1..120: mol_type = other DNA
cacagtttccataggtctgaaaatgtttcctgactcagagggggctcgacgctaggatctgactgcggctcctccatggcagtgacccggaaggcagtctggctgctgcaagaggaaaag

SEQ ID NO: 43 - DNA, 120 bp, synthetic construct
  source 1..120: mol_type = other DNA
ttattgccaacatgacttacttgatccccataagcatgacgacctatgatgataggttttacccatccactcacaagccgggggatatttttgcagataatggcttctctgaagaccgtg

SEQ ID NO: 44 - DNA, 120 bp, synthetic construct
  source 1..120: mol_type = other DNA
aggttttacccatccactcacaagccgggggatatttttgcagataatggcttctctgaagaccgtgccacccagaatatttcgtatggtgccatttggtgatttccacatttgtttcaa

SEQ ID NO: 45 - DNA, 35 bp, synthetic construct
  source 1..35: mol_type = other DNA
caaatgctgaaagctgtaccatacctgtctggtct

SEQ ID NO: 46 - DNA, 35 bp, synthetic construct
  source 1..35: mol_type = other DNA
gctgaggtttcaatgaatggaatcccgtaactctt

SEQ ID NO: 47 - DNA, 35 bp, synthetic construct
  source 1..35: mol_type = other DNA
ccagttcgtgggcttgttttgtatcaactgtcctt

SEQ ID NO: 48 - DNA, 35 bp, synthetic construct
  source 1..35: mol_type = other DNA
tggcaaatcacacttgtttcccactagcaccatag

SEQ ID NO: 49 - DNA, 35 bp, synthetic construct
  source 1..35: mol_type = other DNA
acatcatccgagtcttttactcgcttaatctgctc

SEQ ID NO: 50 - DNA, 37 bp, synthetic construct
  source 1..37: mol_type = other DNA
acttgctattattgatggcaaatacacagaggaagcc

SEQ ID NO: 51 - DNA, 35 bp, synthetic construct
  source 1..35: mol_type = other DNA
cgcctgtcctcatgtattggtctctcatggcactg

SEQ ID NO: 52 - DNA, 35 bp, synthetic construct
  source 1..35: mol_type = other DNA
ctcttcttgtccagctgtatccagtatgtccaaca

SEQ ID NO: 53 - DNA, 40 bp, synthetic construct
  source 1..40: mol_type = other DNA
caggtttcaccatctataaccacttgttttctgtaagaat

SEQ ID NO: 54 - DNA, 35 bp, synthetic construct
  source 1..35: mol_type = other DNA
cctgggggtgtggagggtaagggggcagggaggga

SEQ ID NO: 55 - DNA, 35 bp, synthetic construct
  source 1..35: mol_type = other DNA
gggctaccactgggcctcacctctatggtgggatc

SEQ ID NO: 56 - DNA, 38 bp, synthetic construct
  source 1..38: mol_type = other DNA
attcatctacaaagtggttctggattagctggattgtc

SEQ ID NO: 57 - DNA, 35 bp, synthetic construct
  source 1..35: mol_type = other DNA
tgcgcttttcccaacaccacctgctccaaccacca

SEQ ID NO: 58 - DNA, 35 bp, synthetic construct
  source 1..35: mol_type = other DNA
agtttgtactcagtcatttcacaccagcaagaacc

SEQ ID NO: 59 - DNA, 40 bp, synthetic construct
  source 1..40: mol_type = other DNA
tatttatttcagtgttacttacctgtcttgtctttgctga

SEQ ID NO: 60 - DNA, 40 bp, synthetic construct
  source 1..40: mol_type = other DNA
tgtttcaataaaaggaattccataacttcttgctaagtcc

SEQ ID NO: 61 - DNA, 35 bp, synthetic construct
  source 1..35: mol_type = other DNA
tgagcctgttttgtgtctactgttctagaaggcaa

SEQ ID NO: 62 - DNA, 39 bp, synthetic construct
  source 1..39: mol_type = other DNA
cacatttatttcctactaggaccataggtacatcttcag

SEQ ID NO: 63 - DNA, 40 bp, synthetic construct
  source 1..40: mol_type = other DNA
gtccttaactcttttaatttgttctctgggaaagaaaaaa

SEQ ID NO: 64 - DNA, 40 bp, synthetic construct
  source 1..40: mol_type = other DNA
aagttatagcacagtcattagtaacacaaatatctttcaa

SEQ ID NO: 65 - DNA, 40 bp, synthetic construct
  source 1..40: mol_type = other DNA
tagtattatttatggcaaatacacaaagaaagccctcccc

SEQ ID NO: 66 - DNA, 35 bp, synthetic construct
  source 1..35: mol_type = other DNA
agtcctcatgtactggtccctcattgcactgtact

SEQ ID NO: 67 - DNA, 35 bp, synthetic construct
  source 1..35: mol_type = other DNA
tcttgacctgctgtgtcgagaatatccaagagaca

SEQ ID NO: 68 - DNA, 38 bp, synthetic construct
  source 1..38: mol_type = other DNA
tttctccatcaattactacttgcttcctgtaggaatcc

SEQ ID NO: 69 - DNA, 35 bp, synthetic construct
  source 1..35: mol_type = other DNA
agaagggagaaacacagtctggattattacagtgc

SEQ ID NO: 70 - DNA, 40 bp, synthetic construct
  source 1..40: mol_type = other DNA
gatttacctctattgttggatcatattcgtccacaaaatg

SEQ ID NO: 71 - DNA, 35 bp, synthetic construct
  source 1..35: mol_type = other DNA
attctgaattagctgtatcgtcaaggcactcttgc

SEQ ID NO: 72 - DNA, 35 bp, synthetic construct
  source 1..35: mol_type = other DNA
acgccaccagctccaactaccacaagtttatattc

SEQ ID NO: 73 - DNA, 40 bp, synthetic construct
  source 1..40: mol_type = other DNA
tcattttcagcaggccttataataaaaataatgaaaatgt

SEQ ID NO: 74 - DNA, 39 bp, synthetic construct
  source 1..39: mol_type = other DNA
ctttccaatggactattttagaagaaatggagctgtcac

SEQ ID NO: 75 - DNA, 37 bp, synthetic construct
  source 1..37: mol_type = other DNA
cacatcaagattcagaacactggtgattactatgacc

SEQ ID NO: 76 - DNA, 35 bp, synthetic construct
  source 1..35: mol_type = other DNA
tatggaggggagaaatttgccactttggctgagtt

SEQ ID NO: 77 - DNA, 38 bp, synthetic construct
  source 1..38: mol_type = other DNA
tccagtattacatggaacatcacgggcaattaaaagag

SEQ ID NO: 78 - DNA, 39 bp, synthetic construct
  source 1..39: mol_type = other DNA
gaatggagatgtcattgagcttaaatatcctctgaactg

SEQ ID NO: 79 - DNA, 35 bp, synthetic construct
  source 1..35: mol_type = other DNA
cttcatgatgtttccttcgtaggtgttgactgcga

SEQ ID NO: 80 - DNA, 35 bp, synthetic construct
  source 1..35: mol_type = other DNA
ttgacgttcccaaaaccatccagatggtgcggtct

SEQ ID NO: 81 - DNA, 38 bp, synthetic construct
  source 1..38: mol_type = other DNA
gtaccgatttatctatatggcggtccagcattatattg

SEQ ID NO: 82 - DNA, 35 bp, synthetic construct
  source 1..35: mol_type = other DNA
acactacagcgcaggattgaagaagagcaggtacc

SEQ ID NO: 83 - DNA, 35 bp, synthetic construct
  source 1..35: mol_type = other DNA
cctgagggctggcatgcggattctcattctcttgc

SEQ ID NO: 84 - DNA, 35 bp, synthetic construct
  source 1..35: mol_type = other DNA
taagtaggaaatagcagcctcacattgcccctgac

SEQ ID NO: 85 - DNA, 36 bp, synthetic construct
  source 1..36: mol_type = other DNA
catagttggaatcactcatgatatctcgagccaatc

SEQ ID NO: 86 - DNA, 35 bp, synthetic construct
  source 1..35: mol_type = other DNA
aagtcacatatcttcaccactttcccgtgggtgac

SEQ ID NO: 87 - DNA, 35 bp, synthetic construct
  source 1..35: mol_type = other DNA
gcacgttcctggcggccaggtctctgtgaacacac

SEQ ID NO: 88 - DNA, 35 bp, synthetic construct
  source 1..35: mol_type = other DNA
gtgggttacctgacagtgtgcacgcccccagcagg

SEQ ID NO: 89 - DNA, 35 bp, synthetic construct
  source 1..35: mol_type = other DNA
cacaatattctcgtggcttcccagctgggtcatca

SEQ ID NO: 90 - DNA, 35 bp, synthetic construct
  source 1..35: mol_type = other DNA
ttgagttctgacatgagtgcctctctttcagagct

SEQ ID NO: 91 - DNA, 40 bp, synthetic construct
  source 1..40: mol_type = other DNA
ctgctttttctgtcaaagaaaggagcattaaaaatgtaaa

SEQ ID NO: 92 - DNA, 40 bp, synthetic construct
  source 1..40: mol_type = other DNA
ggcacattccattcttaccaaactctaaattttctcttgg

SEQ ID NO: 93 - DNA, 40 bp, synthetic construct
  source 1..40: mol_type = other DNA
aaactcccatttgagatcatattcatattctctgaaatca

SEQ ID NO: 94 - DNA, 35 bp, synthetic construct
  source 1..35: mol_type = other DNA
acgtagaagtactcattatctgaggagccggtcac

SEQ ID NO: 95 - DNA, 35 bp, synthetic construct
  source 1..35: mol_type = other DNA
gtaccatctgtagctggctttcatacctaaattgc

SEQ ID NO: 96 - DNA, 36 bp, synthetic construct
  source 1..36: mol_type = other DNA
tattacttgggagacttgtctgaacacttcttccag

SEQ ID NO: 97 - DNA, 35 bp, synthetic construct
  source 1..35: mol_type = other DNA
ccaagatggtaatgggtatccatccgagaaacagg

SEQ ID NO: 98 - DNA, 35 bp, synthetic construct
  source 1..35: mol_type = other DNA
gcctgacttgccgatgcttctgcgagcacttgagg

SEQ ID NO: 99 - DNA, 37 bp, synthetic construct
  source 1..37: mol_type = other DNA
tccctatagaaaagaacgtgtgaaataagctcactgg

SEQ ID NO: 100 - DNA, 35 bp, synthetic construct
  source 1..35: mol_type = other DNA
atcccctctccaccctggcctacctggtcgccatg

SEQ ID NO: 101 - DNA, 35 bp, synthetic construct
  source 1..35: mol_type = other DNA
cgtgcctgccaatggtgatgggcttggtccagcca

SEQ ID NO: 102 - DNA, 35 bp, synthetic construct
  source 1..35: mol_type = other DNA
gactaggcgtgggatgtttttgcagatgatgggct

SEQ ID NO: 103 - DNA, 35 bp, synthetic construct
  source 1..35: mol_type = other DNA
cggaagacagtcccccccaggatgttccggatagt

SEQ ID NO: 104 - DNA, 35 bp, synthetic construct
  source 1..35: mol_type = other DNA
cattgggacttttccacatcttcttcagcttgaac

SEQ ID NO: 105 - DNA, 35 bp, synthetic construct
  source 1..35: mol_type = other DNA
aggtcactcacctggagtgagccctgctcccccct

SEQ ID NO: 106 - DNA, 35 bp, synthetic construct
  source 1..35: mol_type = other DNA
ctccttcccagcctgggcatccttgagttccaagg

SEQ ID NO: 107 - DNA, 35 bp, synthetic construct
  source 1..35: mol_type = other DNA
tcattcagctctcggaacatctcgaagcgctcacg

SEQ ID NO: 108 - DNA, 35 bp, synthetic construct
  source 1..35: mol_type = other DNA
cacggatctgcagcaacagaggagggggagaagta

SEQ ID NO: 109 - DNA, 35 bp, synthetic construct
  source 1..35: mol_type = other DNA
agtgctccctgggggcagctcgtggtgaggctccc

SEQ ID NO: 110 - DNA, 35 bp, synthetic construct
  source 1..35: mol_type = other DNA
ttcttgcggagattctcttcctctgtgcgccggtc

SEQ ID NO: 111 - DNA, 35 bp, synthetic construct
  source 1..35: mol_type = other DNA
tcccaggacaggcacaaacacgcacctcaaagctg

SEQ ID NO: 112 - DNA, 35 bp, synthetic construct
  source 1..35: mol_type = other DNA
ccgtcccagtagattaccactactcaggataggaa

SEQ ID NO: 113 - DNA, 35 bp, synthetic construct
  source 1..35: mol_type = other DNA
ctcctgacctggagtcttccagtgtgatgatggtg

SEQ ID NO: 114 - DNA, 35 bp, synthetic construct
  source 1..35: mol_type = other DNA
gatgggcctccggttcatgccgcccatgcaggaac

SEQ ID NO: 115 - DNA, 35 bp, synthetic construct
  source 1..35: mol_type = other DNA
ttacacatgtagttgtagtggatggtggtacagtc

SEQ ID NO: 116 - DNA, 35 bp, synthetic construct
  source 1..35: mol_type = other DNA
agccaacctaggagataacacaggcccaagatgag

SEQ ID NO: 117 - DNA, 35 bp, synthetic construct
  source 1..35: mol_type = other DNA
ccagacctcaggcggctcatagggcaccaccacac

SEQ ID NO: 118 - DNA, 37 bp, synthetic construct
  source 1..37: mol_type = other DNA
tgtcgaaaagtgtttctgtcatccaaatactccacac

SEQ ID NO: 119 - DNA, 35 bp, synthetic construct
  source 1..35: mol_type = other DNA
aaatttccttccactcggataagatgctgaggagg

SEQ ID NO: 120 - DNA, 35 bp, synthetic construct
  source 1..35: mol_type = other DNA
ccagacctaagagcaatcagtgaggaatcagaggc

SEQ ID NO: 121 - DNA, 35 bp, synthetic construct
  source 1..35: mol_type = other DNA
ctccagccccagctgctcaccatcgctatctgagc

SEQ ID NO: 122 - DNA, 35 bp, synthetic construct
  source 1..35: mol_type = other DNA
cgctcatggtgggggcagcgcctcacaacctccgt

SEQ ID NO: 123 - DNA, 35 bp, synthetic construct
  source 1..35: mol_type = other DNA
tgtgctgtgactgcttgtagatggccatggcgcgg

SEQ ID NO: 124 - DNA, 35 bp, synthetic construct
  source 1..35: mol_type = other DNA
gcgggtgccgggcgggggtgtggaatcaacccaca

SEQ ID NO: 125 - DNA, 35 bp, synthetic construct
  source 1..35: mol_type = other DNA
tgcacagggcaggtcttggccagttggcaaaacat

SEQ ID NO: 126 - DNA, 35 bp, synthetic construct
  source 1..35: mol_type = other DNA
tgttgagggcaggggagtactgtaggaagaggaag

SEQ ID NO: 127 - DNA, 35 bp, synthetic construct
  source 1..35: mol_type = other DNA
gacagagttgaaagtcagggcacaagtgaacagat

SEQ ID NO: 128 - DNA, 35 bp, synthetic construct
  source 1..35: mol_type = other DNA
aatgcaagaagcccagacggaaaccgtagctgccc

SEQ ID NO: 129 - DNA, 35 bp, synthetic construct
  source 1..35: mol_type = other DNA
gtaggttttctgggaagggacagaagatgacaggg

SEQ ID NO: 130 - DNA, 35 bp, synthetic construct
  source 1..35: mol_type = other DNA
caggagggggctggtgcaggggccgccggtgtagg

SEQ ID NO: 131 - DNA, 35 bp, synthetic construct
  source 1..35: mol_type = other DNA
ctgctggtgcaggggccacggggggagcagcctct

SEQ ID NO: 132 - DNA, 35 bp, synthetic construct
  source 1..35: mol_type = other DNA
cattctgggagcttcatctggacctgggtcttcag

SEQ ID NO: 133 - DNA, 35 bp, synthetic construct
  source 1..35: mol_type = other DNA
gcccttccaatggatccactcacagtttccatagg

SEQ ID NO: 134 - DNA, 35 bp, synthetic construct
  source 1..35: mol_type = other DNA
tgaaaatgtttcctgactcagagggggctcgacgc

SEQ ID NO: 135 - DNA, 35 bp, synthetic construct
  source 1..35: mol_type = other DNA
ggatctgactgcggctcctccatggcagtgacccg

SEQ ID NO: 136 - DNA, 35 bp, synthetic construct
  source 1..35: mol_type = other DNA
aggcagtctggctgctgcaagaggaaaagtgggga

SEQ ID NO: 137 - DNA, 37 bp, synthetic construct
  source 1..37: mol_type = other DNA
cattattgccaacatgacttacttgatccccataagc

SEQ ID NO: 138 - DNA, 35 bp, synthetic construct
  source 1..35: mol_type = other DNA
gacgacctatgatgataggttttacccatccactc

SEQ ID NO: 139 - DNA, 35 bp, synthetic construct
  source 1..35: mol_type = other DNA
aagccgggggatatttttgcagataatggcttctc

SEQ ID NO: 140 - DNA, 35 bp, synthetic construct
  source 1..35: mol_type = other DNA
aagaccgtgccacccagaatatttcgtatggtgcc

SEQ ID NO: 141 - DNA, 40 bp, synthetic construct
  source 1..40: mol_type = other DNA
ttggtgatttccacatttgtttcaacttgaactcctcaac

SEQ ID NO: 142 - DNA, 51 bp, synthetic construct
  source 1..51: mol_type = other DNA
cgtcggtccaaatgctgaaagctgtaccatacctgtctggtctgaccgacg

SEQ ID NO: 143 - DNA, 51 bp, synthetic construct
  source 1..51: mol_type = other DNA
cgtcggtcgctgaggtttcaatgaatggaatcccgtaactcttgaccgacg

SEQ ID NO: 144 - DNA, 51 bp, synthetic construct
  source 1..51: mol_type = other DNA
cgtcggtcccagttcgtgggcttgttttgtatcaactgtccttgaccgacg

SEQ ID NO: 145 - DNA, 51 bp, synthetic construct
  source 1..51: mol_type = other DNA
cgtcggtctggcaaatcacacttgtttcccactagcaccataggaccgacg

SEQ ID NO: 146 - DNA, 51 bp, synthetic construct
  source 1..51: mol_type = other DNA
cgtcggtcacatcatccgagtcttttactcgcttaatctgctcgaccgacg

SEQ ID NO: 147 - DNA, 53 bp, synthetic construct
  source 1..53: mol_type = other DNA
cgtcggtcacttgctattattgatggcaaatacacagaggaagccgaccgacg

SEQ ID NO: 148 - DNA, 51 bp, synthetic construct
  source 1..51: mol_type = other DNA
cgtcggtccgcctgtcctcatgtattggtctctcatggcactggaccgacg

SEQ ID NO: 149 - DNA, 51 bp, synthetic construct
  source 1..51: mol_type = other DNA
cgtcggtcctcttcttgtccagctgtatccagtatgtccaacagaccgacg

SEQ ID NO: 150 - DNA, 56 bp, synthetic construct
  source 1..56: mol_type = other DNA
cgtcggtccaggtttcaccatctataaccacttgttttctgtaagaatgaccgacg

SEQ ID NO: 151 - DNA, 51 bp, synthetic construct
  source 1..51: mol_type = other DNA
cgtcggtccctgggggtgtggagggtaagggggcagggagggagaccgacg

SEQ ID NO: 152 - DNA, 51 bp, synthetic construct
  source 1..51: mol_type = other DNA
cgtcggtcgggctaccactgggcctcacctctatggtgggatcgaccgacg

SEQ ID NO: 153 - DNA, 54 bp, synthetic construct
  source 1..54: mol_type = other DNA
cgtcggtcattcatctacaaagtggttctggattagctggattgtcgaccgacg

SEQ ID NO: 154 - DNA, 51 bp, synthetic construct
  source 1..51: mol_type = other DNA
cgtcggtctgcgcttttcccaacaccacctgctccaaccaccagaccgacg

SEQ ID NO: 155 - DNA, 51 bp, synthetic construct
  source 1..51: mol_type = other DNA
cgtcggtcagtttgtactcagtcatttcacaccagcaagaaccgaccgacg

SEQ ID NO: 156 - DNA, 56 bp, synthetic construct
  source 1..56: mol_type = other DNA
cgtcggtctatttatttcagtgttacttacctgtcttgtctttgctgagaccgacg

SEQ ID NO: 157 - DNA, 56 bp, synthetic construct
  source 1..56: mol_type = other DNA
cgtcggtctgtttcaataaaaggaattccataacttcttgctaagtccgaccgacg

SEQ ID NO: 158 - DNA, 51 bp, synthetic construct
  source 1..51: mol_type = other DNA
cgtcggtctgagcctgttttgtgtctactgttctagaaggcaagaccgacg

SEQ ID NO: 159 - DNA, 55 bp, synthetic construct
  source 1..55: mol_type = other DNA
cgtcggtccacatttatttcctactaggaccataggtacatcttcaggaccgacg

SEQ ID NO: 160 - DNA, 56 bp, synthetic construct
  source 1..56: mol_type = other DNA
cgtcggtcgtccttaactcttttaatttgttctctgggaaagaaaaaagaccgacg

SEQ ID NO: 161 - DNA, 56 bp, synthetic construct
  source 1..56: mol_type = other DNA
cgtcggtcaagttatagcacagtcattagtaacacaaatatctttcaagaccgacg

SEQ ID NO: 162 - DNA, 56 bp, synthetic construct
  source 1..56: mol_type = other DNA
cgtcggtctagtattatttatggcaaatacacaaagaaagccctccccgaccgacg

SEQ ID NO: 163 - DNA, 51 bp, synthetic construct
  source 1..51: mol_type = other DNA
cgtcggtcagtcctcatgtactggtccctcattgcactgtactgaccgacg

SEQ ID NO: 164 - DNA, 51 bp, synthetic construct
  source 1..51: mol_type = other DNA
cgtcggtctcttgacctgctgtgtcgagaatatccaagagacagaccgacg

SEQ ID NO: 165 - DNA, 54 bp, synthetic construct
  source 1..54: mol_type = other DNA
cgtcggtctttctccatcaattactacttgcttcctgtaggaatccgaccgacg

SEQ ID NO: 166 - DNA, 51 bp, synthetic construct
  source 1..51: mol_type = other DNA
cgtcggtcagaagggagaaacacagtctggattattacagtgcgaccgacg

SEQ ID NO: 167 - DNA, 56 bp, synthetic construct
  source 1..56: mol_type = other DNA
cgtcggtcgatttacctctattgttggatcatattcgtccacaaaatggaccgacg

SEQ ID NO: 168 - DNA, 51 bp, synthetic construct
  source 1..51: mol_type = other DNA
cgtcggtcattctgaattagctgtatcgtcaaggcactcttgcgaccgacg

SEQ ID NO: 169 - DNA, 51 bp, synthetic construct
  source 1..51: mol_type = other DNA
cgtcggtcacgccaccagctccaactaccacaagtttatattcgaccgacg

SEQ ID NO: 170 - DNA, 56 bp, synthetic construct
  source 1..56: mol_type = other DNA
cgtcggtctcattttcagcaggccttataataaaaataatgaaaatgtgaccgacg

SEQ ID NO: 171 - DNA, 55 bp, synthetic construct
  source 1..55: mol_type = other DNA
cgtcggtcctttccaatggactattttagaagaaatggagctgtcacgaccgacg

SEQ ID NO: 172 - DNA, 53 bp, synthetic construct
  source 1..53: mol_type = other DNA
cgtcggtccacatcaagattcagaacactggtgattactatgaccgaccgacg

SEQ ID NO: 173 - DNA, 51 bp, synthetic construct
  source 1..51: mol_type = other DNA
cgtcggtctatggaggggagaaatttgccactttggctgagttgaccgacg

SEQ ID NO: 174 - DNA, 54 bp, synthetic construct
  source 1..54: mol_type = other DNA
cgtcggtctccagtattacatggaacatcacgggcaattaaaagaggaccgacg

SEQ ID NO: 175 - DNA, 55 bp, synthetic construct
  source 1..55: mol_type = other DNA
cgtcggtcgaatggagatgtcattgagcttaaatatcctctgaactggaccgacg

SEQ ID NO: 176 - DNA, 51 bp, synthetic construct
  source 1..51: mol_type = other DNA
cgtcggtccttcatgatgtttccttcgtaggtgttgactgcgagaccgacg

SEQ ID NO: 177 - DNA, 51 bp, synthetic construct
  source 1..51: mol_type = other DNA
cgtcggtcttgacgttcccaaaaccatccagatggtgcggtctgaccgacg

SEQ ID NO: 178 - DNA, 54 bp, synthetic construct
  source 1..54: mol_type = other DNA
cgtcggtcgtaccgatttatctatatggcggtccagcattatattggaccgacg

SEQ ID NO: 179 - DNA, 51 bp, synthetic construct
  source 1..51: mol_type = other DNA
cgtcggtcacactacagcgcaggattgaagaagagcaggtaccgaccgacg

SEQ ID NO: 180 - DNA, 51 bp, synthetic construct
  source 1..51: mol_type = other DNA
cgtcggtccctgagggctggcatgcggattctcattctcttgcgaccgacg

SEQ ID NO: 181 - DNA, 51 bp, synthetic construct
  source 1..51: mol_type = other DNA
cgtcggtctaagtaggaaatagcagcctcacattgcccctgacgaccgacg

SEQ ID NO: 182 - DNA, 52 bp, synthetic construct
  source 1..52: mol_type = other DNA
cgtcggtccatagttggaatcactcatgatatctcgagccaatcgaccgacg

SEQ ID NO: 183 - DNA, 51 bp, synthetic construct
  source 1..51: mol_type = other DNA
cgtcggtcaagtcacatatcttcaccactttcccgtgggtgacgaccgacg

SEQ ID NO: 184 - DNA, 51 bp, synthetic construct
  source 1..51: mol_type = other DNA
cgtcggtcgcacgttcctggcggccaggtctctgtgaacacacgaccgacg

SEQ ID NO: 185 - DNA, 51 bp, synthetic construct
  source 1..51: mol_type = other DNA
cgtcggtcgtgggttacctgacagtgtgcacgcccccagcagggaccgacg

SEQ ID NO: 186 - DNA, 51 bp, synthetic construct
  source 1..51: mol_type = other DNA
cgtcggtccacaatattctcgtggcttcccagctgggtcatcagaccgacg

SEQ ID NO: 187 - DNA, 51 bp, synthetic construct
  source 1..51: mol_type = other DNA
cgtcggtcttgagttctgacatgagtgcctctctttcagagctgaccgacg

SEQ ID NO: 188 - DNA, 56 bp, synthetic construct
  source 1..56: mol_type = other DNA
cgtcggtcctgctttttctgtcaaagaaaggagcattaaaaatgtaaagaccgacg

SEQ ID NO: 189 - DNA, 56 bp, synthetic construct
  source 1..56: mol_type = other DNA
cgtcggtcggcacattccattcttaccaaactctaaattttctcttgggaccgacg

SEQ ID NO: 190 - DNA, 56 bp, synthetic construct
  source 1..56: mol_type = other DNA
cgtcggtcaaactcccatttgagatcatattcatattctctgaaatcagaccgacg

SEQ ID NO: 191 - DNA, 51 bp, synthetic construct
  source 1..51: mol_type = other DNA
cgtcggtcacgtagaagtactcattatctgaggagccggtcacgaccgacg

SEQ ID NO: 192 - DNA, 51 bp, synthetic construct
  source 1..51: mol_type = other DNA
cgtcggtcgtaccatctgtagctggctttcatacctaaattgcgaccgacg

SEQ ID NO: 193 - DNA, 52 bp, synthetic construct
  source 1..52: mol_type = other DNA
cgtcggtctattacttgggagacttgtctgaacacttcttccaggaccgacg

SEQ ID NO: 194 - DNA, 51 bp, synthetic construct
  source 1..51: mol_type = other DNA
cgtcggtcccaagatggtaatgggtatccatccgagaaacagggaccgacg

SEQ ID NO: 195 - DNA, 51 bp, synthetic construct
  source 1..51: mol_type = other DNA
cgtcggtcgcctgacttgccgatgcttctgcgagcacttgagggaccgacg

SEQ ID NO: 196 - DNA, 53 bp, synthetic construct
  source 1..53: mol_type = other DNA
cgtcggtctccctatagaaaagaacgtgtgaaataagctcactgggaccgacg

SEQ ID NO: 197 - DNA, 51 bp, synthetic construct
  source 1..51: mol_type = other DNA
cgtcggtcatcccctctccaccctggcctacctggtcgccatggaccgacg

SEQ ID NO: 198 - DNA, 51 bp, synthetic construct
  source 1..51: mol_type = other DNA
cgtcggtccgtgcctgccaatggtgatgggcttggtccagccagaccgacg

SEQ ID NO: 199 - DNA, 51 bp, synthetic construct
  source 1..51: mol_type = other DNA
cgtcggtcgactaggcgtgggatgtttttgcagatgatgggctgaccgacg

SEQ ID NO: 200 - DNA, 51 bp, synthetic construct
  source 1..51: mol_type = other DNA
cgtcggtccggaagacagtcccccccaggatgttccggatagtgaccgacg

SEQ ID NO: 201 - DNA, 51 bp, synthetic construct
  source 1..51: mol_type = other DNA
cgtcggtccattgggacttttccacatcttcttcagcttgaacgaccgacg

SEQ ID NO: 202 - DNA, 51 bp, synthetic construct
  source 1..51: mol_type = other DNA
cgtcggtcaggtcactcacctggagtgagccctgctcccccctgaccgacg

SEQ ID NO: 203 - DNA, 51 bp, synthetic construct
  source 1..51: mol_type = other DNA
cgtcggtcctccttcccagcctgggcatccttgagttccaagggaccgacg

SEQ ID NO: 204 - DNA, 51 bp, synthetic construct
  source 1..51: mol_type = other DNA
cgtcggtctcattcagctctcggaacatctcgaagcgctcacggaccgacg

SEQ ID NO: 205 - DNA, 51 bp, synthetic construct
  source 1..51: mol_type = other DNA
cgtcggtccacggatctgcagcaacagaggagggggagaagtagaccgacg

SEQ ID NO: 206 - DNA, 51 bp, synthetic construct
  source 1..51: mol_type = other DNA
cgtcggtcagtgctccctgggggcagctcgtggtgaggctcccgaccgacg

SEQ ID NO: 207 - DNA, 51 bp, synthetic construct
  source 1..51: mol_type = other DNA
cgtcggtcttcttgcggagattctcttcctctgtgcgccggtcgaccgacg

SEQ ID NO: 208 - DNA, 51 bp, synthetic construct
  source 1..51: mol_type = other DNA
cgtcggtctcccaggacaggcacaaacacgcacctcaaagctggaccgacg

SEQ ID NO: 209 - DNA, 51 bp, synthetic construct
  source 1..51: mol_type = other DNA
cgtcggtcccgtcccagtagattaccactactcaggataggaagaccgacg

SEQ ID NO: 210 - DNA, 51 bp, synthetic construct
  source 1..51: mol_type = other DNA
cgtcggtcctcctgacctggagtcttccagtgtgatgatggtggaccgacg

SEQ ID NO: 211 - DNA, 51 bp, synthetic construct
  source 1..51: mol_type = other DNA
cgtcggtcgatgggcctccggttcatgccgcccatgcaggaacgaccgacg

SEQ ID NO: 212 - DNA, 51 bp, synthetic construct
  source 1..51: mol_type = other DNA
cgtcggtcttacacatgtagttgtagtggatggtggtacagtcgaccgacg

SEQ ID NO: 213 - DNA, 51 bp, synthetic construct
  source 1..51: mol_type = other DNA
cgtcggtcagccaacctaggagataacacaggcccaagatgaggaccgacg

SEQ ID NO: 214 - DNA, 51 bp, synthetic construct
  source 1..51: mol_type = other DNA
cgtcggtcccagacctcaggcggctcatagggcaccaccacacgaccgacg

SEQ ID NO: 215 - DNA, 53 bp, synthetic construct
  source 1..53: mol_type = other DNA
cgtcggtctgtcgaaaagtgtttctgtcatccaaatactccacacgaccgacg

SEQ ID NO: 216 - DNA, 51 bp, synthetic construct
  source 1..51: mol_type = other DNA
cgtcggtcaaatttccttccactcggataagatgctgaggagggaccgacg

SEQ ID NO: 217 - DNA, 51 bp, synthetic construct
  source 1..51: mol_type = other DNA
cgtcggtcccagacctaagagcaatcagtgaggaatcagaggcgaccgacg

SEQ ID NO: 218 - DNA, 51 bp, synthetic construct
  source 1..51: mol_type = other DNA
cgtcggtcctccagccccagctgctcaccatcgctatctgagcgaccgacg

SEQ ID NO: 219 - DNA, 51 bp, synthetic construct
  source 1..51: mol_type = other DNA
cgtcggtccgctcatggtgggggcagcgcctcacaacctccgtgaccgacg

SEQ ID NO: 220 - DNA, 51 bp, synthetic construct
  source 1..51: mol_type = other DNA
cgtcggtctgtgctgtgactgcttgtagatggccatggcgcgggaccgacg

SEQ ID NO: 221 - DNA, 51 bp, synthetic construct
  source 1..51: mol_type = other DNA
cgtcggtcgcgggtgccgggcgggggtgtggaatcaacccacagaccgacg

SEQ ID NO: 222 - DNA, 51 bp, synthetic construct
  source 1..51: mol_type = other DNA
cgtcggtctgcacagggcaggtcttggccagttggcaaaacatgaccgacg

SEQ ID NO: 223 - DNA, 51 bp, synthetic construct
  source 1..51: mol_type = other DNA
cgtcggtctgttgagggcaggggagtactgtaggaagaggaaggaccgacg

SEQ ID NO: 224 - DNA, 51 bp, synthetic construct
  source 1..51: mol_type = other DNA
cgtcggtcgacagagttgaaagtcagggcacaagtgaacagatgaccgacg

SEQ ID NO: 225 - DNA, 51 bp, synthetic construct
  source 1..51: mol_type = other DNA
cgtcggtcaatgcaagaagcccagacggaaaccgtagctgcccgaccgacg

SEQ ID NO: 226 - DNA, 51 bp, synthetic construct
  source 1..51: mol_type = other DNA
cgtcggtcgtaggttttctgggaagggacagaagatgacaggggaccgacg

SEQ ID NO: 227 - DNA, 51 bp, synthetic construct
  source 1..51: mol_type = other DNA
cgtcggtccaggagggggctggtgcaggggccgccggtgtagggaccgacg

SEQ ID NO: 228 - DNA, 51 bp, synthetic construct
  source 1..51: mol_type = other DNA
cgtcggtcctgctggtgcaggggccacggggggagcagcctctgaccgacg

SEQ ID NO: 229 - DNA, 51 bp, synthetic construct
  source 1..51: mol_type = other DNA
cgtcggtccattctgggagcttcatctggacctgggtcttcaggaccgacg

SEQ ID NO: 230 - DNA, 51 bp, synthetic construct
  source 1..51: mol_type = other DNA
cgtcggtcgcccttccaatggatccactcacagtttccatagggaccgacg

SEQ ID NO: 231 - DNA, 51 bp, synthetic construct
  source 1..51: mol_type = other DNA
cgtcggtctgaaaatgtttcctgactcagagggggctcgacgcgaccgacg

SEQ ID NO: 232 - DNA, 51 bp, synthetic construct
  source 1..51: mol_type = other DNA
cgtcggtcggatctgactgcggctcctccatggcagtgacccggaccgacg

SEQ ID NO: 233 - DNA, 51 bp, synthetic construct
  source 1..51: mol_type = other DNA
cgtcggtcaggcagtctggctgctgcaagaggaaaagtggggagaccgacg

SEQ ID NO: 234 - DNA, 53 bp, synthetic construct
  source 1..53: mol_type = other DNA
cgtcggtccattattgccaacatgacttacttgatccccataagcgaccgacg

SEQ ID NO: 235 - DNA, 51 bp, synthetic construct
  source 1..51: mol_type = other DNA
cgtcggtcgacgacctatgatgataggttttacccatccactcgaccgacg

SEQ ID NO: 236 - DNA, 51 bp, synthetic construct
  source 1..51: mol_type = other DNA
cgtcggtcaagccgggggatatttttgcagataatggcttctcgaccgacg

SEQ ID NO: 237 - DNA, 51 bp, synthetic construct
  source 1..51: mol_type = other DNA
cgtcggtcaagaccgtgccacccagaatatttcgtatggtgccgaccgacg

SEQ ID NO: 238 - DNA, 56 bp, synthetic construct
  source 1..56: mol_type = other DNA
cgtcggtcttggtgatttccacatttgtttcaacttgaactcctcaacgaccgacg

SEQ ID NO: 239 - DNA, 120 bp, synthetic construct
  source 1..120: mol_type = other DNA
gtgtgatttgccaacaaggacagttgatacaaaacaagcccacgaactggccaagagttacgggattccattcattgaaacctcagccaagaccagacaggtatggtacagctttcagca

SEQ ID NO: 240 - DNA, 120 bp, synthetic construct
  source 1..120: mol_type = other DNA
aagactcggatgatgtacctatggtgctagtgggaaacaagtgtgatttgccaacaaggacagttgatacaaaacaagcccacgaactggccaagagttacgggattccattcattgaaa

SEQ ID NO: 241 - DNA, 120 bp, synthetic construct
  source 1..120: mol_type = other DNA
ggtgaaacctgtttgttggacatactggatacagctggacaagaagagtacagtgccatgagagaccaatacatgaggacaggcgaaggcttcctctgtgtatttgccatcaataatagc

SEQ ID NO: 242 - DNA, 120 bp, synthetic construct
  source 1..120: mol_type = other DNA
cttaccctccacacccccaggattcttacagaaaacaagtggttatagatggtgaaacctgtttgttggacatactggatacagctggacaagaagagtacagtgccatgagagaccaat

SEQ ID NO: 243 - DNA, 120 bp, synthetic construct
  source 1..120: mol_type = other DNA
agtacaaactggtggtggttggagcaggtggtgttgggaaaagcgcactgacaatccagctaatccagaaccactttgtagatgaatatgatcccaccatagaggtgaggcccagtggta

SEQ ID NO: 244 - DNA, 120 bp, synthetic construct
  source 1..120: mol_type = other DNA
tgaaatgactgagtacaaactggtggtggttggagcaggtggtgttgggaaaagcgcactgacaatccagctaatccagaaccactttgtagatgaatatgatcccaccatagaggtgag

SEQ ID NO: 245 - DNA, 120 bp, synthetic construct
  source 1..120: mol_type = other DNA
atgtgatttgccttctagaacagtagacacaaaacaggctcaggacttagcaagaagttatggaattccttttattgaaacatcagcaaagacaagacaggtaagtaacactgaaataaa

SEQ ID NO: 246 - DNA, 120 bp, synthetic construct
  source 1..120: mol_type = other DNA
aggactctgaagatgtacctatggtcctagtaggaaataaatgtgatttgccttctagaacagtagacacaaaacaggctcaggacttagcaagaagttatggaattccttttattgaaa

SEQ ID NO: 247 - DNA, 120 bp, synthetic construct
  source 1..120: mol_type = other DNA
aacttttttttctttcccagagaacaaattaaaagagttaaggactctgaagatgtacctatggtcctagtaggaaataaatgtgatttgccttctagaacagtagacacaaaacaggct

SEQ ID NO: 248 - DNA, 120 bp, synthetic construct
  source 1..120: mol_type = other DNA
ggagaaacctgtctcttggatattctcgacacagcaggtcaagaggagtacagtgcaatgagggaccagtacatgaggactggggagggctttctttgtgtatttgccataaataatact

SEQ ID NO: 249 - DNA, 120 bp, synthetic construct
  source 1..120: mol_type = other DNA
ctgtgtttctcccttctcaggattcctacaggaagcaagtagtaattgatggagaaacctgtctcttggatattctcgacacagcaggtcaagaggagtacagtgcaatgagggaccagt

SEQ ID NO: 250 - DNA, 120 bp, synthetic construct
  source 1..120: mol_type = other DNA
gaaaatgactgaatataaacttgtggtagttggagctggtggcgtaggcaagagtgccttgacgatacagctaattcagaatcattttgtggacgaatatgatccaacaatagaggtaaa

SEQ ID NO: 251 - DNA, 120 bp, synthetic construct
  source 1..120: mol_type = other DNA
tcattatttttattataaggcctgctgaaaatgactgaatataaacttgtggtagttggagctggtggcgtaggcaagagtgccttgacgatacagctaattcagaatcattttgtggac

SEQ ID NO: 252 - DNA, 120 bp, synthetic construct
  source 1..120: mol_type = other DNA
ctggaccaactcagccaaagtggcaaatttctcccctccatacaggtcatagtaatcaccagtgttctgaatcttgatgtgggtgacagctccatttcttctaaaatagtccattggaaa

SEQ ID NO: 253 - DNA, 120 bp, synthetic construct
  source 1..120: mol_type = other DNA
aagctcaatgacatctccattcttctcttttaattgcccgtgatgttccatgtaatactggaccaactcagccaaagtggcaaatttctcccctccatacaggtcatagtaatcaccagt

SEQ ID NO: 254 - DNA, 120 bp, synthetic construct
  source 1..120: mol_type = other DNA
atatagataaatcggtactgtgcttctgtctggaccatccctgacctctgagaccgcaccatctggatggttttgggaacgtcaatatcgcagtcaacacctacgaaggaaacatcatga

SEQ ID NO: 255 - DNA, 120 bp, synthetic construct
  source 1..120: mol_type = other DNA
gccagccctcaggctggtacctgctcttcttcaatcctgcgctgtagtgtttcaatataatgctggaccgccatatagataaatcggtactgtgcttctgtctggaccatccctgacctc

SEQ ID NO: 256 - DNA, 120 bp, synthetic construct
  source 1..120: mol_type = other DNA
ccaggaacgtgcttgtcacccacgggaaagtggtgaagatatgtgactttggattggctcgagatatcatgagtgattccaactatgttgtcaggggcaatgtgaggctgctatttccta

SEQ ID NO: 257 - DNA, 120 bp, synthetic construct
  source 1..120: mol_type = other DNA
tgttcacagagacctggccgccaggaacgtgcttgtcacccacgggaaagtggtgaagatatgtgactttggattggctcgagatatcatgagtgattccaactatgttgtcaggggcaa

SEQ ID NO: 258 - DNA, 120 bp, synthetic construct
  source 1..120: mol_type = other DNA
acagaaaaagcagacagctctgaaagagaggcactcatgtcagaactcaagatgatgacccagctgggaagccacgagaatattgtgaacctgctgggggcgtgcacactgtcaggtaac

SEQ ID NO: 259 - DNA, 120 bp, synthetic construct
  source 1..120: mol_type = other DNA
ttttaatgctcctttctttgacagaaaaagcagacagctctgaaagagaggcactcatgtcagaactcaagatgatgacccagctgggaagccacgagaatattgtgaacctgctggggg

SEQ ID NO: 260 - DNA, 120 bp, synthetic construct
  source 1..120: mol_type = other DNA
atggtacaggtgaccggctcctcagataatgagtacttctacgttgatttcagagaatatgaatatgatctcaaatgggagtttccaagagaaaatttagagtttggtaagaatggaatg

SEQ ID NO: 261 - DNA, 120 bp, synthetic construct
  source 1..120: mol_type = other DNA
ggtatgaaagccagctacagatggtacaggtgaccggctcctcagataatgagtacttctacgttgatttcagagaatatgaatatgatctcaaatgggagtttccaagagaaaatttag

SEQ ID NO: 262 - DNA, 120 bp, synthetic construct
  source 1..120: mol_type = other DNA
tatagggaaacctcaagtgctcgcagaagcatcggcaagtcaggcgtcctgtttctcggatggatacccattaccatcttggacctggaagaagtgttcagacaagtctcccaagtaata

SEQ ID NO: 263 - DNA, 120 bp, synthetic construct
  source 1..120: mol_type = other DNA
ttatttcacacgttcttttctatagggaaacctcaagtgctcgcagaagcatcggcaagtcaggcgtcctgtttctcggatggatacccattaccatcttggacctggaagaagtgttca

SEQ ID NO: 264 - DNA, 120 bp, synthetic construct
  source 1..120: mol_type = other DNA
gactgtcttccgggagcccatcatctgcaaaaacatcccacgcctagtccctggctggaccaagcccatcaccattggcaggcacgcccatggcgaccaggtaggccagggtggagaggg

SEQ ID NO: 265 - DNA, 120 bp, synthetic construct
  source 1..120: mol_type = other DNA
ggaaaagtcccaatggaactatccggaacatcctgggggggactgtcttccgggagcccatcatctgcaaaaacatcccacgcctagtccctggctggaccaagcccatcaccattggca

SEQ ID NO: 266 - DNA, 120 bp, synthetic construct
  source 1..120: mol_type = other DNA
ctgcagatccgtgggcgtgagcgcttcgagatgttccgagagctgaatgaggccttggaactcaaggatgcccaggctgggaaggagccaggggggagcagggctcactccaggtgagtg

SEQ ID NO: 267 - DNA, 120 bp, synthetic construct
  source 1..120: mol_type = other DNA
cttctccccctcctctgttgctgcagatccgtgggcgtgagcgcttcgagatgttccgagagctgaatgaggccttggaactcaaggatgcccaggctgggaaggagccaggggggagca

SEQ ID NO: 268 - DNA, 120 bp, synthetic construct
  source 1..120: mol_type = other DNA
actgggacggaacagctttgaggtgcgtgtttgtgcctgtcctgggagagaccggcgcacagaggaagagaatctccgcaagaaaggggagcctcaccacgagctgcccccagggagcac

SEQ ID NO: 269 - DNA, 120 bp, synthetic construct
  source 1..120: mol_type = other DNA
atcctgagtagtggtaatctactgggacggaacagctttgaggtgcgtgtttgtgcctgtcctgggagagaccggcgcacagaggaagagaatctccgcaagaaaggggagcctcaccac

SEQ ID NO: 270 - DNA, 120 bp, synthetic construct
  source 1..120: mol_type = other DNA
cctaggttggctctgactgtaccaccatccactacaactacatgtgtaacagttcctgcatgggcggcatgaaccggaggcccatcctcaccatcatcacactggaagactccaggtcag

SEQ ID NO: 271 - DNA, 120 bp, synthetic construct
  source 1..120: mol_type = other DNA
atcttgggcctgtgttatctcctaggttggctctgactgtaccaccatccactacaactacatgtgtaacagttcctgcatgggcggcatgaaccggaggcccatcctcaccatcatcac

SEQ ID NO: 272 - DNA, 120 bp, synthetic construct
  source 1..120: mol_type = other DNA
taggtctggcccctcctcagcatcttatccgagtggaaggaaatttgcgtgtggagtatttggatgacagaaacacttttcgacatagtgtggtggtgccctatgagccgcctgaggtct

SEQ ID NO: 273 - DNA, 120 bp, synthetic construct
  source 1..120: mol_type = other DNA
gattcctcactgattgctcttaggtctggcccctcctcagcatcttatccgagtggaaggaaatttgcgtgtggagtatttggatgacagaaacacttttcgacatagtgtggtggtgcc

SEQ ID NO: 274 - DNA, 120 bp, synthetic construct
  source 1..120: mol_type = other DNA
ggcacccgcgtccgcgccatggccatctacaagcagtcacagcacatgacggaggttgtgaggcgctgcccccaccatgagcgctgctcagatagcgatggtgagcagctggggctggag

SEQ ID NO: 275 - DNA, 120 bp, synthetic construct
  source 1..120: mol_type = other DNA
gccaactggccaagacctgccctgtgcagctgtgggttgattccacacccccgcccggcacccgcgtccgcgccatggccatctacaagcagtcacagcacatgacggaggttgtgaggc

SEQ ID NO: 276 - DNA, 120 bp, synthetic construct
  source 1..120: mol_type = other DNA
tctccttcctcttcctacagtactcccctgccctcaacaagatgttttgccaactggccaagacctgccctgtgcagctgtgggttgattccacacccccgcccggcacccgcgtccgcg

SEQ ID NO: 277 - DNA, 120 bp, synthetic construct
  source 1..120: mol_type = other DNA
caccagcagctcctacaccggcggcccctgcaccagccccctcctggcccctgtcatcttctgtcccttcccagaaaacctaccagggcagctacggtttccgtctgggcttcttgcatt

SEQ ID NO: 278 - DNA, 120 bp, synthetic construct
  source 1..120: mol_type = other DNA
tccagatgaagctcccagaatgccagaggctgctccccccgtggcccctgcaccagcagctcctacaccggcggcccctgcaccagccccctcctggcccctgtcatcttctgtcccttc

SEQ ID NO: 279 - DNA, 120 bp, synthetic construct
  source 1..120: mol_type = other DNA
agactgccttccgggtcactgccatggaggagccgcagtcagatcctagcgtcgagccccctctgagtcaggaaacattttcagacctatggaaactgtgagtggatccattggaagggc

SEQ ID NO: 280 - DNA, 120 bp, synthetic construct
  source 1..120: mol_type = other DNA
cttttcctcttgcagcagccagactgccttccgggtcactgccatggaggagccgcagtcagatcctagcgtcgagccccctctgagtcaggaaacattttcagacctatggaaactgtg

SEQ ID NO: 281 - DNA, 120 bp, synthetic construct
  source 1..120: mol_type = other DNA
cacggtcttcagagaagccattatctgcaaaaatatcccccggcttgtgagtggatgggtaaaacctatcatcataggtcgtcatgcttatggggatcaagtaagtcatgttggcaataa

SEQ ID NO: 282 - DNA, 120 bp, synthetic construct
  source 1..120: mol_type = other DNA
ttgaaacaaatgtggaaatcaccaaatggcaccatacgaaatattctgggtggcacggtcttcagagaagccattatctgcaaaaatatcccccggcttgtgagtggatgggtaaaacct

SEQ ID NO: 283 - DNA, 51 bp, synthetic construct
  source 1..51: mol_type = other DNA
cgtcggtcagaccagacaggtatggtacagctttcagcatttggaccgacg

SEQ ID NO: 284 - DNA, 51 bp, synthetic construct
  source 1..51: mol_type = other DNA
cgtcggtcaagagttacgggattccattcattgaaacctcagcgaccgacg

SEQ ID NO: 285 - DNA, 51 bp, synthetic construct
  source 1..51: mol_type = other DNA
cgtcggtcaaggacagttgatacaaaacaagcccacgaactgggaccgacg

SEQ ID NO: 286 - DNA, 51 bp, synthetic construct
  source 1..51: mol_type = other DNA
cgtcggtcctatggtgctagtgggaaacaagtgtgatttgccagaccgacg

SEQ ID NO: 287 - DNA, 51 bp, synthetic construct
  source 1..51: mol_type = other DNA
cgtcggtcgagcagattaagcgagtaaaagactcggatgatgtgaccgacg

SEQ ID NO: 288 - DNA, 53 bp, synthetic construct
  source 1..53: mol_type = other DNA
cgtcggtcggcttcctctgtgtatttgccatcaataatagcaagtgaccgacg

SEQ ID NO: 289 - DNA, 51 bp, synthetic construct
  source 1..51: mol_type = other DNA
cgtcggtccagtgccatgagagaccaatacatgaggacaggcggaccgacg

SEQ ID NO: 290 - DNA, 51 bp, synthetic construct
  source 1..51: mol_type = other DNA
cgtcggtctgttggacatactggatacagctggacaagaagaggaccgacg

SEQ ID NO: 291 - DNA, 56 bp, synthetic construct
  source 1..56: mol_type = other DNA
cgtcggtcattcttacagaaaacaagtggttatagatggtgaaacctggaccgacg

SEQ ID NO: 292 - DNA, 51 bp, synthetic construct
  source 1..51: mol_type = other DNA
cgtcggtctccctccctgcccccttaccctccacacccccagggaccgacg

SEQ ID NO: 293 - DNA, 51 bp, synthetic construct
  source 1..51: mol_type = other DNA
cgtcggtcgatcccaccatagaggtgaggcccagtggtagcccgaccgacg

SEQ ID NO: 294 - DNA, 54 bp, synthetic construct
  source 1..54: mol_type = other DNA
cgtcggtcgacaatccagctaatccagaaccactttgtagatgaatgaccgacg

SEQ ID NO: 295 - DNA, 51 bp, synthetic construct
  source 1..51: mol_type = other DNA
cgtcggtctggtggttggagcaggtggtgttgggaaaagcgcagaccgacg

SEQ ID NO: 296 - DNA, 51 bp, synthetic construct
  source 1..51: mol_type = other DNA
cgtcggtcggttcttgctggtgtgaaatgactgagtacaaactgaccgacg

SEQ ID NO: 297 - DNA, 56 bp, synthetic construct
  source 1..56: mol_type = other DNA
cgtcggtctcagcaaagacaagacaggtaagtaacactgaaataaatagaccgacg

SEQ ID NO: 298 - DNA, 56 bp, synthetic construct
  source 1..56: mol_type = other DNA
cgtcggtcggacttagcaagaagttatggaattccttttattgaaacagaccgacg

SEQ ID NO: 299 - DNA, 51 bp, synthetic construct
  source 1..51: mol_type = other DNA
cgtcggtcttgccttctagaacagtagacacaaaacaggctcagaccgacg

SEQ ID NO: 300 - DNA, 55 bp, synthetic construct
  source 1..55: mol_type = other DNA
cgtcggtcctgaagatgtacctatggtcctagtaggaaataaatgtggaccgacg

SEQ ID NO: 301 - DNA, 56 bp, synthetic construct
  source 1..56: mol_type = other DNA
cgtcggtcttttttctttcccagagaacaaattaaaagagttaaggacgaccgacg

SEQ ID NO: 302 - DNA, 56 bp, synthetic construct
  source 1..56: mol_type = other DNA
cgtcggtcttgaaagatatttgtgttactaatgactgtgctataacttgaccgacg

SEQ ID NO: 303 - DNA, 56 bp, synthetic construct
  source 1..56: mol_type = other DNA
cgtcggtcggggagggctttctttgtgtatttgccataaataatactagaccgacg

SEQ ID NO: 304 - DNA, 51 bp, synthetic construct
  source 1..51: mol_type = other DNA
cgtcggtcagtacagtgcaatgagggaccagtacatgaggactgaccgacg

SEQ ID NO: 305 - DNA, 51 bp, synthetic construct
  source 1..51: mol_type = other DNA
cgtcggtctgtctcttggatattctcgacacagcaggtcaagagaccgacg

SEQ ID NO: 306 - DNA, 54 bp, synthetic construct
  source 1..54: mol_type = other DNA
cgtcggtcggattcctacaggaagcaagtagtaattgatggagaaagaccgacg

SEQ ID NO: 307 - DNA, 51 bp, synthetic construct
  source 1..51: mol_type = other DNA
cgtcggtcgcactgtaataatccagactgtgtttctcccttctgaccgacg

SEQ ID NO: 308 - DNA, 56 bp, synthetic construct
  source 1..56: mol_type = other DNA
cgtcggtccattttgtggacgaatatgatccaacaatagaggtaaatcgaccgacg

SEQ ID NO: 309 - DNA, 51 bp, synthetic construct
  source 1..51: mol_type = other DNA
cgtcggtcgcaagagtgccttgacgatacagctaattcagaatgaccgacg

SEQ ID NO: 310 - DNA, 51 bp, synthetic construct
  source 1..51: mol_type = other DNA
cgtcggtcgaatataaacttgtggtagttggagctggtggcgtgaccgacg

SEQ ID NO: 311 - DNA, 56 bp, synthetic construct
  source 1..56: mol_type = other DNA
cgtcggtcacattttcattatttttattataaggcctgctgaaaatgagaccgacg

SEQ ID NO: 312 - DNA, 55 bp, synthetic construct
  source 1..55: mol_type = other DNA
cgtcggtcgtgacagctccatttcttctaaaatagtccattggaaaggaccgacg

SEQ ID NO: 313 - DNA, 53 bp, synthetic construct
  source 1..53: mol_type = other DNA
cgtcggtcggtcatagtaatcaccagtgttctgaatcttgatgtggaccgacg

SEQ ID NO: 314 - DNA, 51 bp, synthetic construct
  source 1..51: mol_type = other DNA
cgtcggtcaactcagccaaagtggcaaatttctcccctccatagaccgacg

SEQ ID NO: 315 - DNA, 54 bp, synthetic construct
  source 1..54: mol_type = other DNA
cgtcggtcctcttttaattgcccgtgatgttccatgtaatactggagaccgacg

SEQ ID NO: 316 - DNA, 55 bp, synthetic construct
  source 1..55: mol_type = other DNA
cgtcggtccagttcagaggatatttaagctcaatgacatctccattcgaccgacg

SEQ ID NO: 317 - DNA, 51 bp, synthetic construct
  source 1..51: mol_type = other DNA
cgtcggtctcgcagtcaacacctacgaaggaaacatcatgaaggaccgacg

SEQ ID NO: 318 - DNA, 51 bp, synthetic construct
  source 1..51: mol_type = other DNA
cgtcggtcagaccgcaccatctggatggttttgggaacgtcaagaccgacg

SEQ ID NO: 319 - DNA, 54 bp, synthetic construct
  source 1..54: mol_type = other DNA
cgtcggtccaatataatgctggaccgccatatagataaatcggtacgaccgacg

SEQ ID NO: 320 - DNA, 51 bp, synthetic construct
  source 1..51: mol_type = other DNA
cgtcggtcggtacctgctcttcttcaatcctgcgctgtagtgtgaccgacg

SEQ ID NO: 321 - DNA, 51 bp, synthetic construct
  source 1..51: mol_type = other DNA
cgtcggtcgcaagagaatgagaatccgcatgccagccctcagggaccgacg

SEQ ID NO: 322 - DNA, 51 bp, synthetic construct
  source 1..51: mol_type = other DNA
cgtcggtcgtcaggggcaatgtgaggctgctatttcctacttagaccgacg

SEQ ID NO: 323 - DNA, 52 bp, synthetic construct
  source 1..52: mol_type = other DNA
cgtcggtcgattggctcgagatatcatgagtgattccaactatggaccgacg

SEQ ID NO: 324 - DNA, 51 bp, synthetic construct
  source 1..51: mol_type = other DNA
cgtcggtcgtcacccacgggaaagtggtgaagatatgtgacttgaccgacg

SEQ ID NO: 325 - DNA, 51 bp, synthetic construct
  source 1..51: mol_type = other DNA
cgtcggtcgtgtgttcacagagacctggccgccaggaacgtgcgaccgacg

SEQ ID NO: 326 - DNA, 51 bp, synthetic construct
  source 1..51: mol_type = other DNA
cgtcggtccctgctgggggcgtgcacactgtcaggtaacccacgaccgacg

SEQ ID NO: 327 - DNA, 51 bp, synthetic construct
  source 1..51: mol_type = other DNA
cgtcggtctgatgacccagctgggaagccacgagaatattgtggaccgacg

SEQ ID NO: 328 - DNA, 51 bp, synthetic construct
  source 1..51: mol_type = other DNA
cgtcggtcagctctgaaagagaggcactcatgtcagaactcaagaccgacg

SEQ ID NO: 329 - DNA, 56 bp, synthetic construct
  source 1..56: mol_type = other DNA
cgtcggtctttacatttttaatgctcctttctttgacagaaaaagcaggaccgacg

SEQ ID NO: 330 - DNA, 56 bp, synthetic construct
  source 1..56: mol_type = other DNA
cgtcggtcccaagagaaaatttagagtttggtaagaatggaatgtgccgaccgacg

SEQ ID NO: 331 - DNA, 56 bp, synthetic construct
  source 1..56: mol_type = other DNA
cgtcggtctgatttcagagaatatgaatatgatctcaaatgggagtttgaccgacg

SEQ ID NO: 332 - DNA, 51 bp, synthetic construct
  source 1..51: mol_type = other DNA
cgtcggtcgtgaccggctcctcagataatgagtacttctacgtgaccgacg

SEQ ID NO: 333 - DNA, 51 bp, synthetic construct
  source 1..51: mol_type = other DNA
cgtcggtcgcaatttaggtatgaaagccagctacagatggtacgaccgacg

SEQ ID NO: 334 - DNA, 52 bp, synthetic construct
  source 1..52: mol_type = other DNA
cgtcggtcctggaagaagtgttcagacaagtctcccaagtaatagaccgacg

SEQ ID NO: 335 - DNA, 51 bp, synthetic construct
  source 1..51: mol_type = other DNA
cgtcggtccctgtttctcggatggatacccattaccatcttgggaccgacg

SEQ ID NO: 336 - DNA, 51 bp, synthetic construct
  source 1..51: mol_type = other DNA
cgtcggtccctcaagtgctcgcagaagcatcggcaagtcaggcgaccgacg

SEQ ID NO: 337 - DNA, 53 bp, synthetic construct
  source 1..53: mol_type = other DNA
cgtcggtcccagtgagcttatttcacacgttcttttctatagggagaccgacg

SEQ ID NO: 338 - DNA, 51 bp, synthetic construct
  source 1..51: mol_type = other DNA
cgtcggtccatggcgaccaggtaggccagggtggagaggggatgaccgacg

SEQ ID NO: 339 - DNA, 51 bp, synthetic construct
  source 1..51: mol_type = other DNA
cgtcggtctggctggaccaagcccatcaccattggcaggcacggaccgacg

SEQ ID NO: 340 - DNA, 51 bp, synthetic construct
  source 1..51: mol_type = other DNA
cgtcggtcagcccatcatctgcaaaaacatcccacgcctagtcgaccgacg

SEQ ID NO: 341 - DNA, 51 bp, synthetic construct
  source 1..51: mol_type = other DNA
cgtcggtcactatccggaacatcctgggggggactgtcttccggaccgacg

SEQ ID NO: 342 - DNA, 51 bp, synthetic construct
  source 1..51: mol_type = other DNA
cgtcggtcgttcaagctgaagaagatgtggaaaagtcccaatggaccgacg

SEQ ID NO: 343 - DNA, 51 bp, synthetic construct
  source 1..51: mol_type = other DNA
cgtcggtcaggggggagcagggctcactccaggtgagtgacctgaccgacg

SEQ ID NO: 344 - DNA, 51 bp, synthetic construct
  source 1..51: mol_type = other DNA
cgtcggtcccttggaactcaaggatgcccaggctgggaaggaggaccgacg

SEQ ID NO: 345 - DNA, 51 bp, synthetic construct
  source 1..51: mol_type = other DNA
cgtcggtccgtgagcgcttcgagatgttccgagagctgaatgagaccgacg

SEQ ID NO: 346 - DNA, 51 bp, synthetic construct
  source 1..51: mol_type = other DNA
cgtcggtctacttctccccctcctctgttgctgcagatccgtggaccgacg

SEQ ID NO: 347 - DNA, 51 bp, synthetic construct
  source 1..51: mol_type = other DNA
cgtcggtcgggagcctcaccacgagctgcccccagggagcactgaccgacg

SEQ ID NO: 348 - DNA, 51 bp, synthetic construct
  source 1..51: mol_type = other DNA
cgtcggtcgaccggcgcacagaggaagagaatctccgcaagaagaccgacg

SEQ ID NO: 349 - DNA, 51 bp, synthetic construct
  source 1..51: mol_type = other DNA
cgtcggtccagctttgaggtgcgtgtttgtgcctgtcctgggagaccgacg

SEQ ID NO: 350 - DNA, 51 bp, synthetic construct
  source 1..51: mol_type = other DNA
cgtcggtcttcctatcctgagtagtggtaatctactgggacgggaccgacg

SEQ ID NO: 351 - DNA, 51 bp, synthetic construct
  source 1..51: mol_type = other DNA
cgtcggtccaccatcatcacactggaagactccaggtcaggaggaccgacg

SEQ ID NO: 352 - DNA, 51 bp, synthetic construct
  source 1..51: mol_type = other DNA
cgtcggtcgttcctgcatgggcggcatgaaccggaggcccatcgaccgacg

SEQ ID NO: 353 - DNA, 51 bp, synthetic construct
  source 1..51: mol_type = other DNA
cgtcggtcgactgtaccaccatccactacaactacatgtgtaagaccgacg

SEQ ID NO: 354 - DNA, 51 bp, synthetic construct
  source 1..51: mol_type = other DNA
cgtcggtcctcatcttgggcctgtgttatctcctaggttggctgaccgacg

SEQ ID NO: 355 - DNA, 51 bp, synthetic construct
  source 1..51: mol_type = other DNA
cgtcggtcgtgtggtggtgccctatgagccgcctgaggtctgggaccgacg

SEQ ID NO: 356 - DNA, 53 bp, synthetic construct
  source 1..53: mol_type = other DNA
cgtcggtcgtgtggagtatttggatgacagaaacacttttcgacagaccgacg

SEQ ID NO: 357 - DNA, 51 bp, synthetic construct
  source 1..51: mol_type = other DNA
cgtcggtccctcctcagcatcttatccgagtggaaggaaatttgaccgacg

SEQ ID NO: 358 - DNA, 51 bp, synthetic construct
  source 1..51: mol_type = other DNA
cgtcggtcgcctctgattcctcactgattgctcttaggtctgggaccgacg

SEQ ID NO: 359 - DNA, 51 bp, synthetic construct
  source 1..51: mol_type = other DNA
cgtcggtcgctcagatagcgatggtgagcagctggggctggaggaccgacg

SEQ ID NO: 360 - DNA, 51 bp, synthetic construct
  source 1..51: mol_type = other DNA
cgtcggtcacggaggttgtgaggcgctgcccccaccatgagcggaccgacg

SEQ ID NO: 361 - DNA, 51 bp, synthetic construct
  source 1..51: mol_type = other DNA
cgtcggtcccgcgccatggccatctacaagcagtcacagcacagaccgacg

SEQ ID NO: 362 - DNA, 51 bp, synthetic construct
  source 1..51: mol_type = other DNA
cgtcggtctgtgggttgattccacacccccgcccggcacccgcgaccgacg

SEQ ID NO: 363 - DNA, 51 bp, synthetic construct
  source 1..51: mol_type = other DNA
cgtcggtcatgttttgccaactggccaagacctgccctgtgcagaccgacg

SEQ ID NO: 364 - DNA, 51 bp, synthetic construct
  source 1..51: mol_type = other DNA
cgtcggtccttcctcttcctacagtactcccctgccctcaacagaccgacg

SEQ ID NO: 365 - DNA, 51 bp, synthetic construct
  source 1..51: mol_type = other DNA
cgtcggtcatctgttcacttgtgccctgactttcaactctgtcgaccgacg

SEQ ID NO: 366 - DNA, 51 bp, synthetic construct
  source 1..51: mol_type = other DNA
cgtcggtcgggcagctacggtttccgtctgggcttcttgcattgaccgacg

SEQ ID NO: 367 - DNA, 51 bp, synthetic construct
  source 1..51: mol_type = other DNA
cgtcggtcccctgtcatcttctgtcccttcccagaaaacctacgaccgacg

SEQ ID NO: 368 - DNA, 51 bp, synthetic construct
  source 1..51: mol_type = other DNA
cgtcggtccctacaccggcggcccctgcaccagccccctcctggaccgacg

SEQ ID NO: 369 - DNA, 51 bp, synthetic construct
  source 1..51: mol_type = other DNA
cgtcggtcagaggctgctccccccgtggcccctgcaccagcaggaccgacg

SEQ ID NO: 370 - DNA, 51 bp, synthetic construct
  source 1..51: mol_type = other DNA
cgtcggtcctgaagacccaggtccagatgaagctcccagaatggaccgacg

SEQ ID NO: 371 - DNA, 51 bp, synthetic construct
  source 1..51: mol_type = other DNA
cgtcggtccctatggaaactgtgagtggatccattggaagggcgaccgacg

SEQ ID NO: 372 - DNA, 51 bp, synthetic construct
  source 1..51: mol_type = other DNA
cgtcggtcgcgtcgagccccctctgagtcaggaaacattttcagaccgacg

SEQ ID NO: 373 - DNA, 51 bp, synthetic construct
  source 1..51: mol_type = other DNA
cgtcggtccgggtcactgccatggaggagccgcagtcagatccgaccgacg

SEQ ID NO: 374 - DNA, 51 bp, synthetic construct
  source 1..51: mol_type = other DNA
cgtcggtctccccacttttcctcttgcagcagccagactgcctgaccgacg

SEQ ID NO: 375 - DNA, 53 bp, synthetic construct
  source 1..53: mol_type = other DNA
cgtcggtcgcttatggggatcaagtaagtcatgttggcaataatggaccgacg

SEQ ID NO: 376 - DNA, 51 bp, synthetic construct
  source 1..51: mol_type = other DNA
cgtcggtcgagtggatgggtaaaacctatcatcataggtcgtcgaccgacg

SEQ ID NO: 377 - DNA, 51 bp, synthetic construct
  source 1..51: mol_type = other DNA
cgtcggtcgagaagccattatctgcaaaaatatcccccggcttgaccgacg

SEQ ID NO: 378 - DNA, 51 bp, synthetic construct
  source 1..51: mol_type = other DNA
cgtcggtcggcaccatacgaaatattctgggtggcacggtcttgaccgacg

SEQ ID NO: 379 - DNA, 56 bp, synthetic construct
  source 1..56: mol_type = other DNA
cgtcggtcgttgaggagttcaagttgaaacaaatgtggaaatcaccaagaccgacg

(19) United States
(12) Patent Application Publication
(10) Pub. No.: US 2025/0027076 A1
(43) Pub. Date: Jan. 23, 2025

(54) LIQUID-PHASE HYBRID CAPTURE METHOD AND LIQUID-PHASE HYBRID CAPTURE KIT

(71) Applicant: NANODIGMBIO (NANJING) BIOTECHNOLOGY CO., LTD., Nanjing, Jiangsu (CN)

(72) Inventors: Liping YU, Nanjing, Jiangsu (CN); Biao WANG, Nanjing, Jiangsu (CN); Changquan JI, Nanjing, Jiangsu (CN); Qiang WU, Nanjing, Jiangsu (CN)

(73) Assignee: NANODIGMBIO (NANJING) BIOTECHNOLOGY CO., LTD., Nanjing, Jiangsu (CN)

(21) Appl. No.: 18/280,967

(22) PCT Filed: Aug. 11, 2022
(86) PCT No.: PCT/CN2022/111685

§ 371 Date: Sep. 8, 2023

(30) Foreign Application Priority Data
May 16, 2022 (CN) 202210531282.2

(51) Int. Cl. C12N 15/10

(52) U.S. Cl. CPC C12N 15/1093

(57) ABSTRACT

The present disclosure provides a liquid-phase hybrid capture method, including the steps of probe design and synthesis, library construction and hybrid capture. The hybrid capture is optimized, and the method of the present disclosure can be used for the construction of various nucleic acid sample capture libraries from different sources, effectively increasing the capture efficiency, shortening the experimental time, and simplifying the experimental process, and the method is used for target region (Panel) capture.

### FIELD

The present disclosure relates to the field of genetic detection, in particular to a liquid-phase hybrid capture method.

### BACKGROUND

A nucleic acid sequence is a carrier of life information, while a high-throughput sequencing technology has become one of the core technologies in the biological and medical fields. High-throughput sequencing produces a large amount of data, not all of which are target sequences for research or detection. Although the cost of sequencing has been significantly reduced, due to the high volume of whole genome sequencing data, the cost is still high, and a solution to this problem is to change whole genome sequencing into a targeted enrichment technique. A target region-enriched NGS sequencing technique will ignore information from regions of non-interest in a genome and amplify signals from a target region in the genome, which can save the sequencing cost and the sequencing time.

Targeted enrichment is mainly divided into multiplex PCR amplification and targeted capture based on different enrichment principles. The latter is a probe-based liquid-phase hybrid capture technology, is a mainstream at present, and has the advantages of low probe design difficulty and high probe fault tolerance. The liquid-phase hybrid capture technology is that a biotin-labeled probe specifically binds to a target region in a solution, and target fragments captured by the probe are enriched by streptavidin magnetic beads. During this process, the probe labeled with biotin and liquid phase reaction conditions of hybrid capture have a significant impact on the capture efficiency of this system. For a large target region, the hybrid capture efficiency is higher, for example, a whole exon target region (Panel, also known as a capture region) has an on-target rate of 80% or more; however, for some small target regions (Panels), the on-target rate is relatively low; for example, a small target region of 10 kb or below has an on-target rate of a single digit or below.

The selection of a probe sequence length has various considerations: first, the probe length should ensure that in a specific hybridization system, under different sequence base compositions, the hybridization annealing temperature is appropriate, and the binding ability and specificity of the probe with a target sequence are optimal: secondly, it should be ensured that when there is a certain degree of mismatch between sequences of the probe and the target sequence, the hybridization annealing temperature does not decrease significantly; and finally, the longer the probe, the more difficult to synthesize it, and the more difficult to ensure the quality of synthesis. Currently, based on the above considerations, the probe sequence length is generally 40-120 nt, while the mainstream probe length is 120 nt, and is modified (such as biotin), and its modified group can bind to a corresponding affinity medium to complete the “capture” of the target sequence. The forms of the probe include single-stranded DNA, double-stranded DNA, single-stranded RNA, double-stranded RNA, and the like.

Currently, a second generation sequencing technology is the most widely used high-throughput sequencing technology, with bi-directional 150 bp being a more mainstream sequencing reading mode. The average insert fragment length of a sequencing library is also 100-400 bp. The middle part of an excessively long insert fragment cannot be read, and the excessively long fragment also poses a challenge to multiple PCR amplification steps in the sequencing process. In addition, for samples with a short original length, such as FFPE and extracellular free nucleic acid, it is impossible to prepare a library with longer insert fragments. Then, one library molecule typically can only bind to 1-2 probes during hybrid capture, which also means that the probability of probe detachment increases and the recovery rate of the target sequence decreases. For example, a target sequence of 120 bp in length can only bind to one probe completely at most, and even if the target sequence can bind to two probes, the two probes can only be partially bound. In order to increase the binding capacity and probability of probes, the probes may be shortened, or an imbricated design strategy may be adopted, i.e., the probes are overlapped with each other so that different target sequence fragments have a higher probability of more complete binding to the probes. However, even probes which are overlapped with each other cannot completely bind to the same target fragment simultaneously.

For a sequencing library subjected to PCR amplification, there are multiple copies in each target fragment, and therefore a lower recovery rate can also ensure that most of the original target fragments have captured copies. And the hybrid capture technology typically targets regions of 5 kb or more, while for inherent non-specific capture, compression can be performed by a variety of means, with an on-target rate (a proportion of a target sequence in all captured sequences) being guaranteed to a certain extent. However, current mainstream probes and hybrid capture systems do not provide a satisfactory recovery efficiency and on-target rate for a sequencing library that has short insert fragments, or is not subjected to PCR amplification, and an application requirement with a low proportion of target regions in total regions.

In addition, the liquid-phase hybrid capture process is very time-consuming, taking 2-4 days from a nucleic acid sample to capture library obtaining: meanwhile, hybrid capture involves a large number of reagents, is an extremely cumbersome operation process, and has high technical requirements for operators. A problem in any link of the process will affect the performance of the capture library. These links become critical technical bottlenecks that restrict the development of liquid-phase hybrid capture.

The liquid-phase hybrid capture technology is widely used in cancer tumor mutation gene detection, copy number variation, and methylation status analysis. At present, many products are applied to gene detection and clinical application research in the market. However, with the rise in the popularity of early screening of tumors and MRD, higher requirements are put forward for the liquid-phase hybrid capture technology. For example, for a solid tumor MRD detection technology, primary tumor tissue is first sequenced to identify patient-specific genomic variation maps, and then a target region is designed for personalized ctDNA detection analysis. This requires higher requirements for a hybrid capture system in terms of compatibility with small target regions, ease of operation, degeneracy of experimental processes, and degree of automation.

Therefore, developing a probe with high recovery efficiency and a high on-target rate, as well as a liquid-phase hybrid capture system with high capture efficiency, uniformity, stability; and easy operation, fewer types of reagents, and short time consumption is a solution to solve the problems in the current market.

### SUMMARY

An objective of the present disclosure is to provide a liquid-phase hybrid capture method directed against the limitations in the prior art such as a complex hybridization enrichment solution and a long reaction time.

To achieve the above objective, the present disclosure provides the following technical solution:

A liquid-phase hybrid capture method comprises the following steps of:
(1) probe design: designing a pool of probes based on nucleic acid target regions, wherein each probe includes a probe binding sequence complementarily pairing with another probe, and a target specific sequence complementarily pairing with a nucleic acid target sequence, synthesizing each oligonucleotide based on corresponding sequence, and modifying a 5′ or 3′ end with a biomarker;
(2) library construction;
(3) hybrid capture
i. configuring a hybridization system: wherein the hybridization system includes the probes; and
ii. carrying out a hybridization reaction: placing the hybridization system at 57-63°° C. for hybridization for 1-2 h;
(4) product capture: after the hybridization reaction is completed, adding streptavidin magnetic beads to the reaction system for hybrid capture;
(5) product elution: after capture is completed, separately washing the captured product once with an elution buffer I, an elution buffer II and an elution buffer III; and
(6) product amplification and purification: after the washing is completed, adding a PCR reaction system for a PCR amplification procedure, and after the reaction is completed, performing purification by using magnetic beads.

Preferably, the probe binding sequence includes a first probe binding sequence and a second probe binding sequence.

More preferably, a 5′ end of each probe has a first probe binding sequence complementarily pairing with a 3′ end of another probe, and a 3′ end of each probe has a second probe binding sequence complementarily pairing with a 5′ end of another probe. The probe binding sequence has a length of k, and has an annealing temperature that is less than the annealing temperature for binding of the probe to a target sequence, and k is selected so that a minimum value of the number of occurrences of all sequence combinations in the sum sequence length is achieved.

Preferably, the probe binding sequence is 8-30 nt in length.

Preferably, the target specific sequence is 20-80 nt in length.

More preferably, the first probe binding sequence complementarily pairing with another probe at the 5′ end of each probe is 20-80 nt in length, and the second probe binding sequence complementarily pairing with another probe at the 3′ end of each probe is 8-30 nt in length.

Preferably, the biomarker may be amino acid, biotin, polypeptide tag, heparin, polysaccharide or lipid.

Preferably, the hybridization system includes 2-10 fmol of the probe, 1×Hyb Buffer, 1×Enhance, lug of Human Cot-1, and 100 pmmol of Blocker.

More preferably, the probe in the hybridization system has a concentration of 6 fmol.

Preferably, the hybridization reaction is carried out by denaturation at 95° C. for 2 min, and hybridization at 60° C. for 1 h.

Preferably, the product capture is carried out at 58° C. for 20 min.

Preferably, the PCR reaction system includes 2×HiFi PCR Master Mix, 5 μL of Index Primer Mix and 20 μL of TE.

Preferably, the nucleic acid is from fresh tissue, frozen tissue, paraffin embedded tissue, hydrothorax and ascites, plasma or exfoliated tumor cell tissue.

Preferably, the nucleic acid is plasma free DNA, genomic DNA or RNA.

Preferably, the library construction is to construct a DNA library based on nucleic acid fragment size of 200-250 bp.

Preferably, the library construction includes reverse transcription, first strand synthesis and second strand synthesis of a RNA sample.

Preferably, the library construction includes end repair and adapter ligation of nucleic acid fragments.

Preferably, formula of the elution buffer I is 5×SSPE, and 0.5-5% of SDS; formula of the elution buffer II is 2×SSPE, and 0.05-0.5% of SDS; and formula of the elution buffer III is 0.1×SSPE, and 0.005%-0.05% of SDS.

The present disclosure also provides a design method for the pool of probes, including the following steps of:
(1) generating sequence information by inputting pre-capture library information and design parameters, wherein the sequence information includes total sequence information and target sequence information, and the design parameters include an annealing temperature range, and a sequence length range for binding of probes to a target sequence;
(2) counting the number of occurrences of all sequence combinations with a length of k in a plus strand and a complementary strand in the sum sequence, wherein k is less than the minimum value in the sequence length range of binding regions of the probes to target sequences;
(3) selecting a probe binding sequence in which probes are complementary paired, wherein the probe binding sequence has a length of k, and has an annealing temperature that is less than the annealing temperature for binding of the probes to the target sequence, and occurs less frequently in the sum sequence, preferably, the number of occurrences is less than 5% of the average;
(4) selecting a target specific sequence in which the probes bind to a nucleic acid target sequence, wherein an ith target sequence is selected, i having an initial value equal to 1; the target specific sequence in which the probes bind to the nucleic acid target sequence is then selected starting from an nth base of the selected target sequence, n having an initial value equal to 1;
(5) adding the probe binding sequence to a 5′ end of the target specific sequence, and adding a reverse complementary sequence of the probe binding sequence to a 3′ end of the target specific sequence; and
(6) outputting all probe sequences.

Preferably, if the specificity of the target specific sequence in which the probes bind to the nucleic acid target sequence is evaluated as high specificity, the target specific sequence is placed in the pool of probes, and spaced by adding a number m1 to the base n; and if the specificity of the target specific sequence in which the probes bind to the nucleic acid target sequence is evaluated as low specificity, the target specific sequence is not placed in the pool of probes, and spaced by adding a number m2 to the base n;

Wherein the number m1 is greater than or equal to the length of each probe and the length of the target specific sequence: and the number m2 is less than or equal to a minimum value of a length range of each probe and a length range of the target specific sequence.

Preferably, selecting the target specific sequence in which the probes bind to the nucleic acid target sequence includes the steps of: selecting a next target specific sequence when n is less than the length of the ith target sequence; and selecting the ith target specific sequence when n is greater than or equal to the length of the ith target sequence. After selection of the target specific sequence of the ith target sequence is finished, the above target specific sequence selection is performed on an i+1th target sequence until the target specific sequence selection is completed for all target sequences.

In addition, the present disclosure also provides a liquid-phase hybrid capture kit, comprising the following components: probes, a hybridization reaction solution, an elution buffer, and nucleic acid purification magnetic beads; wherein each probe includes a probe binding sequence complementarily pairing with another probe, and a target specific sequence complementarily pairing with a nucleic acid target sequence.

Preferably, the kit further includes an end repair enzyme mixture, an end repair reaction buffer, a molecular tag-containing adapter, library amplification primers, a PCR premix, an adapter blocker, a DNA blocker, a hybridization enhancer, a magnetic bead wash buffer, and capture library PCR primers.

In another aspect, the present disclosure also provides use of a liquid-phase hybrid capture method in genomic target region capture.

Preferably, the method is applied to low-frequency mutation detection, chromosome copy number variation analysis, insertion/deletion, and fusion gene detection in nucleic acid fragments; or is used for targeted metagenomic next-generation sequencing (mNGS), and epidemiological detection of pathogens.

Compared with the prior art, the method of the present disclosure has the beneficial effects that:
(1) the processes of the hybrid capture system are optimally designed, taking into account the application requirements of an automated workstation, the connection between steps is high, the disadvantage that the conventional hybrid capture process is cumbersome is overcome, the manual operation friendliness is high, and the implementability of being applied to the automated workstation is high.
(2) The hybrid capture system has high capture efficiency, and flank sequences of the probes are complementary paired, which can significantly improve the binding ability of the probes to a target, improve the hybrid capture efficiency of the target region, and improve the overall coverage uniformity and stability.

### BRIEF DESCRIPTION OF THE DRAWINGS

The accompanying drawings forming part of this application are intended to provide a further understanding of the present disclosure. The illustrative embodiments of the present disclosure and explanations thereof are intended to explain the present disclosure and do not constitute an improper limitation of the present disclosure. In the accompanying drawings:

FIG. 1 is a structural schematic diagram of probes according to the present disclosure, wherein each probe is mainly composed of 4 parts: a P-Cap region complementary to target genes, a P-L region at a 3′ end, and a P-R region at a 5′ end, wherein the 5′ end of each probe is labeled with biotin, and the P-L and the P-R have a sequence complementary to each other.

FIG. 2 is a graph comparing a flow of a conventional hybrid capture system and a flow of a hybrid capture system of the present disclosure.

FIG. 3 is an experimental protocol for different types of samples.

FIG. 4 is a structural schematic diagram of a conventional probe of 120 nt, a short probe used in the prior art, and the probe of the present disclosure, wherein T represents a target fragment of a sample nucleic acid, and P represents the probe.

FIG. 5 is an experimental result of hybrid capture library NGS for the conventional probes of 120 nt, the short probes used in the prior art, and the probes of the present disclosure.

FIG. 6 shows the capture effect of the conventional probes of 120 nt and the probes of the present disclosure for a PCR-free library.

FIG. 7 shows a concentration test result of the probes according to the present disclosure.

FIG. 8 shows a hybridization temperature test result of the probes according to the present disclosure.

FIG. 9 shows a hybridization time test result of the probes according to the present disclosure.

### DETAILED DESCRIPTION OF THE EMBODIMENTS

The present disclosure will be further described below with reference to the accompanying drawings and specific embodiments. The protection content of the present disclosure is not limited to the following embodiments. It should also be understood that the terms used in the embodiments of the present disclosure are intended to describe specific embodiments, not to limit the scope of protection of the present disclosure, and are not unique limitations. Changes and advantages that can be contemplated by those skilled in the art without departing from the spirit and scope of the inventive concept are included in the present disclosure, and the appended claims and any equivalents thereof are the scope of protection of the present disclosure.

All technical and scientific terms used herein have the same meaning as that commonly understood by those skilled in the art to which the present disclosure belongs. In other cases, certain terms used herein will have their meanings set forth in the specification. Experimental methods in which specific conditions are not indicated in the following embodiments are within the general knowledge and common general knowledge of those skilled in the art. Reagents used in the embodiments, unless otherwise specified, were purchased from reagent companies provided that the experimental requirements were met. The embodiments in this application and the features in the embodiments can be combined with each other.

The features and advantages of the present disclosure will be further understood from the following detailed description in conjunction with the accompanying drawings. The embodiments provided are merely illustrative of the method of the present disclosure, and are not intended to limit the rest of the contents of the present disclosure in any way.

The present disclosure provides a set of probes for nucleic acid capture, wherein the probes are designed separately for a positive sense strand and a negative sense strand of a target region, the probes for the positive sense strand and the probes for the negative sense strand are arranged in a non-overlapping arrangement, and a 3′ or 5′ end of each probe is modified with biotin which can bind to streptavidin magnetic beads.

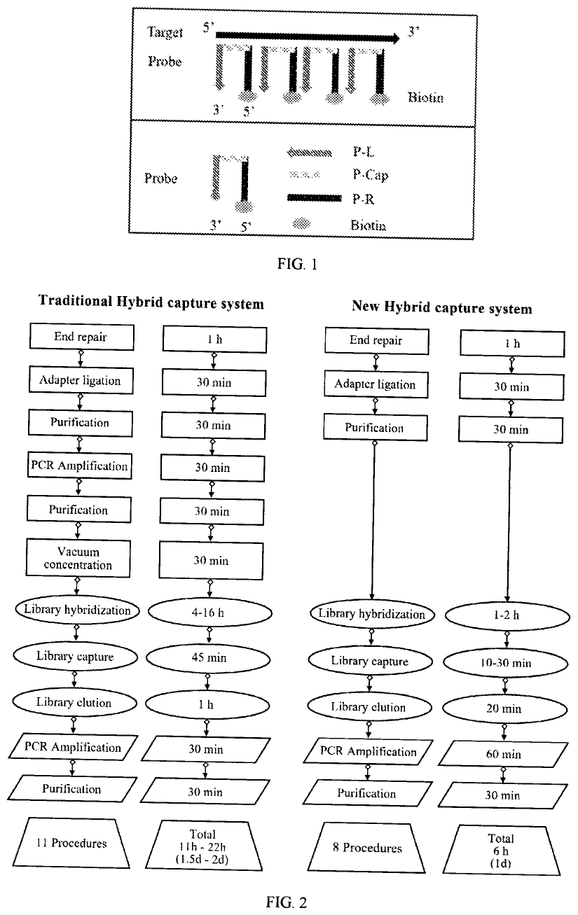

Each probe is primarily composed of three parts, wherein a middle segment is a target sequence binding segment, 5′ and 3′ segments are stability enhancing segments, the 5′ end segment of one probe can be complementarily paired with the 3′ end segment of another probe, and the 3′ end segment of one probe can be complementarily paired with the 5′ end segment of another probe. Fragments where the probes are complementarily paired are P-L and P-R fragments, respectively, with biotin modified at a 3′ end of L or at a 5′ end of R, and the biotin can bind to streptavidin on magnetic beads, and a fragment where the probes are complementarily paired with the target region is a P-Cap fragment with a P-Cap length of 20-80 nt (FIG. 1).

The probe design method is as follows:
probes are designed based on the positions of genes to be detected, namely if the probes are designed for mutation, insertion or deletion mutation, a region covering the corresponding fragment is selected to design the probes: and if the probes are designed for fusion genes, genes on both sides of a fusion gene breakpoint are selected to design the probes;
if capture of a sense strand is desired, a capture probe will be designed for the sense strand;
if capture of an antisense strand is desired, a capture probe will be designed for the antisense strand; and
by software analysis, hazardous probes are knocked out, and the hazardous probes will lead to severe off-target of the entire hybrid capture system, resulting in a reduced on-target rate, low target region capture efficiency, and poor coverage uniformity.

The present disclosure also provides a system for construction of a target library from a nucleic acid sample (see FIG. 2), wherein a specific process is as follows:
the nucleic acid sample includes a DNA sample including plasma free DNA (cfDNA), genomic DNA (gDNA), an FFPE sample, a viral or bacterial genome sample, and the like; and a RNA sample including a fresh tissue sample, an FFPE sample, a viral or bacterial genome sample, and the like.

For the cfDNA sample, without fragmenting, library construction can be performed directly;

for a complete genome sample, physical fragmenting is needed to be performed to fragment genomic DNA to about 200-250 bp;
for the RNA sample, reverse transcription, first strand synthesis and second strand synthesis are needed to be performed; and
the fragmented sample is subjected to end repair and adapter ligation, and the ligation product is purified, and the purified product is directly subjected to hybrid capture, wherein a hybrid capture solution is related to an adapter used, multi-library mixed hybridization can be performed by using a full-UDI adapter module, and the hybridization product uses Primer Mix to perform PCR amplification on the mixed hybridization captured library; if a truncated molecular tag adaptor module is used, only a single sample can be hybridized, the molecular tag-containing adaptor module can perform low-frequency mutation detection on the sample, and hybridization ambiguity and background noise introduced by the PCR amplification are filtered out through consistent sequence analysis. Here an adapter module compatible with both Illumina and MGI sequencing platforms is used to construct a DNA library suitable for different sequencing platforms.

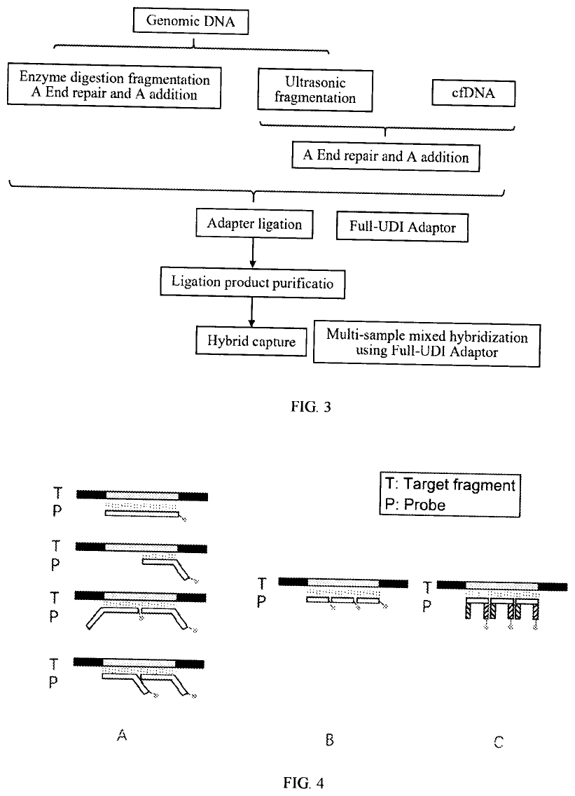

The adapter ligation product is directly used for configuring a hybrid capture reaction system without vacuum concentration, or hybrid capture can be performed directly with the adapter ligation product together with the purification magnetic beads from the previous step; and
a hybridization system uses specific probes designed in this project, so rapid hybridization can be performed. The hybridization time is 1-2 hours, and the capture time is 20 minutes, which shortens the hybrid capture time. The hybrid capture library is enriched by the PCR amplification. A PCR amplification solution in this step is related to the adaptor module used. The PCR amplification is performed in combination with primers containing a Barcode sequence when the molecular tag adaptor module is used, and the targetedly enriched DNA library is amplified in combination with Primer Mix if the full-length adaptor module is used (see FIG. 3).

The hybrid capture time selected for this system is 1 h to 16 h, with the most preferred capture time being 1 h.

The hybrid capture temperature selected for this system is 59-61° C., and the optimal capture temperature is about 60° C., and temperature selection is related to a probe length, the GC content of a target region, and the hybrid capture time.

The construction of the hybrid capture library in this system takes a total of 6 hours from a sample to capture library obtaining, which greatly shortens the operation time of the whole process while simplifying the operation steps compared with the traditional 2-4 days.

The present disclosure also provides hybrid capture reagent components and a use method thereof, wherein the specific content is as follows:
the adapter ligation product is purified by using 2×Beads, and the purified product is treated by using a magnetic bead wash buffer configured in a kit, wherein the magnetic bead wash buffer is 4 mL of acetonitrile added into 1 mL of H₂O.

The reagents used in the hybrid capture reaction system are detailed in Table 1.

TABLE 1

| Reagent | Brand |
|---|---|
| 2 × Hyb Buffer | |
| 0.01-1% BSA | Sigma |
| 0.01-1% Ficoll | Sigma |
| 0.01-1% PVP-2 | Sigma |
| 0.01-0.5 M sodium citrate | Sigma |
| 0.1-10M NaCl | Invitrogen |
| Enhancer: 5 × formamide solution | Thermo |
| Human Cot-1 1 μg/μL | Thermo |
| Blocker 100 nmol | Nanodigmbio |
| pH 6.0-8.0 | |
| Probe concentration: 2-10 fmol | Nanodigmbio |

The hybridization system involves a total of 3 elution buffers, which are an elution buffer I, an elution buffer II and an elution buffer III, respectively, and formulas of the three elution buffers are shown in Table 2.

TABLE 2

| | |
|---|---|
| Elution buffer I | 5 × SSPE (Sigma), 1% of SDS |
| | (Sigma) |
| Elution buffer II | 2 × SSPE, 0.1% of SDS |
| Elution buffer III | 0.1 × SSPE, 0.01% of SDS |

A structural schematic diagram of the probes of the present disclosure, a conventional probe of 120 nt and a short probe is shown in FIG. 4. The effect of the probes of the present disclosure is compared with the effect of a probe commonly used in the prior art in the following Examples 1-3, and the probes of the present disclosure are referred to NC probes.

The amount of each component configured in the kit of the present disclosure can be determined by those skilled in the art according to a predetermined purpose, and kits including any configured amount of the above components are within the protection scope of the present disclosure.

The kit of the present disclosure may also include an instruction for use. The “instruction for use” typically includes a definite recitation describing a technique employed when the components of the kit are used to achieve a desired result. Optionally, the kit may also contain other applicable components, such as a diluent, a buffer, a pharmaceutically acceptable carrier, or other applicable accessories that will be readily recognized by those skilled in the art.

### Example 1: Comparison of a Hybrid Capture Effect of a Conventional Probe of 120 Bases With That of a Short Probe

In this Example, the pre-capture library is a human plasma free DNA library derived from fragmentation and release of human genomic DNA into a blood circulation system, i.e., a sum sequence is the entire human genomic sequence. The target sequences given are located in the regions shown in Table 3, containing a series of high-frequency somatic mutation sites associated with tumors.

TABLE 3
Locations of target sequences on hg19 version of human genome

| | Target region | Target region | |
|---|---|---|---|
| | starting point | endpoint | |
| Chromosome | coordinate | coordinate | Gene name |
| chr1 | 115252204 | 115252205 | NRAS |
| chr1 | 115256518 | 115256533 | NRAS |
| chr1 | 115258730 | 115258752 | NRAS |
| chr2 | 209113106 | 209113193 | IDH1 |
| chr12 | 25378561 | 25378563 | KRAS |
| chr12 | 25378647 | 25378648 | KRAS |
| chr12 | 25380275 | 25380286 | KRAS |
| chr12 | 25398255 | 25398296 | KRAS |
| chr12 | 112888139 | 112888212 | PTPN11 |
| chr12 | 112926852 | 112926909 | PTPN11 |
| chr13 | 28592620 | 28592654 | FLT3 |
| chr13 | 28602329 | 28602330 | FLT3 |
| chr13 | 28608244 | 28608342 | FLT3 |
| chr13 | 28610138 | 28610139 | FLT3 |
| chr15 | 90631837 | 90631939 | IDH2 |
| chr17 | 7573931 | 7574027 | TP53 |
| chr17 | 7577022 | 7577146 | TP53 |
| chr17 | 7577515 | 7577606 | TP53 |
| chr17 | 7578187 | 7578293 | TP53 |
| chr17 | 7578362 | 7578559 | TP53 |
| chr17 | 7579358 | 7579474 | TP53 |
| chr17 | 7579882 | 7579883 | TP53 |

The total length of the target sequence is only 1.2 kb, and if coverage is conducted with a conventional probe of 120 nt, 44 probes are required, wherein the 44 conventional probes of 120 nt are shown in Table 4. Hybrid capture is performed with NadPrep® hybrid capture reagents in this experiment, and the resulting capture library is sequenced on an Illumina Novaseq6000. In the sequencing data, 99.9% of the sequences can be mapped to a human reference genome on average, wherein 11.7% of the sequences is located in the target region on average, and the on-target rate of the probes of 120 nt is too low to meet the requirements.

TABLE 4
Conventional probe of 120 nt covering the target region in Table 3

| | Sequence | | |
|---|---|---|---|
| | name | Sequence 5′-3′ | Modification |
| SEQ ID | NRAS-1 | /biotin/TGCTGAAAGCTGTACCATACCTGTCTGGTCTTGGCT | 5′ biotin |
| NO. 1 | | GAGGTTTCAATGAATGGAATCCCGTAACTCTTGGCCAGTT | |
| | | CGTGGGCTTGTTTTGTATCAACTGTCCTTGTTGGCAAATCA | |
| | | CAC | |
| SEQ ID | NRAS-2 | /biotin/TTTCAATGAATGGAATCCCGTAACTCTTGGCCAGTT | 5′ biotin |
| NO. 2 | | CGTGGGCTTGTTTTGTATCAACTGTCCTTGTTGGCAAATCA | |
| | | CACTTGTTTCCCACTAGCACCATAGGTACATCATCCGAGTC | |
| | | TT | |
| SEQ ID | NRAS-3 | /biotin/GCTATTATTGATGGCAAATACACAGAGGAAGCCTTC | 5′ biotin |
| NO. 3 | | GCCTGTCCTCATGTATTGGTCTCTCATGGCACTGTACTCTT | |
| | | CTTGTCCAGCTGTATCCAGTATGTCCAACAAACAGGTTTCA | |
| | | CC | |
| SEQ ID | NRAS-4 | /biotin/ATTGGTCTCTCATGGCACTGTACTCTTCTTGTCCAGC | 5′ biotin |
| NO. 4 | | TGTATCCAGTATGTCCAACAAACAGGTTTCACCATCTATA | |
| | | ACCACTTGTTTTCTGTAAGAATCCTGGGGGTGTGGAGGGT | |
| | | AAG | |
| SEQ ID | NRAS-5 | /biotin/TACCACTGGGCCTCACCTCTATGGTGGGATCATATT | 5′ biotin |
| NO. 5 | | CATCTACAAAGTGGTTCTGGATTAGCTGGATTGTCAGTGC | |
| | | GCTTTTCCCAACACCACCTGCTCCAACCACCACCAGTTTGT | |
| | | ACT | |
| SEQ ID | NRAS-6 | /biotin/CTCACCTCTATGGTGGGATCATATTCATCTACAAAG | 5′ biotin |
| NO. 6 | | TGGTTCTGGATTAGCTGGATTGTCAGTGCGCTTTTCCCAAC | |
| | | ACCACCTGCTCCAACCACCACCAGTTTGTACTCAGTCATTT | |
| | | CA | |
| SEQ ID | KRAS-1 | /biotin/TTTATTTCAGTGTTACTTACCTGTCTTGTCTTTGCTGA | 5′ biotin |
| NO. 7 | | TGTTTCAATAAAAGGAATTCCATAACTTCTTGCTAAGTCCT | |
| | | GAGCCTGTTTTGTGTCTACTGTTCTAGAAGGCAAATCACAT | |
| SEQ ID | KRAS-2 | /biotin/TTTCAATAAAAGGAATTCCATAACTTCTTGCTAAGT | 5′ biotin |
| NO. 8 | | CCTGAGCCTGTTTTGTGTCTACTGTTCTAGAAGGCAAATCA | |
| | | CATTTATTTCCTACTAGGACCATAGGTACATCTTCAGAGTC | |
| | | CT | |
| SEQ ID | KRAS-3 | /biotin/AGCCTGTTTTGTGTCTACTGTTCTAGAAGGCAAATC | 5′ biotin |
| NO. 9 | | ACATTTATTTCCTACTAGGACCATAGGTACATCTTCAGAGT | |
| | | CCTTAACTCTTTTAATTTGTTCTCTGGGAAAGAAAAAAAAG | |
| | | TT | |
| SEQ ID | KRAS-4 | /biotin/AGTATTATTTATGGCAAATACACAAAGAAAGCCCTC | 5′ biotin |
| NO. 10 | | CCCAGTCCTCATGTACTGGTCCCTCATTGCACTGTACTCCT | |
| | | CTTGACCTGCTGTGTCGAGAATATCCAAGAGACAGGTTTC | |
| | | TCC | |
| SEQ ID | KRAS-5 | /biotin/ACTGGTCCCTCATTGCACTGTACTCCTCTTGACCTGC | 5′ biotin |
| NO. 11 | | TGTGTCGAGAATATCCAAGAGACAGGTTTCTCCATCAATT | |
| | | ACTACTTGCTTCCTGTAGGAATCCTGAGAAGGGAGAAACA | |
| | | CAG | |
| SEQ ID | KRAS-6 | /biotin/TTTACCTCTATTGTTGGATCATATTCGTCCACAAAAT | 5′ biotin |
| NO. 12 | | GATTCTGAATTAGCTGTATCGTCAAGGCACTCTTGCCTACG | |
| | | CCACCAGCTCCAACTACCACAAGTTTATATTCAGTCATTTT | |
| | | C | |
| SEQ ID | KRAS-7 | /biotin/GTCCACAAAATGATTCTGAATTAGCTGTATCGTCAA | 5′ biotin |
| NO. 13 | | GGCACTCTTGCCTACGCCACCAGCTCCAACTACCACAAGT | |
| | | TTATATTCAGTCATTTTCAGCAGGCCTTATAATAAAAATAA | |
| | | TGA | |
| SEQ ID | PTPN11-1 | /biotin/TTTCCAATGGACTATTTTAGAAGAAATGGAGCTGTC | 5′ biotin |
| NO. 14 | | ACCCACATCAAGATTCAGAACACTGGTGATTACTATGACC | |
| | | TGTATGGAGGGGAGAAATTTGCCACTTTGGCTGAGTTGGT | |
| | | CCAG | |
| SEQ ID | PTPN11-2 | /biotin/ACTGGTGATTACTATGACCTGTATGGAGGGGAGAAA | 5′ biotin |
| NO. 15 | | TTTGCCACTTTGGCTGAGTTGGTCCAGTATTACATGGAACA | |
| | | TCACGGGCAATTAAAAGAGAAGAATGGAGATGTCATTGA | |
| | | GCTT | |
| SEQ ID | PTPN11-3 | /biotin/TCATGATGTTTCCTTCGTAGGTGTTGACTGCGATATT | 5′ biotin |
| NO. 16 | | GACGTTCCCAAAACCATCCAGATGGTGCGGTCTCAGAGGT | |
| | | CAGGGATGGTCCAGACAGAAGCACAGTACCGATTTATCTA | |
| | | TAT | |
| SEQ ID | PTPN11-4 | /biotin/GAGGTCAGGGATGGTCCAGACAGAAGCACAGTACC | 5′ biotin |
| NO. 17 | | GATTTATCTATATGGCGGTCCAGCATTATATTGAAACACTA | |
| | | CAGCGCAGGATTGAAGAAGAGCAGGTACCAGCCTGAGGG | |
| | | CTGGC | |
| SEQ ID | FLT3-1 | /biotin/TAGGAAATAGCAGCCTCACATTGCCCCTGACAACAT | 5′ biotin |
| NO. 18 | | AGTTGGAATCACTCATGATATCTCGAGCCAATCCAAAGTC | |
| | | ACATATCTTCACCACTTTCCCGTGGGTGACAAGCACGTTCC | |
| | | TGG | |
| SEQ ID | FLT3-2 | /biotin/TTGCCCCTGACAACATAGTTGGAATCACTCATGATA | 5′ biotin |
| NO. 19 | | TCTCGAGCCAATCCAAAGTCACATATCTTCACCACTTTCCC | |
| | | GTGGGTGACAAGCACGTTCCTGGCGGCCAGGTCTCTGTGA | |
| | | ACA | |
| SEQ ID | FLT3-3 | /biotin/GTTACCTGACAGTGTGCACGCCCCCAGCAGGTTCAC | 5′ biotin |
| NO. 20 | | AATATTCTCGTGGCTTCCCAGCTGGGTCATCATCTTGAGTT | |
| | | CTGACATGAGTGCCTCTCTTTCAGAGCTGTCTGCTTTTTCT | |
| | | GT | |
| SEQ ID | FLT3-4 | /biotin/CCCCCAGCAGGTTCACAATATTCTCGTGGCTTCCCA | 5′ biotin |
| NO. 21 | | GCTGGGTCATCATCTTGAGTTCTGACATGAGTGCCTCTCTT | |
| | | TCAGAGCTGTCTGCTTTTTCTGTCAAAGAAAGGAGCATTA | |
| | | AAA | |
| SEQ ID | FLT3-5 | /biotin/CATTCCATTCTTACCAAACTCTAAATTTTCTCTTGGA | 5′ biotin |
| NO. 22 | | AACTCCCATTTGAGATCATATTCATATTCTCTGAAATCAAC | |
| | | GTAGAAGTACTCATTATCTGAGGAGCCGGTCACCTGTACC | |
| | | AT | |
| SEQ ID | FLT3-6 | /biotin/CTAAATTTTCTCTTGGAAACTCCCATTTGAGATCATA | 5′ biotin |
| NO. 23 | | TTCATATTCTCTGAAATCAACGTAGAAGTACTCATTATCTG | |
| | | AGGAGCCGGTCACCTGTACCATCTGTAGCTGGCTTTCATA | |
| | | CC | |
| SEQ ID | FLT3-7 | /biotin/TATTACTTGGGAGACTTGTCTGAACACTTCTTCCAG | 5′ biotin |
| NO. 24 | | GTCCAAGATGGTAATGGGTATCCATCCGAGAAACAGGACG | |
| | | CCTGACTTGCCGATGCTTCTGCGAGCACTTGAGGTTTCCCT | |
| | | ATA | |
| SEQ ID | FLT3-8 | /biotin/TGAACACTTCTTCCAGGTCCAAGATGGTAATGGGTA | 5′ biotin |
| NO. 25 | | TCCATCCGAGAAACAGGACGCCTGACTTGCCGATGCTTCT | |
| | | GCGAGCACTTGAGGTTTCCCTATAGAAAAGAACGTGTGAA | |
| | | ATAA | |
| SEQ ID | IDH2-1 | /biotin/CCCTCTCCACCCTGGCCTACCTGGTCGCCATGGGCG | 5′ biotin |
| NO. 26 | | TGCCTGCCAATGGTGATGGGCTTGGTCCAGCCAGGGACTA | |
| | | GGCGTGGGATGTTTTTGCAGATGATGGGCTCCCGGAAGAC | |
| | | AGTC | |
| SEQ ID | IDH2-2 | /biotin/TGCCAATGGTGATGGGCTTGGTCCAGCCAGGGACTA | 5′ biotin |
| NO. 27 | | GGCGTGGGATGTTTTTGCAGATGATGGGCTCCCGGAAGAC | |
| | | AGTCCCCCCCAGGATGTTCCGGATAGTTCCATTGGGACTTT | |
| | | TCC | |
| SEQ ID | TP53-1 | /biotin/CACTCACCTGGAGTGAGCCCTGCTCCCCCCTGGCTC | 5′ biotin |
| NO. 28 | | CTTCCCAGCCTGGGCATCCTTGAGTTCCAAGGCCTCATTCA | |
| | | GCTCTCGGAACATCTCGAAGCGCTCACGCCCACGGATCTG | |
| | | CAG | |
| SEQ ID | TP53-2 | /biotin/TGCTCCCCCCTGGCTCCTTCCCAGCCTGGGCATCCTT | 5′ biotin |
| NO. 29 | | GAGTTCCAAGGCCTCATTCAGCTCTCGGAACATCTCGAAG | |
| | | CGCTCACGCCCACGGATCTGCAGCAACAGAGGAGGGGGA | |
| | | GAAG | |
| SEQ ID | TP53-3 | /biotin/GTGCTCCCTGGGGGCAGCTCGTGGTGAGGCTCCCCT | 5′ biotin |
| NO. 30 | | TTCTTGCGGAGATTCTCTTCCTCTGTGCGCCGGTCTCTCCC | |
| | | AGGACAGGCACAAACACGCACCTCAAAGCTGTTCCGTCCC | |
| | | AGT | |
| SEQ ID | TP53-4 | /biotin/GTGGTGAGGCTCCCCTTTCTTGCGGAGATTCTCTTCC | 5′ biotin |
| NO. 31 | | TCTGTGCGCCGGTCTCTCCCAGGACAGGCACAAACACGCA | |
| | | CCTCAAAGCTGTTCCGTCCCAGTAGATTACCACTACTCAG | |
| | | GAT | |
| SEQ ID | TP53-5 | /biotin/CTGACCTGGAGTCTTCCAGTGTGATGATGGTGAGGA | 5′ biotin |
| NO. 32 | | TGGGCCTCCGGTTCATGCCGCCCATGCAGGAACTGTTACA | |
| | | CATGTAGTTGTAGTGGATGGTGGTACAGTCAGAGCCAACC | |
| | | TAGG | |
| SEQ ID | TP53-6 | /biotin/GTGATGATGGTGAGGATGGGCCTCCGGTTCATGCCG | 5′ biotin |
| NO. 33 | | CCCATGCAGGAACTGTTACACATGTAGTTGTAGTGGATGG | |
| | | TGGTACAGTCAGAGCCAACCTAGGAGATAACACAGGCCC | |
| | | AAGAT | |
| SEQ ID | TP53-7 | /biotin/AGACCTCAGGCGGCTCATAGGGCACCACCACACTAT | 5′ biotin |
| NO. 34 | | GTCGAAAAGTGTTTCTGTCATCCAAATACTCCACACGCAA | |
| | | ATTTCCTTCCACTCGGATAAGATGCTGAGGAGGGGCCAGA | |
| | | CCTA | |
| SEQ ID | TP53-8 | /biotin/GGCACCACCACACTATGTCGAAAAGTGTTTCTGTCA | 5′ biotin |
| NO. 35 | | TCCAAATACTCCACACGCAAATTTCCTTCCACTCGGATAA | |
| | | GATGCTGAGGAGGGGCCAGACCTAAGAGCAATCAGTGAG | |
| | | GAATC | |
| SEQ ID | TP53-9 | /biotin/CTCCAGCCCCAGCTGCTCACCATCGCTATCTGAGCA | 5′ biotin |
| NO. 36 | | GCGCTCATGGTGGGGGCAGCGCCTCACAACCTCCGTCATG | |
| | | TGCTGTGACTGCTTGTAGATGGCCATGGCGCGGACGCGGG | |
| | | TGCC | |
| SEQ ID | TP53-10 | /biotin/GCCTCACAACCTCCGTCATGTGCTGTGACTGCTTGT | 5′ biotin |
| NO. 37 | | AGATGGCCATGGCGCGGACGCGGGTGCCGGGGGGGGTG | |
| | | TGGAATCAACCCACAGCTGCACAGGGCAGGTCTTGGCCAG | |
| | | TTGGC | |
| SEQ ID | TP53-11 | /biotin/CGCGGACGCGGGTGCCGGGCGGGGGTGTGGAATCA | 5′ biotin |
| NO. 38 | | ACCCACAGCTGCACAGGGCAGGTCTTGGCCAGTTGGCAAA | |
| | | ACATCTTGTTGAGGGCAGGGGAGTACTGTAGGAAGAGGA | |
| | | AGGAGA | |
| SEQ ID | TP53-12 | /biotin/AATGCAAGAAGCCCAGACGGAAACCGTAGCTGCCC | 5′ biotin |
| NO. 39 | | TGGTAGGTTTTCTGGGAAGGGACAGAAGATGACAGGGGC | |
| | | CAGGAGGGGGCTGGTGCAGGGGCCGCCGGTGTAGGAGCT | |
| | | GCTGGTG | |
| SEQ ID | TP53-13 | /biotin/GAAGGGACAGAAGATGACAGGGGCCAGGAGGGGG | 5′ biotin |
| NO. 40 | | CTGGTGCAGGGGCCGCCGGTGTAGGAGCTGCTGGTGCAGG | |
| | | GGCCACGGGGGGAGCAGCCTCTGGCATTCTGGGAGCTTCA | |
| | | TCTGGA | |
| SEQ ID | TP53-14 | /biotin/GCCCTTCCAATGGATCCACTCACAGTTTCCATAGGT | 5′ biotin |
| NO. 41 | | CTGAAAATGTTTCCTGACTCAGAGGGGGCTCGACGCTAGG | |
| | | ATCTGACTGCGGCTCCTCCATGGCAGTGACCCGGAAGGCA | |
| | | GTCT | |
| SEQ ID | TP53-15 | /biotin/CACAGTTTCCATAGGTCTGAAAATGTTTCCTGACTC | 5′ biotin |
| NO. 42 | | AGAGGGGGCTCGACGCTAGGATCTGACTGCGGCTCCTCCA | |
| | | TGGCAGTGACCCGGAAGGCAGTCTGGCTGCTGCAAGAGG | |
| | | AAAAG | |
| SEQ ID | IDH1-1 | /biotin/TTATTGCCAACATGACTTACTTGATCCCCATAAGCA | 5′ biotin |
| NO. 43 | | TGACGACCTATGATGATAGGTTTTACCCATCCACTCACAA | |
| | | GCCGGGGGATATTTTTGCAGATAATGGCTTCTCTGAAGAC | |
| | | CGTG | |
| SEQ ID | IDH1-2 | /biotin/AGGTTTTACCCATCCACTCACAAGCCGGGGGATATT | 5′ biotin |
| NO. 44 | | TTTGCAGATAATGGCTTCTCTGAAGACCGTGCCACCCAGA | |
| | | ATATTTCGTATGGTGCCATTTGGTGATTTCCACATTTGTTTC | |
| | | AA | |

The most concentrated length distribution of plasma free DNA fragments is about 160 bp, so there may be not a probe capable of binding to a plasma free DNA fragment completely, and the overall binding of the probes to the target sequence is not stable. Furthermore, the proportion of the target region to the whole genome is very small, only about 1/2500000, and a low on-target rate result can also be expected.

To increase the probability of binding each fragment to probes, short probes are employed for capture. With a shorter probe length, it is hoped that there are 4 probes to which each fragment to be enriched of 160 bp can bind, i.e. the probe length does not exceed 40 nt. The target annealing temperature of each probe is set at 65° C. The annealing temperature is greatly influenced by a sequence base composition if the probe length is shorter, so the design method for the pool of probes is different from that of the conventional probes of 120 nt, and it is necessary to adjust the probe length within a certain range to make its annealing temperature close to a target value. Design of the pool of probes is performed according to part of the steps of the design method for the pool of probes provided by the present disclosure (a, d, g, preferably m1=40, and m2=5), the sum sequence is the human reference genome hg19, the target sequence is a target region sequence as shown in Table 3, and a probe length range parameter of 35-40 nt, and the probe annealing temperature of 65° C. are input. The resulting short probes are shown in Table 5, approximately 40 bp in length, with a total of 97 probes. After capture library NGS data analysis, it is shown that 99.9% of the sequences can be mapped to the human reference genome on average, wherein 23.4% of the sequences is located in the target region on average. Although there is a significant increase in the on-target rate, the on-target rate is still less than the requirement of conventional hybrid capture on the on-target rate of 50%. It is obvious that even if the probes are shortened directly, and the probe density is increased in overlapping probes for capture, the on-target rate cannot reach the basic requirement of 50%.

TABLE 5
Short probes covering the target region in Table 3

| | Sequence | | |
|---|---|---|---|
| | name | Sequence 5′-3′ | Modification |
| SEQ ID | NRAS-S-1 | /biotin/CAAATGCTGAAAGCTGTACCATACCTGTCTGGT | 5′ biotin |
| NO. 45 | | CT | |
| SEQ ID | NRAS-S-2 | /biotin/GCTGAGGTTTCAATGAATGGAATCCCGTAACTC | 5′ biotin |
| NO. 46 | | TT | |
| SEQ ID | NRAS-S-3 | /biotin/CCAGTTCGTGGGCTTGTTTTGTATCAACTGTCCT | 5′ biotin |
| NO. 47 | | T | |
| SEQ ID | NRAS-S-4 | /biotin/TGGCAAATCACACTTGTTTCCCACTAGCACCATA | 5′ biotin |
| NO. 48 | | G | |
| SEQ ID | NRAS-S-5 | /biotin/ACATCATCCGAGTCTTTTACTCGCTTAATCTGCT | 5′ biotin |
| NO. 49 | | C | |
| SEQ ID | NRAS-S-6 | /biotin/ACTTGCTATTATTGATGGCAAATACACAGAGGA | 5′ biotin |
| NO. 50 | | AGCC | |
| SEQ ID | NRAS-S-7 | /biotin/CGCCTGTCCTCATGTATTGGTCTCTCATGGCACT | 5′ biotin |
| NO. 51 | | G | |
| SEQ ID | NRAS-S-8 | /biotin/CTCTTCTTGTCCAGCTGTATCCAGTATGTCCAAC | 5′ biotin |
| NO. 52 | | A | |
| SEQ ID | NRAS-S-9 | /biotin/CAGGTTTCACCATCTATAACCACTTGTTTTCTGT | 5′ biotin |
| NO. 53 | | AAGAAT | |
| SEQ ID | NRAS-S-10 | /biotin/CCTGGGGGTGTGGAGGGTAAGGGGGCAGGGAG | 5′ biotin |
| NO. 54 | | GGA | |
| SEQ ID | NRAS-S-11 | /biotin/GGGCTACCACTGGGCCTCACCTCTATGGTGGGA | 5′ biotin |
| NO. 55 | | TC | |
| SEQ ID | NRAS-S-12 | /biotin/ATTCATCTACAAAGTGGTTCTGGATTAGCTGGAT | 5′ biotin |
| NO. 56 | | TGTC | |
| SEQ ID | NRAS-S-13 | /biotin/TGCGCTTTTCCCAACACCACCTGCTCCAACCACC | 5′ biotin |
| NO. 57 | | A | |
| SEQ ID | NRAS-S-14 | /biotin/AGTTTGTACTCAGTCATTTCACACCAGCAAGAA | 5′ biotin |
| NO. 58 | | CC | |
| SEQ ID | KRAS-S-1 | /biotin/TATTTATTTCAGTGTTACTTACCTGTCTTGTCTTT | 5′ biotin |
| NO. 59 | | GCTGA | |
| SEQ ID | KRAS-S-2 | /biotin/TGTTTCAATAAAAGGAATTCCATAACTTCTTGCT | 5′ biotin |
| NO. 60 | | AAGTCC | |
| SEQ ID | KRAS-S-3 | /biotin/TGAGCCTGTTTTGTGTCTACTGTTCTAGAAGGCA | 5′ biotin |
| NO. 61 | | A | |
| SEQ ID | KRAS-S-4 | /biotin/CACATTTATTTCCTACTAGGACCATAGGTACATC | 5′ biotin |
| NO. 62 | | TTCAG | |
| SEQ ID | KRAS-S-5 | /biotin/GTCCTTAACTCTTTTAATTTGTTCTCTGGGAAAG | 5′ biotin |
| NO. 63 | | AAAAAA | |
| SEQ ID | KRAS-S-6 | /biotin/AAGTTATAGCACAGTCATTAGTAACACAAATAT | 5′ biotin |
| NO. 64 | | CTTTCAA | |
| SEQ ID | KRAS-S-7 | /biotin/TAGTATTATTTATGGCAAATACACAAAGAAAGC | 5′ biotin |
| NO. 65 | | CCTCCCC | |
| SEQ ID | KRAS-S-8 | /biotin/AGTCCTCATGTACTGGTCCCTCATTGCACTGTAC | 5′ biotin |
| NO. 66 | | T | |
| SEQ ID | KRAS-S-9 | /biotin/TCTTGACCTGCTGTGTCGAGAATATCCAAGAGA | 5′ biotin |
| NO. 67 | | CA | |
| SEQ ID | KRAS-S-10 | /biotin/TTTCTCCATCAATTACTACTTGCTTCCTGTAGGA | 5′ biotin |
| NO. 68 | | ATCC | |
| SEQ ID | KRAS-S-11 | /biotin/AGAAGGGAGAAACACAGTCTGGATTATTACAGT | 5′ biotin |
| NO. 69 | | GC | |
| SEQ ID | KRAS-S-12 | /biotin/GATTTACCTCTATTGTTGGATCATATTCGTCCAC | 5′ biotin |
| NO. 70 | | AAAATG | |
| SEQ ID | KRAS-S-13 | /biotin/ATTCTGAATTAGCTGTATCGTCAAGGCACTCTTG | 5′ biotin |
| NO. 71 | | C | |
| SEQ ID | KRAS-S-14 | /biotin/ACGCCACCAGCTCCAACTACCACAAGTTTATAT | 5′ biotin |
| NO. 72 | | TC | |
| SEQ ID | KRAS-S-15 | /biotin/TCATTTTCAGCAGGCCTTATAATAAAAATAATG | 5′ biotin |
| NO. 73 | | AAAATGT | |
| SEQ ID | PTPN11-S-1 | /biotin/CTTTCCAATGGACTATTTTAGAAGAAATGGAGC | 5′ biotin |
| NO. 74 | | TGTCAC | |
| SEQ ID | PTPN11-S-2 | /biotin/CACATCAAGATTCAGAACACTGGTGATTACTAT | 5′ biotin |
| NO. 75 | | GACC | |
| SEQ ID | PTPN11-S-3 | /biotin/TATGGAGGGGAGAAATTTGCCACTTTGGCTGAG | 5′ biotin |
| NO. 76 | | TT | |
| SEQ ID | PTPN11-S-4 | /biotin/TCCAGTATTACATGGAACATCACGGGCAATTAA | 5′ biotin |
| NO. 77 | | AAGAG | |
| SEQ ID | PTPN11-S-5 | /biotin/GAATGGAGATGTCATTGAGCTTAAATATCCTCT | 5′ biotin |
| NO. 78 | | GAACTG | |
| SEQ ID | PTPN11-S-6 | /biotin/CTTCATGATGTTTCCTTCGTAGGTGTTGACTGCG | 5′ biotin |
| NO. 79 | | A | |
| SEQ ID | PTPN11-S-7 | /biotin/TTGACGTTCCCAAAACCATCCAGATGGTGCGGT | 5′ biotin |
| NO. 80 | | CT | |
| SEQ ID | PTPN11-S-8 | /biotin/GTACCGATTTATCTATATGGCGGTCCAGCATTAT | 5′ biotin |
| NO. 81 | | ATTG | |
| SEQ ID | PTPN11-S-9 | /biotin/ACACTACAGCGCAGGATTGAAGAAGAGCAGGT | 5′ biotin |
| NO. 82 | | ACC | |
| SEQ ID | PTPN11-S-10 | /biotin/CCTGAGGGCTGGCATGCGGATTCTCATTCTCTTG | 5′ biotin |
| NO. 83 | | C | |
| SEQ ID | FLT3-S-1 | /biotin/TAAGTAGGAAATAGCAGCCTCACATTGCCCCTG | 5′ biotin |
| NO. 84 | | AC | |
| SEQ ID | FLT3-S-2 | /biotin/CATAGTTGGAATCACTCATGATATCTCGAGCCA | 5′ biotin |
| NO. 85 | | ATC | |
| SEQ ID | FLT3-S-3 | /biotin/AAGTCACATATCTTCACCACTTTCCCGTGGGTGA | 5′ biotin |
| NO. 86 | | C | |
| SEQ ID | FLT3-S-4 | /biotin/GCACGTTCCTGGCGGCCAGGTCTCTGTGAACAC | 5′ biotin |
| NO. 87 | | AC | |
| SEQ ID | FLT3-S-5 | /biotin/GTGGGTTACCTGACAGTGTGCACGCCCCCAGCA | 5′ biotin |
| NO. 88 | | GG | |
| SEQ ID | FLT3-S-6 | /biotin/CACAATATTCTCGTGGCTTCCCAGCTGGGTCATC | 5′ biotin |
| NO. 89 | | A | |
| SEQ ID | FLT3-S-7 | /biotin/TTGAGTTCTGACATGAGTGCCTCTCTTTCAGAGC | 5′ biotin |
| NO. 90 | | T | |
| SEQ ID | FLT3-S-8 | /biotin/CTGCTTTTTCTGTCAAAGAAAGGAGCATTAAAA | 5′ biotin |
| NO. 91 | | ATGTAAA | |
| SEQ ID | FLT3-S-9 | /biotin/GGCACATTCCATTCTTACCAAACTCTAAATTTTC | 5′ biotin |
| NO. 92 | | TCTTGG | |
| SEQ ID | FLT3-S-10 | /biotin/AAACTCCCATTTGAGATCATATTCATATTCTCTG | 5′ biotin |
| NO. 93 | | AAATCA | |
| SEQ ID | FLT3-S-11 | /biotin/ACGTAGAAGTACTCATTATCTGAGGAGCCGGTC | 5′ biotin |
| NO. 94 | | AC | |
| SEQ ID | FLT3-S-12 | /biotin/GTACCATCTGTAGCTGGCTTTCATACCTAAATTG | 5′ biotin |
| NO. 95 | | C | |
| SEQ ID | FLT3-S-13 | /biotin/TATTACTTGGGAGACTTGTCTGAACACTTCTTCC | 5′ biotin |
| NO. 96 | | AG | |
| SEQ ID | FLT3-S-14 | /biotin/CCAAGATGGTAATGGGTATCCATCCGAGAAACA | 5′ biotin |
| NO. 97 | | GG | |
| SEQ ID | FLT3-S-15 | /biotin/GCCTGACTTGCCGATGCTTCTGCGAGCACTTGA | 5′ biotin |
| NO. 98 | | GG | |
| SEQ ID | FLT3-S-16 | /biotin/TCCCTATAGAAAAGAACGTGTGAAATAAGCTCA | 5′ biotin |
| NO. 99 | | CTGG | |
| SEQ ID | IDH2-S-1 | /biotin/ATCCCCTCTCCACCCTGGCCTACCTGGTCGCCAT | 5′ biotin |
| NO. 100 | | G | |
| SEQ ID | IDH2-S-2 | /biotin/CGTGCCTGCCAATGGTGATGGGCTTGGTCCAGC | 5′ biotin |
| NO. 101 | | CA | |
| SEQ ID | IDH2-S-3 | /biotin/GACTAGGCGTGGGATGTTTTTGCAGATGATGGG | 5′ biotin |
| NO. 102 | | CT | |
| SEQ ID | IDH2-S-4 | /biotin/CGGAAGACAGTCCCCCCCAGGATGTTCCGGATA | 5′ biotin |
| NO. 103 | | GT | |
| SEQ ID | IDH2-S-5 | /biotin/CATTGGGACTTTTCCACATCTTCTTCAGCTTGAA | 5′ biotin |
| NO. 104 | | C | |
| SEQ ID | TP53-S-1 | /biotin/AGGTCACTCACCTGGAGTGAGCCCTGCTCCCCC | 5′ biotin |
| NO. 105 | | CT | |
| SEQ ID | TP53-S-2 | /biotin/CTCCTTCCCAGCCTGGGCATCCTTGAGTTCCAAG | 5′ biotin |
| NO. 106 | | G | |
| SEQ ID | TP53-S-3 | /biotin/TCATTCAGCTCTCGGAACATCTCGAAGCGCTCA | 5′ biotin |
| NO. 107 | | CG | |
| SEQ ID | TP53-S-4 | /biotin/CACGGATCTGCAGCAACAGAGGAGGGGGAGAA | 5′ biotin |
| NO. 108 | | GTA | |
| SEQ ID | TP53-S-5 | /biotin/AGTGCTCCCTGGGGGCAGCTCGTGGTGAGGCTC | 5′ biotin |
| NO. 109 | | CC | |
| SEQ ID | TP53-S-6 | /biotin/TTCTTGCGGAGATTCTCTTCCTCTGTGCGCCGGT | 5′ biotin |
| NO. 110 | | C | |
| SEQ ID | TP53-S-7 | /biotin/TCCCAGGACAGGCACAAACACGCACCTCAAAGC | 5′ biotin |
| NO. 111 | | TG | |
| SEQ ID | TP53-S-8 | /biotin/CCGTCCCAGTAGATTACCACTACTCAGGATAGG | 5′ biotin |
| NO. 112 | | AA | |
| SEQ ID | TP53-S-9 | /biotin/CTCCTGACCTGGAGTCTTCCAGTGTGATGATGGT | 5′ biotin |
| NO. 113 | | G | |
| SEQ ID | TP53-S-10 | /biotin/GATGGGCCTCCGGTTCATGCCGCCCATGCAGGA | 5′ biotin |
| NO. 114 | | AC | |
| SEQ ID | TP53-S-11 | /biotin/TTACACATGTAGTTGTAGTGGATGGTGGTACAG | 5′ biotin |
| NO. 115 | | TC | |
| SEQ ID | TP53-S-12 | /biotin/AGCCAACCTAGGAGATAACACAGGCCCAAGAT | 5′ biotin |
| NO. 116 | | GAG | |
| SEQ ID | TP53-S-13 | /biotin/CCAGACCTCAGGCGGCTCATAGGGCACCACCAC | 5′ biotin |
| NO. 117 | | AC | |
| SEQ ID | TP53-S-14 | /biotin/TGTCGAAAAGTGTTTCTGTCATCCAAATACTCCA | 5′ biotin |
| NO. 118 | | CAC | |
| SEQ ID | TP53-S-15 | /biotin/AAATTTCCTTCCACTCGGATAAGATGCTGAGGA | 5′ biotin |
| NO. 119 | | GG | |
| SEQ ID | TP53-S-16 | /biotin/CCAGACCTAAGAGCAATCAGTGAGGAATCAGA | 5′ biotin |
| NO. 120 | | GGC | |
| SEQ ID | TP53-S-17 | /biotin/CTCCAGCCCCAGCTGCTCACCATCGCTATCTGAG | 5′ biotin |
| NO. 121 | | C | |
| SEQ ID | TP53-S-18 | /biotin/CGCTCATGGTGGGGGCAGCGCCTCACAACCTCC | 5′ biotin |
| NO. 122 | | GT | |
| SEQ ID | TP53-S-19 | /biotin/TGTGCTGTGACTGCTTGTAGATGGCCATGGCGC | 5′ biotin |
| NO. 123 | | GG | |
| SEQ ID | TP53-S-20 | /biotin/GCGGGTGCCGGGCGGGGGTGTGGAATCAACCCA | 5′ biotin |
| NO. 124 | | CA | |
| SEQ ID | TP53-S-21 | /biotin/TGCACAGGGCAGGTCTTGGCCAGTTGGCAAAAC | 5′ biotin |
| NO. 125 | | AT | |
| SEQ ID | TP53-S-22 | /biotin/TGTTGAGGGCAGGGGAGTACTGTAGGAAGAGG | 5′ biotin |
| NO. 126 | | AAG | |
| SEQ ID | TP53-S-23 | /biotin/GACAGAGTTGAAAGTCAGGGCACAAGTGAACA | 5′ biotin |
| NO. 127 | | GAT | |
| SEQ ID | TP53-S-24 | /biotin/AATGCAAGAAGCCCAGACGGAAACCGTAGCTG | 5′ biotin |
| NO. 128 | | CCC | |
| SEQ ID | TP53-S-25 | /biotin/GTAGGTTTTCTGGGAAGGGACAGAAGATGACAG | 5′ biotin |
| NO. 129 | | GG | |
| SEQ ID | TP53-S-26 | /biotin/CAGGAGGGGGCTGGTGCAGGGGCCGCCGGTGT | 5′ biotin |
| NO. 130 | | AGG | |
| SEQ ID | TP53-S-27 | /biotin/CTGCTGGTGCAGGGGCCACGGGGGGAGCAGCCT | 5′ biotin |
| NO. 131 | | CT | |
| SEQ ID | TP53-S-28 | /biotin/CATTCTGGGAGCTTCATCTGGACCTGGGTCTTCA | 5′ biotin |
| NO. 132 | | G | |
| SEQ ID | TP53-S-29 | /biotin/GCCCTTCCAATGGATCCACTCACAGTTTCCATAG | 5′ biotin |
| NO. 133 | | G | |
| SEQ ID | TP53-S-30 | /biotin/TGAAAATGTTTCCTGACTCAGAGGGGGCTCGAC | 5′ biotin |
| NO. 134 | | GC | |
| SEQ ID | TP53-S-31 | /biotin/GGATCTGACTGCGGCTCCTCCATGGCAGTGACC | 5′ biotin |
| NO. 135 | | CG | |
| SEQ ID | TP53-S-32 | /biotin/AGGCAGTCTGGCTGCTGCAAGAGGAAAAGTGG | 5′ biotin |
| NO. 136 | | GGA | |
| SEQ ID | IDH1-S-1 | /biotin/CATTATTGCCAACATGACTTACTTGATCCCCATA | 5′ biotin |
| NO. 137 | | AGC | |
| SEQ ID | IDH1-S-2 | /biotin/GACGACCTATGATGATAGGTTTTACCCATCCACT | 5′ biotin |
| NO. 138 | | C | |
| SEQ ID | IDH1-S-3 | /biotin/AAGCCGGGGGATATTTTTGCAGATAATGGCTTC | 5′ biotin |
| NO. 139 | | TC | |
| SEQ ID | IDH1-S-4 | /biotin/AAGACCGTGCCACCCAGAATATTTCGTATGGTG | 5′ biotin |
| NO. 140 | | CC | |
| SEQ ID | IDH1-S-5 | /biotin/TTGGTGATTTCCACATTTGTTTCAACTTGAACTC | 5′ biotin |
| NO. 141 | | CTCAAC | |

### Example 2: Comparison of a Hybrid Capture Effect of Conventional Probes of 120 bp With That of the NC Probes

In this Example, comparison of the capture results of a human plasma free DNA library by the NC probes and conventional probes of 120 bp with the capture results of the same target region in Example 1 is shown.

The NC probes are probes in which sequences for probes binding to each other are added to the short probe sequences shown in Table 5. According to the design method for the pool of probes provided by the present disclosure, the sum sequence is the human reference genome hg19, the target sequences are the target region sequence as shown in Table 3, the probe length range is set as 35-40 nt, and the probe annealing temperature is set as 65° C. The sequence length of a region wherein probes bind to each other is set as 8, i.e., k=8. A total of 65536 of all possible sequence combinations of 8 bases occur in the human reference genome hg19, with an average number of occurrences of 88419. From sequences with the lower number of occurrences, the selected probe binding sequence is CGTCGGTC, and its complementary sequence is GACCGACG, with a number of occurrences of 2078. This sequence is added to both sides of the probes in Table 5 as the probe binding sequence.

TABLE 6
NC probes covering the target region in Table 3

| | Sequence | | |
|---|---|---|---|
| | name | Sequence 5′-3′ | Modification |
| SEQ ID | NRAS-NC-1 | /biotin/CGTCGGTCCAAATGCTGAAAGCTGTACCATACCTG | 5′ biotin |
| NO. 142 | | TCTGGTCTGACCGACG | |
| SEQ ID | NRAS-NC-2 | /biotin/CGTCGGTCGCTGAGGTTTCAATGAATGGAATCCCG | 5′ biotin |
| NO. 143 | | TAACTCTTGACCGACG | |
| SEQ ID | NRAS-NC-3 | /biotin/CGTCGGTCCCAGTTCGTGGGCTTGTTTTGTATCAA | 5′ biotin |
| NO. 144 | | CTGTCCTTGACCGACG | |
| SEQ ID | NRAS-NC-4 | /biotin/CGTCGGTCTGGCAAATCACACTTGTTTCCCACTAG | 5′ biotin |
| NO. 145 | | CACCATAGGACCGACG | |
| SEQ ID | NRAS-NC-5 | /biotin/CGTCGGTCACATCATCCGAGTCTTTTACTCGCTTA | 5′ biotin |
| NO. 146 | | ATCTGCTCGACCGACG | |
| SEQ ID | NRAS-NC-6 | /biotin/CGTCGGTCACTTGCTATTATTGATGGCAAATACAC | 5′ biotin |
| NO. 147 | | AGAGGAAGCCGACCGACG | |
| SEQ ID | NRAS-NC-7 | /biotin/CGTCGGTCCGCCTGTCCTCATGTATTGGTCTCTCAT | 5′ biotin |
| NO. 148 | | GGCACTGGACCGACG | |
| SEQ ID | NRAS-NC-8 | /biotin/CGTCGGTCCTCTTCTTGTCCAGCTGTATCCAGTAT | 5′ biotin |
| NO. 149 | | GTCCAACAGACCGACG | |
| SEQ ID | NRAS-NC-9 | /biotin/CGTCGGTCCAGGTTTCACCATCTATAACCACTTGT | 5′ biotin |
| NO. 150 | | TTTCTGTAAGAATGACCGACG | |
| SEQ ID | NRAS-NC-10 | /biotin/CGTCGGTCCCTGGGGGTGTGGAGGGTAAGGGGGC | 5′ biotin |
| NO. 151 | | AGGGAGGGAGACCGACG | |
| SEQ ID | NRAS-NC-11 | /biotin/CGTCGGTCGGGCTACCACTGGGCCTCACCTCTATG | 5′ biotin |
| NO. 152 | | GTGGGATCGACCGACG | |
| SEQ ID | NRAS-NC-12 | /biotin/CGTCGGTCATTCATCTACAAAGTGGTTCTGGATTA | 5′ biotin |
| NO. 153 | | GCTGGATTGTCGACCGACG | |
| SEQ ID | NRAS-NC-13 | /biotin/CGTCGGTCTGCGCTTTTCCCAACACCACCTGCTCC | 5′ biotin |
| NO. 154 | | AACCACCAGACCGACG | |
| SEQ ID | NRAS-NC-14 | /biotin/CGTCGGTCAGTTTGTACTCAGTCATTTCACACCAG | 5′ biotin |
| NO. 155 | | CAAGAACCGACCGACG | |
| SEQ ID | KRAS-NC-1 | /biotin/CGTCGGTCTATTTATTTCAGTGTTACTTACCTGTCT | 5′ biotin |
| NO. 156 | | TGTCTTTGCTGAGACCGACG | |
| SEQ ID | KRAS-NC-2 | /biotin/CGTCGGTCTGTTTCAATAAAAGGAATTCCATAACT | 5′ biotin |
| NO. 157 | | TCTTGCTAAGTCCGACCGACG | |
| SEQ ID | KRAS-NC-3 | /biotin/CGTCGGTCTGAGCCTGTTTTGTGTCTACTGTTCTAG | 5′ biotin |
| NO. 158 | | AAGGCAAGACCGACG | |
| SEQ ID | KRAS-NC-4 | /biotin/CGTCGGTCCACATTTATTTCCTACTAGGACCATAG | 5′ biotin |
| NO. 159 | | GTACATCTTCAGGACCGACG | |
| SEQ ID | KRAS-NC-5 | /biotin/CGTCGGTCGTCCTTAACTCTTTTAATTTGTTCTCTG | 5′ biotin |
| NO. 160 | | GGAAAGAAAAAAGACCGACG | |
| SEQ ID | KRAS-NC-6 | /biotin/CGTCGGTCAAGTTATAGCACAGTCATTAGTAACAC | 5′ biotin |
| NO. 161 | | AAATATCTTTCAAGACCGACG | |
| SEQ ID | KRAS-NC-7 | /biotin/CGTCGGTCTAGTATTATTTATGGCAAATACACAAA | 5′ biotin |
| NO. 162 | | GAAAGCCCTCCCCGACCGACG | |
| SEQ ID | KRAS-NC-8 | /biotin/CGTCGGTCAGTCCTCATGTACTGGTCCCTCATTGC | 5′ biotin |
| NO. 163 | | ACTGTACTGACCGACG | |
| SEQ ID | KRAS-NC-9 | /biotin/CGTCGGTCTCTTGACCTGCTGTGTCGAGAATATCC | 5′ biotin |
| NO. 164 | | AAGAGACAGACCGACG | |
| SEQ ID | KRAS-NC-10 | /biotin/CGTCGGTCTTTCTCCATCAATTACTACTTGCTTCCT | 5′ biotin |
| NO. 165 | | GTAGGAATCCGACCGACG | |
| SEQ ID | KRAS-NC-11 | /biotin/CGTCGGTCAGAAGGGAGAAACACAGTCTGGATTA | 5′ biotin |
| NO. 166 | | TTACAGTGCGACCGACG | |
| SEQ ID | KRAS-NC-12 | /biotin/CGTCGGTCGATTTACCTCTATTGTTGGATCATATTC | 5′ biotin |
| NO. 167 | | GTCCACAAAATGGACCGACG | |
| SEQ ID | KRAS-NC-13 | /biotin/CGTCGGTCATTCTGAATTAGCTGTATCGTCAAGGC | 5′ biotin |
| NO. 168 | | ACTCTTGCGACCGACG | |
| SEQ ID | KRAS-NC-14 | /biotin/CGTCGGTCACGCCACCAGCTCCAACTACCACAAG | 5′ biotin |
| NO. 169 | | TTTATATTCGACCGACG | |
| SEQ ID | KRAS-NC-15 | /biotin/CGTCGGTCTCATTTTCAGCAGGCCTTATAATAAAA | 5′ biotin |
| NO. 170 | | ATAATGAAAATGTGACCGACG | |
| SEQ ID | PTPN11-NC-1 | /biotin/CGTCGGTCCTTTCCAATGGACTATTTTAGAAGAAA | 5′ biotin |
| NO. 171 | | TGGAGCTGTCACGACCGACG | |
| SEQ ID | PTPN11-NC-2 | /biotin/CGTCGGTCCACATCAAGATTCAGAACACTGGTGAT | 5′ biotin |
| NO. 172 | | TACTATGACCGACCGACG | |
| SEQ ID | PTPN11-NC-3 | /biotin/CGTCGGTCTATGGAGGGGAGAAATTTGCCACTTTG | 5′ biotin |
| NO. 173 | | GCTGAGTTGACCGACG | |
| SEQ ID | PTPN11-NC-4 | /biotin/CGTCGGTCTCCAGTATTACATGGAACATCACGGGC | 5′ biotin |
| NO. 174 | | AATTAAAAGAGGACCGACG | |
| SEQ ID | PTPN11-NC-5 | /biotin/CGTCGGTCGAATGGAGATGTCATTGAGCTTAAATA | 5′ biotin |
| NO. 175 | | TCCTCTGAACTGGACCGACG | |
| SEQ ID | PTPN11-NC-6 | /biotin/CGTCGGTCCTTCATGATGTTTCCTTCGTAGGTGTTG | 5′ biotin |
| NO. 176 | | ACTGCGAGACCGACG | |
| SEQ ID | PTPN11-NC-7 | /biotin/CGTCGGTCTTGACGTTCCCAAAACCATCCAGATGG | 5′ biotin |
| NO. 177 | | TGCGGTCTGACCGACG | |
| SEQ ID | PTPN11-NC-8 | /biotin/CGTCGGTCGTACCGATTTATCTATATGGCGGTCCA | 5′ biotin |
| NO. 178 | | GCATTATATTGGACCGACG | |
| SEQ ID | PTPN11-NC-9 | /biotin/CGTCGGTCACACTACAGCGCAGGATTGAAGAAGA | 5′ biotin |
| NO. 179 | | GCAGGTACCGACCGACG | |
| SEQ ID | PTPN11-NC-10 | /biotin/CGTCGGTCCCTGAGGGCTGGCATGCGGATTCTCAT | 5′ biotin |
| NO. 180 | | TCTCTTGCGACCGACG | |
| SEQ ID | FLT3-NC-1 | /biotin/CGTCGGTCTAAGTAGGAAATAGCAGCCTCACATT | 5′ biotin |
| NO. 181 | | GCCCCTGACGACCGACG | |
| SEQ ID | FLT3-NC-2 | /biotin/CGTCGGTCCATAGTTGGAATCACTCATGATATCTC | 5′ biotin |
| NO. 182 | | GAGCCAATCGACCGACG | |
| SEQ ID | FLT3-NC-3 | /biotin/CGTCGGTCAAGTCACATATCTTCACCACTTTCCCG | 5′ biotin |
| NO. 183 | | TGGGTGACGACCGACG | |
| SEQ ID | FLT3-NC-4 | /biotin/CGTCGGTCGCACGTTCCTGGCGGCCAGGTCTCTGT | 5′ biotin |
| NO. 184 | | GAACACACGACCGACG | |
| SEQ ID | FLT3-NC-5 | /biotin/CGTCGGTCGTGGGTTACCTGACAGTGTGCACGCCC | 5′ biotin |
| NO. 185 | | CCAGCAGGGACCGACG | |
| SEQ ID | FLT3-NC-6 | /biotin/CGTCGGTCCACAATATTCTCGTGGCTTCCCAGCTG | 5′ biotin |
| NO. 186 | | GGTCATCAGACCGACG | |
| SEQ ID | FLT3-NC-7 | /biotin/CGTCGGTCTTGAGTTCTGACATGAGTGCCTCTCTT | 5′ biotin |
| NO. 187 | | TCAGAGCTGACCGACG | |
| SEQ ID | FLT3-NC-8 | /biotin/CGTCGGTCCTGCTTTTTCTGTCAAAGAAAGGAGCA | 5′ biotin |
| NO. 188 | | TTAAAAATGTAAAGACCGACG | |
| SEQ ID | FLT3-NC-9 | /biotin/CGTCGGTCGGCACATTCCATTCTTACCAAACTCTA | 5′ biotin |
| NO. 189 | | AATTTTCTCTTGGGACCGACG | |
| SEQ ID | FLT3-NC-10 | /biotin/CGTCGGTCAAACTCCCATTTGAGATCATATTCATA | 5′ biotin |
| NO. 190 | | TTCTCTGAAATCAGACCGACG | |
| SEQ ID | FLT3-NC-11 | /biotin/CGTCGGTCACGTAGAAGTACTCATTATCTGAGGAG | 5′ biotin |
| NO. 191 | | CCGGTCACGACCGACG | |
| SEQ ID | FLT3-NC-12 | /biotin/CGTCGGTCGTACCATCTGTAGCTGGCTTTCATACC | 5′ biotin |
| NO. 192 | | TAAATTGCGACCGACG | |
| SEQ ID | FLT3-NC-13 | /biotin/CGTCGGTCTATTACTTGGGAGACTTGTCTGAACAC | 5′ biotin |
| NO. 193 | | TTCTTCCAGGACCGACG | |
| SEQ ID | FLT3-NC-14 | /biotin/CGTCGGTCCCAAGATGGTAATGGGTATCCATCCGA | 5′ biotin |
| NO. 194 | | GAAACAGGGACCGACG | |
| SEQ ID | FLT3-NC-15 | /biotin/CGTCGGTCGCCTGACTTGCCGATGCTTCTGCGAGC | 5′ biotin |
| NO. 195 | | ACTTGAGGGACCGACG | |
| SEQ ID | FLT3-NC-16 | /biotin/CGTCGGTCTCCCTATAGAAAAGAACGTGTGAAAT | 5′ biotin |
| NO. 196 | | AAGCTCACTGGGACCGACG | |
| SEQ ID | IDH2-NC-1 | /biotin/CGTCGGTCATCCCCTCTCCACCCTGGCCTACCTGG | 5′ biotin |
| NO. 197 | | TCGCCATGGACCGACG | |
| SEQ ID | IDH2-NC-2 | /biotin/CGTCGGTCCGTGCCTGCCAATGGTGATGGGCTTGG | 5′ biotin |
| NO. 198 | | TCCAGCCAGACCGACG | |
| SEQ ID | IDH2-NC-3 | /biotin/CGTCGGTCGACTAGGCGTGGGATGTTTTTGCAGAT | 5′ biotin |
| NO. 199 | | GATGGGCTGACCGACG | |
| SEQ ID | IDH2-NC-4 | /biotin/CGTCGGTCCGGAAGACAGTCCCCCCCAGGATGTTC | 5′ biotin |
| NO. 200 | | CGGATAGTGACCGACG | |
| SEQ ID | IDH2-NC-5 | /biotin/CGTCGGTCCATTGGGACTTTTCCACATCTTCTTCA | 5′ biotin |
| NO. 201 | | GCTTGAACGACCGACG | |
| SEQ ID | TP53-NC-1 | /biotin/CGTCGGTCAGGTCACTCACCTGGAGTGAGCCCTGC | 5′ biotin |
| NO. 202 | | TCCCCCCTGACCGACG | |
| SEQ ID | TP53-NC-2 | /biotin/CGTCGGTCCTCCTTCCCAGCCTGGGCATCCTTGAG | 5′ biotin |
| NO. 203 | | TTCCAAGGGACCGACG | |
| SEQ ID | TP53-NC-3 | /biotin/CGTCGGTCTCATTCAGCTCTCGGAACATCTCGAAG | 5′ biotin |
| NO. 204 | | CGCTCACGGACCGACG | |
| SEQ ID | TP53-NC-4 | /biotin/CGTCGGTCCACGGATCTGCAGCAACAGAGGAGGG | 5′ biotin |
| NO. 205 | | GGAGAAGTAGACCGACG | |
| SEQ ID | TP53-NC-5 | /biotin/CGTCGGTCAGTGCTCCCTGGGGGCAGCTCGTGGTG | 5′ biotin |
| NO. 206 | | AGGCTCCCGACCGACG | |
| SEQ ID | TP53-NC-6 | /biotin/CGTCGGTCTTCTTGCGGAGATTCTCTTCCTCTGTGC | 5′ biotin |
| NO. 207 | | GCCGGTCGACCGACG | |
| SEQ ID | TP53-NC-7 | /biotin/CGTCGGTCTCCCAGGACAGGCACAAACACGCACC | 5′ biotin |
| NO. 208 | | TCAAAGCTGGACCGACG | |
| SEQ ID | TP53-NC-8 | /biotin/CGTCGGTCCCGTCCCAGTAGATTACCACTACTCAG | 5′ biotin |
| NO. 209 | | GATAGGAAGACCGACG | |
| SEQ ID | TP53-NC-9 | /biotin/CGTCGGTCCTCCTGACCTGGAGTCTTCCAGTGTGA | 5′ biotin |
| NO. 210 | | TGATGGTGGACCGACG | |
| SEQ ID | TP53-NC-10 | /biotin/CGTCGGTCGATGGGCCTCCGGTTCATGCCGCCCAT | 5′ biotin |
| NO. 211 | | GCAGGAACGACCGACG | |
| SEQ ID | TP53-NC-11 | /biotin/CGTCGGTCTTACACATGTAGTTGTAGTGGATGGTG | 5′ biotin |
| NO. 212 | | GTACAGTCGACCGACG | |
| SEQ ID | TP53-NC-12 | /biotin/CGTCGGTCAGCCAACCTAGGAGATAACACAGGCC | 5′ biotin |
| NO. 213 | | CAAGATGAGGACCGACG | |
| SEQ ID | TP53-NC-13 | /biotin/CGTCGGTCCCAGACCTCAGGCGGCTCATAGGGCA | 5′ biotin |
| NO. 214 | | CCACCACACGACCGACG | |
| SEQ ID | TP53-NC-14 | /biotin/CGTCGGTCTGTCGAAAAGTGTTTCTGTCATCCAAA | 5′ biotin |
| NO. 215 | | TACTCCACACGACCGACG | |
| SEQ ID | TP53-NC-15 | /biotin/CGTCGGTCAAATTTCCTTCCACTCGGATAAGATGC | 5′ biotin |
| NO. 216 | | TGAGGAGGGACCGACG | |
| SEQ ID | TP53-NC-16 | /biotin/CGTCGGTCCCAGACCTAAGAGCAATCAGTGAGGA | 5′ biotin |
| NO. 217 | | ATCAGAGGCGACCGACG | |
| SEQ ID | TP53-NC-17 | /biotin/CGTCGGTCCTCCAGCCCCAGCTGCTCACCATCGCT | 5′ biotin |
| NO. 218 | | ATCTGAGCGACCGACG | |
| SEQ ID | TP53-NC-18 | /biotin/CGTCGGTCCGCTCATGGTGGGGGCAGCGCCTCAC | 5′ biotin |
| NO. 219 | | AACCTCCGTGACCGACG | |
| SEQ ID | TP53-NC-19 | /biotin/CGTCGGTCTGTGCTGTGACTGCTTGTAGATGGCCA | 5′ biotin |
| NO. 220 | | TGGCGCGGGACCGACG | |
| SEQ ID | TP53-NC-20 | /biotin/CGTCGGTCGCGGGTGCCGGGCGGGGGTGTGGAAT | 5′ biotin |
| NO. 221 | | CAACCCACAGACCGACG | |
| SEQ ID | TP53-NC-21 | /biotin/CGTCGGTCTGCACAGGGCAGGTCTTGGCCAGTTGG | 5′ biotin |
| NO. 222 | | CAAAACATGACCGACG | |
| SEQ ID | TP53-NC-22 | /biotin/CGTCGGTCTGTTGAGGGCAGGGGAGTACTGTAGG | 5′ biotin |
| NO. 223 | | AAGAGGAAGGACCGACG | |
| SEQ ID | TP53-NC-23 | /biotin/CGTCGGTCGACAGAGTTGAAAGTCAGGGCACAAG | 5′ biotin |
| NO. 224 | | TGAACAGATGACCGACG | |
| SEQ ID | TP53-NC-24 | /biotin/CGTCGGTCAATGCAAGAAGCCCAGACGGAAACCG | 5′ biotin |
| NO. 225 | | TAGCTGCCCGACCGACG | |
| SEQ ID | TP53-NC-25 | /biotin/CGTCGGTCGTAGGTTTTCTGGGAAGGGACAGAAG | 5′ biotin |
| NO. 226 | | ATGACAGGGGACCGACG | |
| SEQ ID | TP53-NC-26 | /biotin/CGTCGGTCCAGGAGGGGGCTGGTGCAGGGGCCGC | 5′ biotin |
| NO. 227 | | CGGTGTAGGGACCGACG | |
| SEQ ID | TP53-NC-27 | /biotin/CGTCGGTCCTGCTGGTGCAGGGGCCACGGGGGGA | 5′ biotin |
| NO. 228 | | GCAGCCTCTGACCGACG | |
| SEQ ID | TP53-NC-28 | /biotin/CGTCGGTCCATTCTGGGAGCTTCATCTGGACCTGG | 5′ biotin |
| NO. 229 | | GTCTTCAGGACCGACG | |
| SEQ ID | TP53-NC-29 | /biotin/CGTCGGTCGCCCTTCCAATGGATCCACTCACAGTT | 5′ biotin |
| NO. 230 | | TCCATAGGGACCGACG | |
| SEQ ID | TP53-NC-30 | /biotin/CGTCGGTCTGAAAATGTTTCCTGACTCAGAGGGGG | 5′ biotin |
| NO. 231 | | CTCGACGCGACCGACG | |
| SEQ ID | TP53-NC-31 | /biotin/CGTCGGTCGGATCTGACTGCGGCTCCTCCATGGCA | 5′ biotin |
| NO. 232 | | GTGACCCGGACCGACG | |
| SEQ ID | TP53-NC-32 | /biotin/CGTCGGTCAGGCAGTCTGGCTGCTGCAAGAGGAA | 5′ biotin |
| NO. 233 | | AAGTGGGGAGACCGACG | |
| SEQ ID | IDHI-NC-1 | /biotin/CGTCGGTCCATTATTGCCAACATGACTTACTTGAT | 5′ biotin |
| NO. 234 | | CCCCATAAGCGACCGACG | |
| SEQ ID | IDH1-NC-2 | /biotin/CGTCGGTCGACGACCTATGATGATAGGTTTTACCC | 5′ biotin |
| NO. 235 | | ATCCACTCGACCGACG | |
| SEQ ID | IDH1-NC-3 | /biotin/CGTCGGTCAAGCCGGGGGATATTTTTGCAGATAAT | 5′ biotin |
| NO. 236 | | GGCTTCTCGACCGACG | |
| SEQ ID | IDH1-NC-4 | /biotin/CGTCGGTCAAGACCGTGCCACCCAGAATATTTCGT | 5′ biotin |
| NO. 237 | | ATGGTGCCGACCGACG | |
| SEQ ID | IDH1-NC-5 | /biotin/CGTCGGTCTTGGTGATTTCCACATTTGTTTCAACTT | 5′ biotin |
| NO. 238 | | GAACTCCTCAACGACCGACG | |

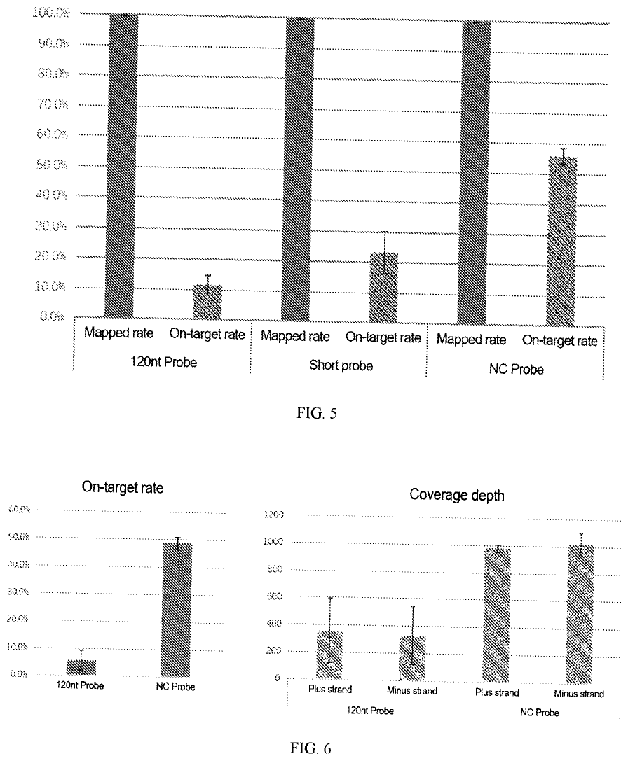

The results are shown in FIG. 5, NGS data of the NC probe captured library shows that 99.9% of the sequences can be mapped to the human reference genome, and an average proportion of sequences located in the target regions is 56.0%, which meets the on-target rate requirements in conventional hybrid capture.

### Example 3: Targeted Capture of a PCR-Free Library Using NC Probes

A PCR-free library refers to a library that is connected to a NGS adapter, but is not subjected to PCR amplification, wherein original sequence information is retained, and PCR preferences are not introduced. Hybrid capture with the PCR-free library directly suffers from the difficulties of low hybridization input and an unguaranteed capture rate. After PCR amplification of the library, each original fragment has multiple copies, so there are multiple opportunities to be bound and captured by probes. If any fragment in the PCR-free library is not captured by the probes, it cannot enter the next step, resulting in information loss. Moreover, after PCR, each single strand of the library fragment generates a corresponding complementary strand, so the probes only need to be designed in one direction to capture information from both strands of the original fragment. Whereas in the PCR-free library, both positive and negative strands of one fragment are present singly, and if the probes in only one direction are used for capture, the complementary chains will also be lost. Thus, in this Example, a probe of the other strand is added. The other strand probes for the conventional probes of 120 bp are shown in Table 7, and the other strand probes for the NC probes are shown in Table 8.

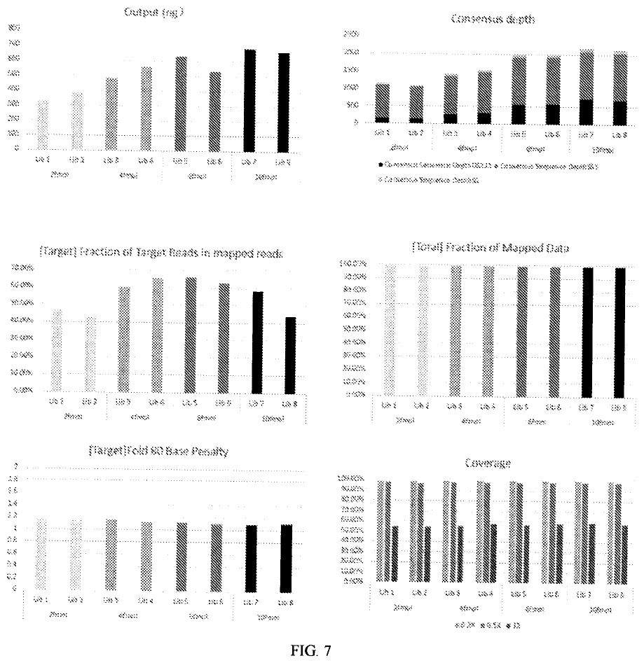

As shown in FIG. 6, after 30 ng of a plasma free DNA PCR-free library is captured by the conventional probes of 120 bp in Tables 4 and 7, the NGS results show an average on-target rate of only 5.6%, an average coverage depth of 356.1× of plus strands after deduplication, and an average depth of 329.9× of minus strands after deduplication. Whereas after capture by the NC probes shown in Tables 6 and 8, the NGS results show that the average on-target rate reaches 48.7%, with an average depth of 980.2× of the plus strands after deduplication, and an average depth of 1020.5× of the minus strands after deduplication. It can be seen that for the PCR-free library, the recovery rate and the on-target rate for the NC probes are greatly improved.

TABLE 7
Complementary strand probes for the conventional probes of 120 bp covering
the target region in Table 3

| | Sequence | | |
|---|---|---|---|
| | name | Sequence 5′-3′ | Modification |
| SEQ ID | NRAS-OP-1 | /biotin/GTGTGATTTGCCAACAAGGACAGTTGATACAAAAC | 5′ biotin |
| NO. 239 | | AAGCCCACGAACTGGCCAAGAGTTACGGGATTCCATTCA | |
| | | TTGAAACCTCAGCCAAGACCAGACAGGTATGGTACAGCT | |
| | | TTCAGCA | |
| SEQ ID | NRAS-OP-2 | /biotin/AAGACTCGGATGATGTACCTATGGTGCTAGTGGGA | 5′ biotin |
| NO. 240 | | AACAAGTGTGATTTGCCAACAAGGACAGTTGATACAAAA | |
| | | CAAGCCCACGAACTGGCCAAGAGTTACGGGATTCCATTC | |
| | | ATTGAAA | |
| SEQ ID | NRAS-OP-3 | /biotin/GGTGAAACCTGTTTGTTGGACATACTGGATACAGCT | 5′ biotin |
| NO. 241 | | GGACAAGAAGAGTACAGTGCCATGAGAGACCAATACATG | |
| | | AGGACAGGCGAAGGCTTCCTCTGTGTATTTGCCATCAATA | |
| | | ATAGC | |
| SEQ ID | NRAS-OP-4 | /biotin/CTTACCCTCCACACCCCCAGGATTCTTACAGAAAAC | 5′ biotin |
| NO. 242 | | AAGTGGTTATAGATGGTGAAACCTGTTTGTTGGACATACT | |
| | | GGATACAGCTGGACAAGAAGAGTACAGTGCCATGAGAGA | |
| | | CCAAT | |
| SEQ ID | NRAS-OP-5 | /biotin/AGTACAAACTGGTGGTGGTTGGAGCAGGTGGTGTT | 5′ biotin |
| NO. 243 | | GGGAAAAGCGCACTGACAATCCAGCTAATCCAGAACCAC | |
| | | TTTGTAGATGAATATGATCCCACCATAGAGGTGAGGCCCA | |
| | | GTGGTA | |
| SEQ ID | NRAS-OP-6 | /biotin/TGAAATGACTGAGTACAAACTGGTGGTGGTTGGAG | 5′ biotin |
| NO. 244 | | CAGGTGGTGTTGGGAAAAGCGCACTGACAATCCAGCTAA | |
| | | TCCAGAACCACTTTGTAGATGAATATGATCCCACCATAGA | |
| | | GGTGAG | |
| SEQ ID | KRAS-OP-1 | /biotin/ATGTGATTTGCCTTCTAGAACAGTAGACACAAAAC | 5′ biotin |
| NO. 245 | | AGGCTCAGGACTTAGCAAGAAGTTATGGAATTCCTTTTAT | |
| | | TGAAACATCAGCAAAGACAAGACAGGTAAGTAACACTGA | |
| | | AATAAA | |
| SEQ ID | KRAS-OP-2 | /biotin/AGGACTCTGAAGATGTACCTATGGTCCTAGTAGGA | 5′ biotin |
| NO. 246 | | AATAAATGTGATTTGCCTTCTAGAACAGTAGACACAAAA | |
| | | CAGGCTCAGGACTTAGCAAGAAGTTATGGAATTCCTTTTA | |
| | | TTGAAA | |
| SEQ ID | KRAS-OP-3 | /biotin/AACTTTTTTTCTTTCCCAGAGAACAAATTAAAAGAG | 5′ biotin |
| NO. 247 | | TTAAGGACTCTGAAGATGTACCTATGGTCCTAGTAGGAAA | |
| | | TAAATGTGATTTGCCTTCTAGAACAGTAGACACAAAACA | |
| | | GGCT | |
| SEQ ID | KRAS-OP-4 | /biotin/GGAGAAACCTGTCTCTTGGATATTCTCGACACAGC | 5′ biotin |
| NO. 248 | | AGGTCAAGAGGAGTACAGTGCAATGAGGGACCAGTACAT | |
| | | GAGGACTGGGGAGGGCTTTCTTTGTGTATTTGCCATAAAT | |
| | | AATACT | |
| SEQ ID | KRAS-OP-5 | /biotin/CTGTGTTTCTCCCTTCTCAGGATTCCTACAGGAAGC | 5′ biotin |
| NO. 249 | | AAGTAGTAATTGATGGAGAAACCTGTCTCTTGGATATTCT | |
| | | CGACACAGCAGGTCAAGAGGAGTACAGTGCAATGAGGGA | |
| | | CCAGT | |
| SEQ ID | KRAS-OP-6 | /biotin/GAAAATGACTGAATATAAACTTGTGGTAGTTGGAG | 5′ biotin |
| NO. 250 | | CTGGTGGCGTAGGCAAGAGTGCCTTGACGATACAGCTAA | |
| | | TTCAGAATCATTTTGTGGACGAATATGATCCAACAATAGA | |
| | | GGTAAA | |
| SEQ ID | KRAS-OP-7 | /biotin/TCATTATTTTTATTATAAGGCCTGCTGAAAATGACT | 5′ biotin |
| NO. 251 | | GAATATAAACTTGTGGTAGTTGGAGCTGGTGGCGTAGGC | |
| | | AAGAGTGCCTTGACGATACAGCTAATTCAGAATCATTTTG | |
| | | TGGAC | |
| SEQ ID | PTPN11-OP-1 | /biotin/CTGGACCAACTCAGCCAAAGTGGCAAATTTCTCCC | 5′ biotin |
| NO. 252 | | CTCCATACAGGTCATAGTAATCACCAGTGTTCTGAATCTT | |
| | | GATGTGGGTGACAGCTCCATTTCTTCTAAAATAGTCCATT | |
| | | GGAAA | |
| SEQ ID | PTPN11-OP-2 | /biotin/AAGCTCAATGACATCTCCATTCTTCTCTTTTAATTG | 5′ biotin |
| NO. 253 | | CCCGTGATGTTCCATGTAATACTGGACCAACTCAGCCAAA | |
| | | GTGGCAAATTTCTCCCCTCCATACAGGTCATAGTAATCAC | |
| | | CAGT | |
| SEQ ID | PTPN11-OP-3 | /biotin/ATATAGATAAATCGGTACTGTGCTTCTGTCTGGACC | 5′ biotin |
| NO. 254 | | ATCCCTGACCTCTGAGACCGCACCATCTGGATGGTTTTGG | |
| | | GAACGTCAATATCGCAGTCAACACCTACGAAGGAAACAT | |
| | | CATGA | |
| SEQ ID | PTPN11-OP-4 | /biotin/GCCAGCCCTCAGGCTGGTACCTGCTCTTCTTCAATC | 5′ biotin |
| NO. 255 | | CTGCGCTGTAGTGTTTCAATATAATGCTGGACCGCCATAT | |
| | | AGATAAATCGGTACTGTGCTTCTGTCTGGACCATCCCTGA | |
| | | CCTC | |
| SEQ ID | FLT3-OP-1 | /biotin/CCAGGAACGTGCTTGTCACCCACGGGAAAGTGGTG | 5′ biotin |
| | | AAGATATGTGACTTTGGATTGGCTCGAGATATCATGAGTG | |
| NO. 256 | | ATTCCAACTATGTTGTCAGGGGCAATGTGAGGCTGCTATT | |
| | | TCCTA | |
| SEQ ID | FLT3-OP-2 | /biotin/TGTTCACAGAGACCTGGCCGCCAGGAACGTGCTTG | 5′ biotin |
| NO. 257 | | TCACCCACGGGAAAGTGGTGAAGATATGTGACTTTGGATT | |
| | | GGCTCGAGATATCATGAGTGATTCCAACTATGTTGTCAGG | |
| | | GGCAA | |
| SEQ ID | FLT3-OP-3 | /biotin/ACAGAAAAAGCAGACAGCTCTGAAAGAGAGGCAC | 5′ biotin |
| NO. 258 | | TCATGTCAGAACTCAAGATGATGACCCAGCTGGGAAGCC | |
| | | ACGAGAATATTGTGAACCTGCTGGGGGCGTGCACACTGT | |
| | | CAGGTAAC | |
| SEQ ID | FLT3-OP-4 | /biotin/TTTTAATGCTCCTTTCTTTGACAGAAAAAGCAGACA | 5′ biotin |
| NO. 259 | | GCTCTGAAAGAGAGGCACTCATGTCAGAACTCAAGATGA | |
| | | TGACCCAGCTGGGAAGCCACGAGAATATTGTGAACCTGC | |
| | | TGGGGG | |
| SEQ ID | FLT3-OP-5 | /biotin/ATGGTACAGGTGACCGGCTCCTCAGATAATGAGTA | 5′ biotin |
| NO. 260 | | CTTCTACGTTGATTTCAGAGAATATGAATATGATCTCAAA | |
| | | TGGGAGTTTCCAAGAGAAAATTTAGAGTTTGGTAAGAAT | |
| | | GGAATG | |
| SEQ ID | FLT3-OP-6 | /biotin/GGTATGAAAGCCAGCTACAGATGGTACAGGTGACC | 5′ biotin |
| NO. 261 | | GGCTCCTCAGATAATGAGTACTICTACGTTGATTTCAGAG | |
| | | AATATGAATATGATCTCAAATGGGAGTTTCCAAGAGAAA | |
| | | ATTTAG | |
| SEQ ID | FLT3-OP-7 | /biotin/TATAGGGAAACCTCAAGTGCTCGCAGAAGCATCGG | 5′ biotin |
| NO. 262 | | CAAGTCAGGCGTCCTGTTTCTCGGATGGATACCCATTACC | |
| | | ATCTTGGACCTGGAAGAAGTGTTCAGACAAGTCTCCCAA | |
| | | GTAATA | |
| SEQ ID | FLT3-OP-8 | /biotin/TTATTTCACACGTTCTTTTCTATAGGGAAACCTCAA | 5′ biotin |
| NO. 263 | | GTGCTCGCAGAAGCATCGGCAAGTCAGGCGTCCTGTTTCT | |
| | | CGGATGGATACCCATTACCATCTTGGACCTGGAAGAAGT | |
| | | GTTCA | |
| SEQ ID | IDH2-OP-1 | /biotin/GACTGTCTTCCGGGAGCCCATCATCTGCAAAAACA | 5′ biotin |
| NO. 264 | | TCCCACGCCTAGTCCCTGGCTGGACCAAGCCCATCACCAT | |
| | | TGGCAGGCACGCCCATGGCGACCAGGTAGGCCAGGGTGG | |
| | | AGAGGG | |
| SEQ ID | IDH2-OP-2 | /biotin/GGAAAAGTCCCAATGGAACTATCCGGAACATCCTG | 5′ biotin |
| NO. 265 | | GGGGGGACTGTCTTCCGGGAGCCCATCATCTGCAAAAAC | |
| | | ATCCCACGCCTAGTCCCTGGCTGGACCAAGCCCATCACCA | |
| | | TTGGCA | |
| SEQ ID | TP53-OP-1 | /biotin/CTGCAGATCCGTGGGCGTGAGCGCTTCGAGATGTT | 5′ biotin |
| NO. 266 | | CCGAGAGCTGAATGAGGCCTTGGAACTCAAGGATGCCCA | |
| | | GGCTGGGAAGGAGCCAGGGGGGAGCAGGGCTCACTCCAG | |
| | | GTGAGTG | |
| SEQ ID | TP53-OP-2 | /biotin/CTTCTCCCCCTCCTCTGTTGCTGCAGATCCGTGGGC | 5′ biotin |
| NO. 267 | | GTGAGCGCTTCGAGATGTTCCGAGAGCTGAATGAGGCCTT | |
| | | GGAACTCAAGGATGCCCAGGCTGGGAAGGAGCCAGGGG | |
| | | GGAGCA | |
| SEQ ID | TP53-OP-3 | /biotin/ACTGGGACGGAACAGCTTTGAGGTGCGTGTTTGTG | 5′ biotin |
| NO. 268 | | CCTGTCCTGGGAGAGACCGGCGCACAGAGGAAGAGAATC | |
| | | TCCGCAAGAAAGGGGAGCCTCACCACGAGCTGCCCCCAG | |
| | | GGAGCAC | |
| SEQ ID | TP53-OP-4 | /biotin/ATCCTGAGTAGTGGTAATCTACTGGGACGGAACAG | 5′ biotin |
| NO. 269 | | CTTTGAGGTGCGTGTTTGTGCCTGTCCTGGGAGAGACCGG | |
| | | CGCACAGAGGAAGAGAATCTCCGCAAGAAAGGGGAGCCT | |
| | | CACCAC | |
| SEQ ID | TP53-OP-5 | /biotin/CCTAGGTTGGCTCTGACTGTACCACCATCCACTACA | 5′ biotin |
| NO. 270 | | ACTACATGTGTAACAGTTCCTGCATGGGCGGCATGAACCG | |
| | | GAGGCCCATCCTCACCATCATCACACTGGAAGACTCCAG | |
| | | GTCAG | |
| SEQ ID | TP53-OP-6 | /biotin/ATCTTGGGCCTGTGTTATCTCCTAGGTTGGCTCTGA | 5′ biotin |
| NO. 271 | | CTGTACCACCATCCACTACAACTACATGTGTAACAGTTCC | |
| | | TGCATGGGCGGCATGAACCGGAGGCCCATCCTCACCATC | |
| | | ATCAC | |
| SEQ ID | TP53-OP-7 | /biotin/TAGGTCTGGCCCCTCCTCAGCATCTTATCCGAGTGG | 5′ biotin |
| NO. 272 | | AAGGAAATTTGCGTGTGGAGTATTTGGATGACAGAAACA | |
| | | CTTTTCGACATAGTGTGGTGGTGCCCTATGAGCCGCCTGA | |
| | | GGTCT | |
| SEQ ID | TP53-OP-8 | /biotin/GATTCCTCACTGATTGCTCTTAGGTCTGGCCCCTCC | 5′ biotin |
| NO. 273 | | TCAGCATCTTATCCGAGTGGAAGGAAATTTGCGTGTGGAG | |
| | | TATTTGGATGACAGAAACACTTTTCGACATAGTGTGGTGG | |
| | | TGCC | |
| SEQ ID | TP53-OP-9 | /biotin/GGCACCCGCGTCCGCGCCATGGCCATCTACAAGCA | 5′ biotin |
| NO. 274 | | GTCACAGCACATGACGGAGGTTGTGAGGCGCTGCCCCCA | |
| | | CCATGAGCGCTGCTCAGATAGCGATGGTGAGCAGCTGGG | |
| | | GCTGGAG | |
| SEQ ID | TP53-OP-10 | /biotin/GCCAACTGGCCAAGACCTGCCCTGTGCAGCTGTGG | 5′ biotin |
| NO. 275 | | GTTGATTCCACACCCCCGCCCGGCACCCGCGTCCGCGCCA | |
| | | TGGCCATCTACAAGCAGTCACAGCACATGACGGAGGTTG | |
| | | TGAGGC | |
| SEQ ID | TP53-OP-11 | /biotin/TCTCCTTCCTCTTCCTACAGTACTCCCCTGCCCTCAA | 5′ biotin |
| NO. 276 | | CAAGATGTTTTGCCAACTGGCCAAGACCTGCCCTGTGCAG | |
| | | CTGTGGGTTGATTCCACACCCCCGCCCGGCACCCGCGTCC | |
| | | GCG | |
| SEQ ID | TP53-OP-12 | /biotin/CACCAGCAGCTCCTACACCGGCGGCCCCTGCACCA | 5′ biotin |
| NO. 277 | | GCCCCCTCCTGGCCCCTGTCATCTTCTGTCCCTTCCCAGAA | |
| | | AACCTACCAGGGCAGCTACGGTTTCCGTCTGGGCTTCTTG | |
| | | CATT | |
| SEQ ID | TP53-OP-13 | /biotin/TCCAGATGAAGCTCCCAGAATGCCAGAGGCTGCTC | 5′ biotin |
| NO. 278 | | CCCCCGTGGCCCCTGCACCAGCAGCTCCTACACCGGCGGC | |
| | | CCCTGCACCAGCCCCCTCCTGGCCCCTGTCATCTTCTGTCC | |
| | | CTTC | |
| SEQ ID | TP53-OP-14 | /biotin/AGACTGCCTTCCGGGTCACTGCCATGGAGGAGCCG | 5′ biotin |
| NO. 279 | | CAGTCAGATCCTAGCGTCGAGCCCCCTCTGAGTCAGGAA | |
| | | ACATTTTCAGACCTATGGAAACTGTGAGTGGATCCATTGG | |
| | | AAGGGC | |
| SEQ ID | TP53-OP-15 | /biotin/CTTTTCCTCTTGCAGCAGCCAGACTGCCTTCCGGGT | 5′ biotin |
| NO. 280 | | CACTGCCATGGAGGAGCCGCAGTCAGATCCTAGCGTCGA | |
| | | GCCCCCTCTGAGTCAGGAAACATTTTCAGACCTATGGAAA | |
| | | CTGTG | |
| SEQ ID | IDH1-OP-1 | /biotin/CACGGTCTTCAGAGAAGCCATTATCTGCAAAAATA | 5′ biotin |
| NO. 281 | | TCCCCCGGCTTGTGAGTGGATGGGTAAAACCTATCATCAT | |
| | | AGGTCGTCATGCTTATGGGGATCAAGTAAGTCATGTTGGC | |
| | | AATAA | |
| SEQ ID | IDH1-OP-2 | /biotin/TTGAAACAAATGTGGAAATCACCAAATGGCACCAT | 5′ biotin |
| NO. 282 | | ACGAAATATTCTGGGTGGCACGGTCTTCAGAGAAGCCATT | |
| | | ATCTGCAAAAATATCCCCCGGCTTGTGAGTGGATGGGTAA | |
| | | AACCT | |

TABLE 8
Complementary strand probes for the NC probes covering the target region in Table 3

| | Sequence | | |
|---|---|---|---|
| | name | Sequence 5′-3′ | Modification |
| SEQ ID | NRAS-NC-OP-1 | /biotin/CGTCGGTCAGACCAGACAGGTATGGTACAGCTTTC | 5′ biotin |
| NO. 283 | | AGCATTTGGACCGACG | |
| SEQ ID | NRAS-NC-OP-2 | /biotin/CGTCGGTCAAGAGTTACGGGATTCCATTCATTGAA | 5′ biotin |
| NO. 284 | | ACCTCAGCGACCGACG | |
| SEQ ID | NRAS-NC-OP-3 | /biotin/CGTCGGTCAAGGACAGTTGATACAAAACAAGCCC | 5′ biotin |
| NO. 285 | | ACGAACTGGGACCGACG | |
| SEQ ID | NRAS-NC-OP-4 | /biotin/CGTCGGTCCTATGGTGCTAGTGGGAAACAAGTGTG | 5′ biotin |
| NO. 286 | | ATTTGCCAGACCGACG | |
| SEQ ID | NRAS-NC-OP-5 | /biotin/CGTCGGTCGAGCAGATTAAGCGAGTAAAAGACTC | 5′ biotin |
| NO. 287 | | GGATGATGTGACCGACG | |
| SEQ ID | NRAS-NC-OP-6 | /biotin/CGTCGGTCGGCTTCCTCTGTGTATTTGCCATCAATA | 5′ biotin |
| NO. 288 | | ATAGCAAGTGACCGACG | |
| SEQ ID | NRAS-NC-OP-7 | /biotin/CGTCGGTCCAGTGCCATGAGAGACCAATACATGA | 5′ biotin |
| NO. 289 | | GGACAGGCGGACCGACG | |
| SEQ ID | NRAS-NC-OP-8 | /biotin/CGTCGGTCTGTTGGACATACTGGATACAGCTGGAC | 5′ biotin |
| NO. 290 | | AAGAAGAGGACCGACG | |
| SEQ ID | NRAS-NC-OP-9 | /biotin/CGTCGGTCATTCTTACAGAAAACAAGTGGTTATAG | 5′ biotin |
| NO. 291 | | ATGGTGAAACCTGGACCGACG | |
| SEQ ID | NRAS-NC-OP-10 | /biotin/CGTCGGTCTCCCTCCCTGCCCCCTTACCCTCCACAC | 5′ biotin |
| NO. 292 | | CCCCAGGGACCGACG | |
| SEQ ID | NRAS-NC-OP-11 | /biotin/CGTCGGTCGATCCCACCATAGAGGTGAGGCCCAGT | 5′ biotin |
| NO. 293 | | GGTAGCCCGACCGACG | |
| SEQ ID | NRAS-NC-OP-12 | /biotin/CGTCGGTCGACAATCCAGCTAATCCAGAACCACTT | 5′ biotin |
| NO. 294 | | TGTAGATGAATGACCGACG | |
| SEQ ID | NRAS-NC-OP-13 | /biotin/CGTCGGTCTGGTGGTTGGAGCAGGTGGTGTTGGGA | 5′ biotin |
| NO. 295 | | AAAGCGCAGACCGACG | |
| SEQ ID | NRAS-NC-OP-14 | /biotin/CGTCGGTCGGTTCTTGCTGGTGTGAAATGACTGAG | 5′ biotin |
| NO. 296 | | TACAAACTGACCGACG | |
| SEQ ID | KRAS-NC-OP-1 | /biotin/CGTCGGTCTCAGCAAAGACAAGACAGGTAAGTAA | 5′ biotin |
| NO. 297 | | CACTGAAATAAATAGACCGACG | |
| SEQ ID | KRAS-NC-OP-2 | /biotin/CGTCGGTCGGACTTAGCAAGAAGTTATGGAATTCC | 5′ biotin |
| NO. 298 | | TTTTATTGAAACAGACCGACG | |
| SEQ ID | KRAS-NC-OP-3 | /biotin/CGTCGGTCTTGCCTTCTAGAACAGTAGACACAAAA | 5′ biotin |
| NO. 299 | | CAGGCTCAGACCGACG | |
| SEQ ID | KRAS-NC-OP-4 | /biotin/CGTCGGTCCTGAAGATGTACCTATGGTCCTAGTAG | 5′ biotin |
| NO. 300 | | GAAATAAATGTGGACCGACG | |
| SEQ ID | KRAS-NC-OP-5 | /biotin/CGTCGGTCTTTTTTCTTTCCCAGAGAACAAATTAA | 5′ biotin |
| NO. 301 | | AAGAGTTAAGGACGACCGACG | |
| SEQ ID | KRAS-NC-OP-6 | /biotin/CGTCGGTCTTGAAAGATATTTGTGTTACTAATGAC | 5′ biotin |
| NO. 302 | | TGTGCTATAACTTGACCGACG | |
| SEQ ID | KRAS-NC-OP-7 | /biotin/CGTCGGTCGGGGAGGGCTTTCTTTGTGTATTTGCC | 5′ biotin |
| NO. 303 | | ATAAATAATACTAGACCGACG | |
| SEQ ID | KRAS-NC-OP-8 | /biotin/CGTCGGTCAGTACAGTGCAATGAGGGACCAGTAC | 5′ biotin |
| NO. 304 | | ATGAGGACTGACCGACG | |
| SEQ ID | KRAS-NC-OP-9 | /biotin/CGTCGGTCTGTCTCTTGGATATTCTCGACACAGCA | 5′ biotin |
| NO. 305 | | GGTCAAGAGACCGACG | |
| SEQ ID | KRAS-NC-OP-10 | /biotin/CGTCGGTCGGATTCCTACAGGAAGCAAGTAGTAAT | 5′ biotin |
| NO. 306 | | TGATGGAGAAAGACCGACG | |
| SEQ ID | KRAS-NC-OP-11 | /biotin/CGTCGGTCGCACTGTAATAATCCAGACTGTGTTTC | 5′ biotin |
| NO. 307 | | TCCCTTCTGACCGACG | |
| SEQ ID | KRAS-NC-OP-12 | /biotin/CGTCGGTCCATTTTGTGGACGAATATGATCCAACA | 5′ biotin |
| NO. 308 | | ATAGAGGTAAATCGACCGACG | |
| SEQ ID | KRAS-NC-OP-13 | /biotin/CGTCGGTCGCAAGAGTGCCTTGACGATACAGCTAA | 5′ biotin |
| NO. 309 | | TTCAGAATGACCGACG | |
| SEQ ID | KRAS-NC-OP-14 | /biotin/CGTCGGTCGAATATAAACTTGTGGTAGTTGGAGCT | 5′ biotin |
| NO. 310 | | GGTGGCGTGACCGACG | |
| SEQ ID | KRAS-NC-OP-15 | /biotin/CGTCGGTCACATTTTCATTATTTTTATTATAAGGCC | 5′ biotin |
| NO. 311 | | TGCTGAAAATGAGACCGACG | |
| SEQ ID | PTPN11-NC- | /biotin/CGTCGGTCGTGACAGCTCCATTTCTTCTAAAATAG | 5′ biotin |
| NO. 312 | OP-1 | TCCATTGGAAAGGACCGACG | |
| SEQ ID | PTPN11-NC- | /biotin/CGTCGGTCGGTCATAGTAATCACCAGTGTTCTGAA | 5′ biotin |
| NO. 313 | OP-2 | TCTTGATGTGGACCGACG | |
| SEQ ID | PTPN11-NC- | /biotin/CGTCGGTCAACTCAGCCAAAGTGGCAAATTTCTCC | 5′ biotin |
| NO. 314 | OP-3 | CCTCCATAGACCGACG | |
| SEQ ID | PTPN11-NC- | /biotin/CGTCGGTCCTCTTTTAATTGCCCGTGATGTTCCATG | 5′ biotin |
| NO. 315 | OP-4 | TAATACTGGAGACCGACG | |
| SEQ ID | PTPN11-NC- | /biotin/CGTCGGTCCAGTTCAGAGGATATTTAAGCTCAATG | 5′ biotin |
| NO. 316 | OP-5 | ACATCTCCATTCGACCGACG | |
| SEQ ID | PTPN11-NC- | /biotin/CGTCGGTCTCGCAGTCAACACCTACGAAGGAAAC | 5′ biotin |
| NO. 317 | OP-6 | ATCATGAAGGACCGACG | |
| SEQ ID | PTPN11-NC- | /biotin/CGTCGGTCAGACCGCACCATCTGGATGGTTTTGGG | 5′ biotin |
| NO. 318 | OP-7 | AACGTCAAGACCGACG | |
| SEQ ID | PTPN11-NC- | /biotin/CGTCGGTCCAATATAATGCTGGACCGCCATATAGA | 5′ biotin |
| NO. 319 | OP-8 | TAAATCGGTACGACCGACG | |
| SEQ ID | PTPN11-NC- | /biotin/CGTCGGTCGGTACCTGCTCTTCTTCAATCCTGCGCT | 5′ biotin |
| NO. 320 | OP-9 | GTAGTGTGACCGACG | |
| SEQ ID | PTPN11-NC- | /biotin/CGTCGGTCGCAAGAGAATGAGAATCCGCATGCCA | 5′ biotin |
| NO. 321 | OP-10 | GCCCTCAGGGACCGACG | |
| SEQ ID | FLT3-NC-OP- | /biotin/CGTCGGTCGTCAGGGGCAATGTGAGGCTGCTATTT | 5′ biotin |
| NO. 322 | 1 | CCTACTTAGACCGACG | |
| SEQ ID | FLT3-NC-OP- | /biotin/CGTCGGTCGATTGGCTCGAGATATCATGAGTGATT | 5′ biotin |
| NO. 323 | 2 | CCAACTATGGACCGACG | |
| SEQ ID | FLT3-NC-OP- | /biotin/CGTCGGTCGTCACCCACGGGAAAGTGGTGAAGAT | 5′ biotin |
| NO. 324 | 3 | ATGTGACTTGACCGACG | |
| SEQ ID | FLT3-NC-OP- | /biotin/CGTCGGTCGTGTGTTCACAGAGACCTGGCCGCCAG | 5′ biotin |
| NO. 325 | 4 | GAACGTGCGACCGACG | |
| SEQ ID | FLT3-NC-OP- | /biotin/CGTCGGTCCCTGCTGGGGGCGTGCACACTGTCAGG | 5′ biotin |
| NO. 326 | 5 | TAACCCACGACCGACG | |
| SEQ ID | FLT3-NC-OP- | /biotin/CGTCGGTCTGATGACCCAGCTGGGAAGCCACGAG | 5′ biotin |
| NO. 327 | 6 | AATATTGTGGACCGACG | |
| SEQ ID | FLT3-NC-OP- | /biotin/CGTCGGTCAGCTCTGAAAGAGAGGCACTCATGTCA | 5′ biotin |
| NO. 328 | 7 | GAACTCAAGACCGACG | |
| SEQ ID | FLT3-NC-OP- | /biotin/CGTCGGTCTTTACATTTTTAATGCTCCTTTCTTTGA | 5′ biotin |
| NO. 329 | 8 | CAGAAAAAGCAGGACCGACG | |
| SEQ ID | FLT3-NC-OP- | /biotin/CGTCGGTCCCAAGAGAAAATTTAGAGTTTGGTAAG | 5′ biotin |
| NO. 330 | 9 | AATGGAATGTGCCGACCGACG | |
| SEQ ID | FLT3-NC-OP- | /biotin/CGTCGGTCTGATTTCAGAGAATATGAATATGATCT | 5′ biotin |
| NO. 331 | 10 | CAAATGGGAGTTTGACCGACG | |
| SEQ ID | FLT3-NC-OP- | /biotin/CGTCGGTCGTGACCGGCTCCTCAGATAATGAGTAC | 5′ biotin |
| NO. 332 | 11 | TTCTACGTGACCGACG | |
| SEQ ID | FLT3-NC-OP- | /biotin/CGTCGGTCGCAATTTAGGTATGAAAGCCAGCTACA | 5′ biotin |
| NO. 333 | 12 | GATGGTACGACCGACG | |
| SEQ ID | FLT3-NC-OP- | /biotin/CGTCGGTCCTGGAAGAAGTGTTCAGACAAGTCTCC | 5′ biotin |
| NO. 334 | 13 | CAAGTAATAGACCGACG | |
| SEQ ID | FLT3-NC-OP- | /biotin/CGTCGGTCCCTGTTTCTCGGATGGATACCCATTAC | 5′ biotin |
| NO. 335 | 14 | CATCTTGGGACCGACG | |
| SEQ ID | FLT3-NC-OP- | /biotin/CGTCGGTCCCTCAAGTGCTCGCAGAAGCATCGGCA | 5′ biotin |
| NO. 336 | 15 | AGTCAGGCGACCGACG | |
| SEQ ID | FLT3-NC-OP- | /biotin/CGTCGGTCCCAGTGAGCTTATTTCACACGTTCTTTT | 5′ biotin |
| NO. 337 | 16 | CTATAGGGAGACCGACG | |
| SEQ ID | IDH2-NC-OP- | /biotin/CGTCGGTCCATGGCGACCAGGTAGGCCAGGGTGG | 5′ biotin |
| NO. 338 | 1 | AGAGGGGATGACCGACG | |
| SEQ ID | IDH2-NC-OP- | /biotin/CGTCGGTCTGGCTGGACCAAGCCCATCACCATTGG | 5′ biotin |
| NO. 339 | 2 | CAGGCACGGACCGACG | |
| SEQ ID | IDH2-NC-OP- | /biotin/CGTCGGTCAGCCCATCATCTGCAAAAACATCCCAC | 5′ biotin |
| NO. 340 | 3 | GCCTAGTCGACCGACG | |
| SEQ ID | IDH2-NC-OP- | /biotin/CGTCGGTCACTATCCGGAACATCCTGGGGGGGACT | 5′ biotin |
| NO. 341 | 4 | GTCTTCCGGACCGACG | |
| SEQ ID | IDH2-NC-OP- | /biotin/CGTCGGTCGTTCAAGCTGAAGAAGATGTGGAAAA | 5′ biotin |
| NO. 342 | 5 | GTCCCAATGGACCGACG | |
| SEQ ID | TP53-NC-OP- | /biotin/CGTCGGTCAGGGGGGAGCAGGGCTCACTCCAGGT | 5′ biotin |
| NO. 343 | 1 | GAGTGACCTGACCGACG | |
| SEQ ID | TP53-NC-OP- | /biotin/CGTCGGTCCCTTGGAACTCAAGGATGCCCAGGCTG | 5′ biotin |
| NO. 344 | 2 | GGAAGGAGGACCGACG | |
| SEQ ID | TP53-NC-OP- | /biotin/CGTCGGTCCGTGAGCGCTTCGAGATGTTCCGAGAG | 5′ biotin |
| NO. 345 | 3 | CTGAATGAGACCGACG | |
| SEQ ID | TP53-NC-OP- | /biotin/CGTCGGTCTACTTCTCCCCCTCCTCTGTTGCTGCAG | 5′ biotin |
| NO. 346 | 4 | ATCCGTGGACCGACG | |
| SEQ ID | TP53-NC-OP- | /biotin/CGTCGGTCGGGAGCCTCACCACGAGCTGCCCCCAG | 5′ biotin |
| NO. 347 | 5 | GGAGCACTGACCGACG | |
| SEQ ID | TP53-NC-OP- | /biotin/CGTCGGTCGACCGGCGCACAGAGGAAGAGAATCT | 5′ biotin |
| NO. 348 | 6 | CCGCAAGAAGACCGACG | |
| SEQ ID | TP53-NC-OP- | /biotin/CGTCGGTCCAGCTTTGAGGTGCGTGTTTGTGCCTG | 5′ biotin |
| NO. 349 | 7 | TCCTGGGAGACCGACG | |
| SEQ ID | TP53-NC-OP- | /biotin/CGTCGGTCTTCCTATCCTGAGTAGTGGTAATCTACT | 5′ biotin |
| NO. 350 | 8 | GGGACGGGACCGACG | |
| SEQ ID | TP53-NC-OP- | /biotin/CGTCGGTCCACCATCATCACACTGGAAGACTCCAG | 5′ biotin |
| NO. 351 | 9 | GTCAGGAGGACCGACG | |
| SEQ ID | TP53-NC-OP- | /biotin/CGTCGGTCGTTCCTGCATGGGCGGCATGAACCGGA | 5′ biotin |
| NO. 352 | 10 | GGCCCATCGACCGACG | |
| SEQ ID | TP53-NC-OP- | /biotin/CGTCGGTCGACTGTACCACCATCCACTACAACTAC | 5′ biotin |
| NO. 353 | 11 | ATGTGTAAGACCGACG | |
| SEQ ID | TP53-NC-OP- | /biotin/CGTCGGTCCTCATCTTGGGCCTGTGTTATCTCCTAG | 5′ biotin |
| NO. 354 | 12 | GTTGGCTGACCGACG | |
| SEQ ID | TP53-NC-OP- | /biotin/CGTCGGTCGTGTGGTGGTGCCCTATGAGCCGCCTG | 5′ biotin |
| NO. 355 | 13 | AGGTCTGGGACCGACG | |
| SEQ ID | TP53-NC-OP- | /biotin/CGTCGGTCGTGTGGAGTATTTGGATGACAGAAACA | 5′ biotin |
| NO. 356 | 14 | CTTTTCGACAGACCGACG | |
| SEQ ID | TP53-NC-OP- | /biotin/CGTCGGTCCCTCCTCAGCATCTTATCCGAGTGGAA | 5′ biotin |
| NO. 357 | 15 | GGAAATTTGACCGACG | |
| SEQ ID | TP53-NC-OP- | /biotin/CGTCGGTCGCCTCTGATTCCTCACTGATTGCTCTTA | 5′ biotin |
| NO. 358 | 16 | GGTCTGGGACCGACG | |
| SEQ ID | TP53-NC-OP- | /biotin/CGTCGGTCGCTCAGATAGCGATGGTGAGCAGCTGG | 5′ biotin |
| NO. 359 | 17 | GGCTGGAGGACCGACG | |
| SEQ ID | TP53-NC-OP- | /biotin/CGTCGGTCACGGAGGTTGTGAGGCGCTGCCCCCAC | 5′ biotin |
| NO. 360 | 18 | CATGAGCGGACCGACG | |
| SEQ ID | TP53-NC-OP- | /biotin/CGTCGGTCCCGCGCCATGGCCATCTACAAGCAGTC | 5′ biotin |
| NO. 361 | 19 | ACAGCACAGACCGACG | |
| SEQ ID | TP53-NC-OP- | /biotin/CGTCGGTCTGTGGGTTGATTCCACACCCCCGCCCG | 5′ biotin |
| NO. 362 | 20 | GCACCCGCGACCGACG | |
| SEQ ID | TP53-NC-OP- | /biotin/CGTCGGTCATGTTTTGCCAACTGGCCAAGACCTGC | 5′ biotin |
| NO. 363 | 21 | CCTGTGCAGACCGACG | |
| SEQ ID | TP53-NC-OP- | /biotin/CGTCGGTCCTTCCTCTTCCTACAGTACTCCCCTGCC | 5′ biotin |
| NO. 364 | 22 | CTCAACAGACCGACG | |
| SEQ ID | TP53-NC-OP- | /biotin/CGTCGGTCATCTGTTCACTTGTGCCCTGACTTTCAA | 5′ biotin |
| NO. 365 | 23 | CTCTGTCGACCGACG | |
| SEQ ID | TP53-NC-OP- | /biotin/CGTCGGTCGGGCAGCTACGGTTTCCGTCTGGGCTT | 5′ biotin |
| NO. 366 | 24 | CTTGCATTGACCGACG | |
| SEQ ID | TP53-NC-OP- | /biotin/CGTCGGTCCCCTGTCATCTTCTGTCCCTTCCCAGAA | 5′ biotin |
| NO. 367 | 25 | AACCTACGACCGACG | |
| SEQ ID | TP53-NC-OP- | /biotin/CGTCGGTCCCTACACCGGCGGCCCCTGCACCAGCC | 5′ biotin |
| NO. 368 | 26 | CCCTCCTGGACCGACG | |
| SEQ ID | TP53-NC-OP- | /biotin/CGTCGGTCAGAGGCTGCTCCCCCCGTGGCCCCTGC | 5′ biotin |
| NO. 369 | 27 | ACCAGCAGGACCGACG | |
| SEQ ID | TP53-NC-OP- | /biotin/CGTCGGTCCTGAAGACCCAGGTCCAGATGAAGCTC | 5′ biotin |
| NO. 370 | 28 | CCAGAATGGACCGACG | |
| SEQ ID | TP53-NC-OP- | /biotin/CGTCGGTCCCTATGGAAACTGTGAGTGGATCCATT | 5′ biotin |
| NO. 371 | 29 | GGAAGGGCGACCGACG | |
| SEQ ID | TP53-NC-OP- | /biotin/CGTCGGTCGCGTCGAGCCCCCTCTGAGTCAGGAAA | 5′ biotin |
| NO. 372 | 30 | CATTTTCAGACCGACG | |
| SEQ ID | TP53-NC-OP- | /biotin/CGTCGGTCCGGGTCACTGCCATGGAGGAGCCGCA | 5′ biotin |
| NO. 373 | 31 | GTCAGATCCGACCGACG | |
| SEQ ID | TP53-NC-OP- | /biotin/CGTCGGTCTCCCCACTTTTCCTCTTGCAGCAGCCAG | 5′ biotin |
| NO. 374 | 32 | ACTGCCTGACCGACG | |
| SEQ ID | IDH1-NC-OP- | /biotin/CGTCGGTCGCTTATGGGGATCAAGTAAGTCATGTT | 5′ biotin |
| NO. 375 | 1 | GGCAATAATGGACCGACG | |
| SEQ ID | IDH1-NC-OP- | /biotin/CGTCGGTCGAGTGGATGGGTAAAACCTATCATCAT | 5′ biotin |
| NO. 376 | 2 | AGGTCGTCGACCGACG | |
| SEQ ID | IDHI-NC-OP- | /biotin/CGTCGGTCGAGAAGCCATTATCTGCAAAAATATCC | 5′ biotin |
| NO. 377 | 3 | CCCGGCTTGACCGACG | |
| SEQ ID | IDH1-NC-OP- | /biotin/CGTCGGTCGGCACCATACGAAATATTCTGGGTGGC | 5′ biotin |
| NO. 378 | 4 | ACGGTCTTGACCGACG | |
| SEQ ID | IDH1-NC-OP- | /biotin/CGTCGGTCGTTGAGGAGTTCAAGTTGAAACAAATG | 5′ biotin |
| NO. 379 | 5 | TGGAAATCACCAAGACCGACG | |

After testing the basic effect of the NC probes of the present disclosure, Examples 4-8 further test the hybrid capture system based on the NC probes of the present disclosure and related parameters.

### Example 4: Optimal NC Probe Concentration Test

The difference in capture efficiency of NC probes with different concentrations for target genes is unknown, and through an experiment in which probes of different concentration gradients are set, the optimal probe concentration is sought. A specific experimental protocol is shown in Table 9 below, and a target region of 4.5 kb is designed according to the probe design concept of the present disclosure, and a Promega standard male (G1471 Promega-male) is used to fragment a sample to about 200-250 bp. For a specific experimental process, other variables are consistent except for different probe concentrations in experimental groups. Result data is shown in FIG. 7.

TABLE 9

| Experimental group | Probe concentration |
|---|---|
| Lib 1 | 2 fmol |
| Lib 2 | 2 fmol |
| Lib 3 | 4 fmol |
| Lib 4 | 4 fmol |
| Lib 5 | 6 fmol |
| Lib 6 | 6 fmol |
| Lib 7 | 10 fmol |
| Lib 8 | 10 fmol |

From result analysis of the Consensus depth, DS211 or SS information is directly proportional to the NC probe concentration. When the NC probe concentration is lower, less effective library information is captured, the higher the NC probe concentration, the richer the captured effective library information, but a too high NC probe concentration will lead to an excess of redundant NC probes in the system, resulting in a decrease in on-target rate. The optimal NC probe concentration used in this system is 6-10 fmol, with 6 fmol of the NC probe being more preferred.

### Example 5: Optimal Hybrid Capture Temperature Test

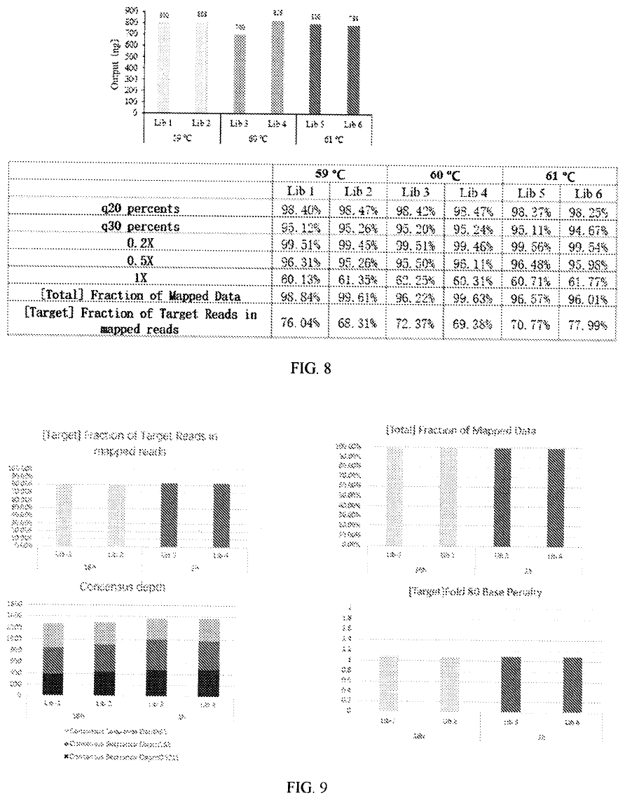

This system uses the NC probes, and the hybrid capture temperature needs to be selected according to the probe structure. In order to determine the optimal temperature conditions, a series of tests are performed. A specific experimental protocol is shown in Table 10 below. A target region of 4.5 kb is designed according to the design concept of the NC probes of the present disclosure. A Promega standard male is used to fragment a sample to about 200-250 bp. For a specific experimental process, other variables are consistent except for different hybrid capture temperatures in experimental groups. Result data is shown in FIG. 8.

TABLE 10

| Experimental group | Hybrid capture temperature |
|---|---|
| Lib 1 | 57° C. |
| Lib 2 | 60° C. |
| Lib 3 | 63° C. |

From result analysis of the library construction efficiency and the Consensus depth, the DS211 or SS content is affected by the hybrid capture temperature, the hybrid capture temperature of 60° C. performs better than the other two temperature conditions, and the capture efficiency and the on-target rate at the hybrid capture temperature of 60° C. are higher than those at the other hybrid capture temperatures.

To ensure that 60° C. is the optimal hybridization condition, and that this system is not too sensitive to the hybridization temperature, closer hybridization conditions are then tested to compare the difference in library capture efficiency under hybridization conditions of 59° C., 60° C., and 61° C. (see Table 11), other variables are consistent except for different hybrid capture temperatures in experimental groups, and the result data is shown in FIG. 8.

TABLE 11

| Experimental | Hybrid capture |
|---|---|
| group | temperature |
| Lib 1 | 59° C. |
| Lib 2 | 60° C. |
| Lib 3 | 61° C. |

From the above data analysis, hybridization temperatures from 59° C. to 61° C. show a superior capture efficiency, with 60°° C. being used as the final hybrid capture condition for this system.

### Example 6: Shortening Hybrid Capture Time

The hybridization time used in a traditional hybrid capture system is 16 hours, while the hybridization time used in the present disclosure can be reduced from 16 hours to 1 hour, and shortening the hybridization time does not affect the efficiency of the probes in capturing DNA samples.

An experiment is carried out by using the hybrid capture conditions of this system, a specific experimental protocol is shown in Table 12 below; a target region of 50 kb is first designed according to the design idea of the NC probes of the present disclosure, and a GW-OGTM800 standard is used to fragment a sample to about 200-250 bp.

The experimental process is as follows:
gDNA is fragmented to about 200 bp (Covaris ultrasonic fragmentation instrument), and end repair and adapter ligation are carried out, followed by purification of nucleic acid using an equal volume of Beads; and this specific purification process is as follows:
1. NadPrep® SP Beads are taken out in advance, vortexing is conducted for uniform mixing, and equilibration is conducted at room temperature for 30 minutes before use;
2. 80 μL of NadPrep® SP Beads is added to the adapter ligation product to be uniformly mixed, and the mixture is incubated at 25° C. for 5-10 minutes;
3. a PCR tube is instantaneously centrifuged, and placed on a magnetic rack for 5-10 minutes until liquid is completely clear, and a supernatant is discarded by pipetting with a pipette;
4. 200 μL of BW Buffer is added for washing once, the washed material is allowed to stand for 2 minutes, and a supernatant is discarded by pipetting; and
5. a hybridization reaction solution is added to the reaction system.

A hybridization system contains 6 fmol of probe, 1×Hyb Buffer, 1×Enhance, lug of Human Cot-1, and 100 pmmol of Blocker, and the configured hybridization reaction system is placed in a temperature controller for a reaction under the following conditions: denaturation at 95° C. for 2 minutes, and hybridization at 60° C. for 1 hour or 16 hours.

After completion of the hybridization reaction, the supernatant is transferred to a new PCR tube, and 10 μL of M270 Beads is added to the PCR reaction tube for hybrid capture at 60° C. for 20 minutes.

After the end of 20 minutes of capture, washing is separately performed once with an elution buffer I, an elution buffer II, and an elution buffer III.

After washing is completed, a PCR reaction system is added to the M270 Beads, wherein the PCR reaction system mainly includes 2×HiFi PCR Master Mix, 5 μL of Index Primer Mix, and 20 μL of TE; a PCR amplification procedure is started on a PCR temperature controller, and after the reaction is finished, the resulting product is purified by using 1×magnetic beads, and the purified product is sequenced on an Illumina® platform. Test result data is shown in FIG. 9.

TABLE 12

| Experimental | Library construction | Hybridization |
|---|---|---|
| group | and hybrid capture kit | time |
| Lib 1 | EASY Hybrid Capture System | 16 hours |
| Lib 2 | EASY Hybrid Capture System | 16 hours |
| Lib 3 | EASY Hybrid Capture System | 1 hour |
| Lib 4 | EASY Hybrid Capture System | 1 hour |

From result analysis of the Consensus depth, DS211 or SS information is directly proportional to the hybridization time, 90% or more of the efficient library have been captured after 1 hour of hybridization, with the final selection of the hybridization time of 1 hour, and the entire experimental process is controlled to be completed in one day.

### Example 7: Comparison of Capture of a Small Target Region by the NC Probes in a PCR-Free Mode With a Conventional Capture Process With Conventional Probes

In order to compare the performance of capture with NC probes in an optimized PCR-free mode with that of capture with traditional probes in a non-PCR-free mode for a small target region, an experiment is performed according to a grouping method in Table 13 below, wherein a group 1 uses a traditional manner to construct a targeted capture library, with the traditional hybrid capture system matched with probes of 120 nt; and a group 2 uses a system of the NC probes of the present disclosure to construct a PCR-free targeted capture library, capture probes are designed for a same region, the probes cover genomic exon regions, and the target region size is about 4 kb.

TABLE 13

| Experimental | | | |
|---|---|---|---|
| group | Library construction kit | Hybrid capture kit | |
| Group 1 | NadPrep DNA universal | NadPrep ® Hybrid | Control group |
| | library construction kit | Capture Reagents | |
| Group 2 | EASY Hybrid Capture | EASY Hybrid | Experimental |
| | System | Capture System | group |

Wherein a specific implementation process in the group 1 refers to a commercial instruction for a NadPrep® simple hybrid capture kit; while a specific experimental process in the group 2 refers to that in Example 6, and the hybridization time is fixed at 1 hour.

The data performance of this example is shown in Table 14. The mean coverage in the group 1 and the group 2 is close to 100%, while the on-target rate in the group 2 is 59%, which is higher than 11.73% in the group 1. It is obvious that the system of the NC probes of the present disclosure can effectively improve the on-target rate.

TABLE 14
Small target region capture efficiency higher than traditional hybrid capture

| | Group 1 | Group 2 |
|---|---|---|
| | Traditional | EASY Cap |
| Fraction of Target Reads in | 11.73% | 59% |
| mapped reads | | |
| Fraction of Mapped Reads | 99.29% | 99.32% |
| 0.2 × Mean coverage | 100.00% | 100% |
| 0.5 × Mean coverage | 98.92% | 100% |
| Fold 80 base penalty | 1.17 | 1.12 |

Note:
•[Target] Fraction of Target Reads in mapped reads: a proportion of target reads in mapped reads.
•Fraction of Mapped reads: a proportion of reads mapped to a human genome in all reads.
•0.2 × Mean Coverage: 0.2 × mean coverage percentage.
•0.5 × Mean coverage: 0.5 × mean coverage percentage.
•Fold 80 Base Penalty: sequencing multiples required to be increased to ensure that 80% of target bases reaches the original average coverage depth.

### Example 8: Detection Efficiency of Fusion Genes Higher Than Traditional Hybrid Capture

Fusion genes are produced when partial fragments of two genes are joined due to genome rearrangement. The fusion genes can be detected and analyzed by capturing and sequencing regions on both sides of a rearrangement breakpoint. Due to the fact that only part of rearrangement fragments across the breakpoint is the original sequence, for conventional probes, there may be a problem where only part of the fragments can be bound. The NC probes can also improve the detection ability of fusion genes through more probe binding possibilities.

An experiment is carried out according to a grouping method Table 15 below, wherein Group 1 uses a conventional manner to construct a targeted capture library, with a traditional hybrid capture system matched with probes of 120 nt, and probes covering the ROS1 intron 33 are designed to detect CD74-ROS1 fusion; and Group 2 uses the present disclosure to construct a targeted capture library, with capture probes designed for the same region, a target region of about 1 kb. Wherein a specific implementation process in the group 1 refers to the commercial instruction for the NadPrep® simple hybrid capture kit.

TABLE 15

| Experimental | | | |
|---|---|---|---|
| group | Library construction kit | Hybrid capture kit | |
| Group 1 | NadPrep DNA universal | NadPrep ® Hybrid | Control group |
| | library construction kit | Capture Reagents | |
| Group 2 | EASY Hybrid Capture | EASY Hybrid | Experimental |
| | System | Capture System | group |

The sample is a pan-tumor 800 gDNA standard (GW-OGTM800) containing multiple digital PCR verified mutation sites, one of which is CD74-ROS1 Fusion, and this site has a theoretical mutation frequency of 6%.

The specific experimental process in the group 2 refers to that in Example 6, and result data is shown in Table 16 below.

TABLE 16
Detection efficiency of fusion genes higher than traditional hybrid capture

| | Group 1 | Group 2 |
|---|---|---|
| | Traditional | EASY Cap |
| Fraction of Target Reads in mapped | 10.5% | 52.3% |
| reads | | |
| Fraction of Mapped Reads | 98.08% | 99.88% |
| 0.2 × Mean coverage | 98.46% | 100.00% |
| 0.5 × Mean coverage | 89.57% | 88.24% |
| CD74-ROS1 (a theoretical value of | 1.1% | 5.8% |
| 6%) | | |

Note:
•[Target] Fraction of Target Reads in mapped reads: a proportion of target reads in mapped reads.
•Fraction of Mapped reads: a proportion of reads mapped to a human genome in all reads.
•0.2 × Mean Coverage: 0.2 × mean coverage percentage.
•0.5 × Mean coverage: 0.5 × mean coverage percentage.
•Fold 80 Base Penalty: sequencing multiples required to be increased to ensure that 80% of target bases reaches the original average coverage depth.

Fusion sites are often located within a repeating region, and a probe design within the repeating region is something of a capture challenge. However, the use of the NC probes in this system shows certain advantages for the detection of the fusion genes. The GW-OGTM800 standard in this experiment contains a set of CD74-ROS1 fusion genes with a mutation frequency of 5% as verified by digital PCR; and the Group 1 and the Group 2 use probes covering the same region for hybrid capture, and the frequency of detecting fusion genes by the traditional method is about 1.1%, while the frequency of detecting fusion genes by the optimized system of the present disclosure is 5.8%.

The above are only preferred Examples of the present disclosure, and are not used to limit the present disclosure. All documents mentioned in the present disclosure are hereby incorporated by reference in their entirety. Further, it should be understood that after reading the above teachings of the present disclosure, those skilled in the art can make various changes or modifications to the present disclosure within the spirit and principles of the present disclosure, and these equivalent modifications also fall within the scope defined in the claims of the present application.

1. A liquid-phase hybrid capture method, comprising the following steps of:
(1) probe design: designing a pool of probes based on nucleic acid target regions, wherein each probe comprises a probe binding sequence complementarily pairing with another probe, and a target specific sequence complementarily pairing with a nucleic acid target sequence, synthesizing each oligonucleotide based on corresponding sequence, and modifying a 5′ or 3′ end with a biomarker;
(2) library construction;
(3) hybrid capture
i. configuring a hybridization system: wherein the hybridization system comprises the probes; and
ii. carrying out a hybridization reaction: placing the hybridization system at 57-63° C. for hybridization for 1-2 h;
(4) product capture: after the hybridization reaction is completed, adding streptavidin magnetic beads to the reaction system for hybrid capture;
(5) product elution: after capture is completed, separately washing the captured product once with an elution buffer I, an elution buffer II and an elution buffer III; and
(6) product amplification and purification: after the washing is completed, adding a PCR reaction system for a PCR amplification procedure, and after the reaction is completed, performing purification by using magnetic beads.

2. The method according to claim 1, wherein the probe binding sequence comprises a first probe binding sequence and a second probe binding sequence.

3. The method according to claim 2, wherein a 5′ end of each probe has a first probe binding sequence complementarily pairing with a 3′ end of another probe, and a 3′ end of each probe has a second probe binding sequence complementarily pairing with a 5′ end of another probe.

4. The method according to claim 1, wherein the nucleic acid is from fresh tissue, frozen tissue, paraffin embedded tissue, hydrothorax and ascites, plasma or exfoliated tumor cell tissue.

5. The method according to claim 1, wherein the nucleic acid is plasma free DNA, genomic DNA or RNA.

6. The method according to claim 1, wherein the library construction is to construct a DNA library based on nucleic acid fragment size of 200-250 bp.

7. The method according to claim 1, wherein the hybridization system comprises 2-10 fmol of the probe, 1×Hyb Buffer, 1×Enhance, lug of Human Cot-1, and 100 pmmol of Blocker.

8. The method according to claim 7, wherein the hybridization system comprises 6 fmol of the probe.

9. (canceled)

10. (canceled)

11. The method according to claim 1, wherein the PCR reaction system comprises 2×HiFi PCR Master Mix, 5 μL of Index Primer Mix and 20 μL of TE.

12. The method according to claim 1, wherein the library construction comprises end repair and adapter ligation of nucleic acid fragments.

13. The method according to claim 1, wherein formula of the elution buffer I is 5×SSPE, and 0.5-5% of SDS; formula of the elution buffer II is 2×SSPE, and 0.05-0.5% of SDS; and formula of the elution buffer III is 0.1×SSPE, and 0.005%-0.05% of SDS.

14. A liquid-phase hybrid capture kit, comprising the following components: probes, a hybridization reaction solution, an elution buffer, and nucleic acid purification magnetic beads; wherein each probe comprises a probe binding sequence complementarily pairing with another probe, and a target specific sequence complementarily pairing with a nucleic acid target sequence.

15. The kit according to claim 14, further comprising an end repair enzyme mixture, an end repair reaction buffer, a molecular tag-containing adapter, library amplification primers, a PCR premix, an adapter blocker, a DNA blocker, a hybridization enhancer, a magnetic bead wash buffer, and capture library PCR primers.

16. The method of claim 1 used in genomic target region capture.

17. The method according to claim 16, wherein the target region capture is used for low-frequency mutation detection, chromosome copy number variation analysis, and insertion/deletion, microsatellite instability or fusion gene detection in nucleic acid fragments; or is used for targeted metagenomic next-generation sequencing (mNGS), and epidemiological detection of pathogens.
